(19) United States
(12) Patent Application Publication
(10) Pub. No.: US 2026/0083663 A1
(43) Pub. Date: Mar. 26, 2026

(54) BOTANICAL COMPOSITIONS AND METHODS OF PRODUCTION AND USE THEREOF

(71) Applicant: ARKANSAS STATE UNIVERSITY-JONESBORO, State University, AR (US)

(72) Inventors: Luis Fabricio MEDINA-BOLIVAR, Memphis, TN (US); Lingling FANG, Jonesboro, AR (US); Amit Raj SHARMA, Jonesboro, AR (US)

(21) Appl. No.: 19/109,294

(22) PCT Filed: Sep. 11, 2023
(86) PCT No.: PCT/US2023/032434

§ 371 Date: Mar. 6, 2025

Related U.S. Application Data
(60) Provisional application No. 63/405,268, filed on Sep. 9, 2022.

(51) Int. Cl. A61K 8/9789; A01H 4/00; A61Q 19/02

(52) U.S. Cl. CPC A61K 8/9789; A01H 4/002; A01H 4/005; A61Q 19/02; A61K 2800/805

(57) ABSTRACT

This invention is directed to botanical compositions comprising plant extracts and methods of preparing the same.

This application claims priority from U.S. Provisional Application No. 63/405,268, filed on Sep. 9, 2022, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to botanical compositions comprising plant extracts, methods of preparing the same, and methods of using the same.

### BACKGROUND OF THE INVENTION

Plant products can have applications as dietary supplements, cosmeceuticals, pharmaceuticals, and agrochemicals. Due to the complexity of their chemical structures, the production of these products is not feasible via organic synthesis and therefore their availability depends on extraction from plant sources. Methods of plant extraction are limited.

### SUMMARY OF THE INVENTION

Aspects of the invention are drawn to methods for producing a botanical composition. In embodiments, the method comprises obtaining plant tissue from Morus alba; producing a root culture from the plant tissue; and stimulating production of a botanical composition from said root culture using an elicitation medium, wherein the elicitation medium comprises a combination of elicitors.

In embodiments, the method further comprises culturing the plant tissue for a period of time in a culture medium, such that the plant tissue produces a root culture. For example, the time in the culture medium can comprise less than 1 day, about 1 day, about 5 days, about 10 days, about 20 days, about 25 days, about 30 days, or more than 30 days.

In embodiments, the method further comprises incubating the root culture in the elicitation medium for about 4 hours, about 12 hours, about 24 hours, about 48 hours, about 96 hours, or about 192 hours.

In embodiments, the root culture comprises root tissue, medium of said root culture, or a combination thereof.

In embodiments, the method further comprises the step of isolating the botanical composition from the root culture, thereby providing a botanical extract.

In embodiments, the method comprises contacting the root culture with a non-polar solvent and extracting the organic layer from the aqueous layer. For example, the non-polar solvent comprises ethyl acetate.

In embodiments, the method comprises contacting the root culture with a polar solvent and extracting the organic layer from the aqueous layer. For example, the polar solvent comprises water, an alcohol, a glycol, or a combination thereof. For example, the alcohol comprises methanol, ethanol, propanol, butanol, pentanol, and hexanol. For example, the glycol comprises ethylene glycol or propylene glycol.

In embodiments, the elicitors are selected from the group consisting of cyclodextrin, magnesium chloride, hydrogen peroxide, or a combination thereof.

In embodiments, the elicitors can be present in a concentration comprising about 18 g/L cyclodextrin, about 1 mM magnesium chloride, about 3 mM hydrogen peroxide, or any combination thereof.

In embodiments, the botanical composition comprises about 0.01 mg/g to about 50 mg/g of an aryl benzofuran compound or derivative thereof per dry weight of root tissue. For example, the aryl benzofuran compound is a compound comprising Formula (I):

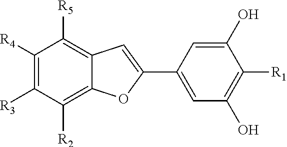

wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof. For example, the compound comprises Formula (I), wherein Formula I comprises:

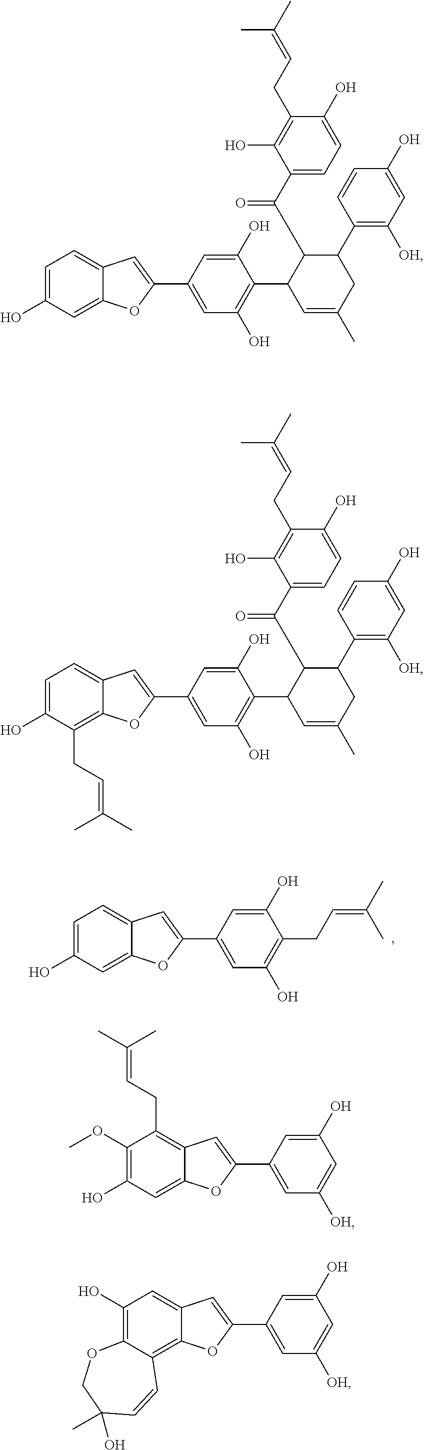

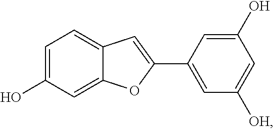 [SMILES: Oc1cc(O)cc(-c2cc3ccc(O)cc3o2)c1]

or a glucosidyl derivative thereof.

In embodiments, the botanical composition is characterized by a spectrum that is substantially the same as the spectrum shown in FIGS. 19, 21, 26, 28, 30, and/or 43.

In embodiments, the botanical composition further comprises a stilbenoid, flavone, or derivative thereof. For example, the stilbenoid or derivative thereof comprises resveratrol, oxyresveratrol, mulberroside, prenyl-resveratrol, prenyloxyresveratrol, or combination thereof.

In embodiments, oxyresveratrol comprises about 0.1 mg/g to about 10.0 mg/g per dry weight of root tissue.

In embodiments, resveratrol comprises about 0.1 mg/g to about 10.0 mg/g per dry weight of root tissue.

In embodiments, prenyl-resveratrol comprises about 0.1 mg/g to about 5.0 mg/g per dry weight of root tissue.

In embodiments, mulberroside A comprises about 0.1 mg/g to about 20.0 mg/g per dry weight of root tissue.

In embodiments, prenyloxyresveratrol comprises about 0.1 mg/g to about 20 mg/g per dry weight of root tissue.

In embodiments, the flavone or derivative thereof comprises kuwanon or a derivative thereof. For example, the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E. For example, kuwanon C comprises about 0.1 mg/g to about 10.0 mg/g per dry weight of root tissue. For example, kuwanon H comprises about 0.1 mg/g to about 10.0 mg/g per dry weight of root tissue. For example, the kuwanon G analog comprises about 0.1 mg/g to about 10.0 mg/g per dry weight of root tissue.

In embodiments, the plant tissue comprises the root and/or the aerial parts of Morus alba.

In embodiments, the root culture is a not a hairy root culture.

In embodiments, the root culture is a hairy root culture.

Aspects of the invention are also drawn towards a method of producing moracin in a botanical composition, the method comprising obtaining plant tissue from Morus alba; producing a root culture from the plant tissue; and stimulating production of a botanical composition from said root culture using a combination of elicitors.

In embodiments, the method further comprises culturing the plant tissue for a period of time in a culture medium, such that the plant tissue produces a root culture. For example, the time in the culture medium can comprise less than 1 day, about 1 day, about 5 days, about 10 days, about 20 days, about 25 days, about 30 days, or more than 30 days.

In embodiments, the method further comprises incubating the root culture in the elicitation medium for about 4 hours, about 12 hours, about 24 hours, about 48 hours, about 96 hours, or about 192 hours.

In embodiments, the root culture comprises root tissue, medium of said root culture, or a combination thereof.

In embodiments, the method further comprises the step of isolating the botanical composition from the root culture, thereby providing a botanical extract.

In embodiments, the method comprises contacting the root culture with a non-polar solvent and extracting the organic layer from the aqueous layer. For example, the non-polar solvent comprises ethyl acetate.

In embodiments, the method comprises contacting the root culture with a polar solvent and extracting the organic layer from the aqueous layer. For example, the polar solvent comprises water, an alcohol, a glycol, or a combination thereof. For example, the alcohol comprises methanol, ethanol, propanol, butanol, pentanol, and hexanol. For example, the glycol comprises ethylene glycol or propylene glycol.

In embodiments, the elicitors are selected from the group consisting of cyclodextrin, magnesium chloride, hydrogen peroxide, or a combination thereof.

In embodiments, the elicitors can be present in a concentration comprising about 18 g/L cyclodextrin, about 1 mM magnesium chloride, about 3 mM hydrogen peroxide, or any combination thereof.

In embodiments, the botanical composition comprises about 0.01 mg/g to about 50 mg/g of an aryl benzofuran compound or derivative thereof per dry weight of root tissue. For example, the aryl benzofuran compound is a compound comprising Formula (I):

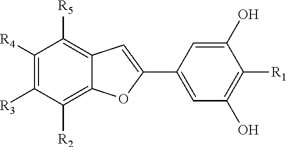

wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof. For example, the compound comprises Formula (I), wherein Formula I comprises:

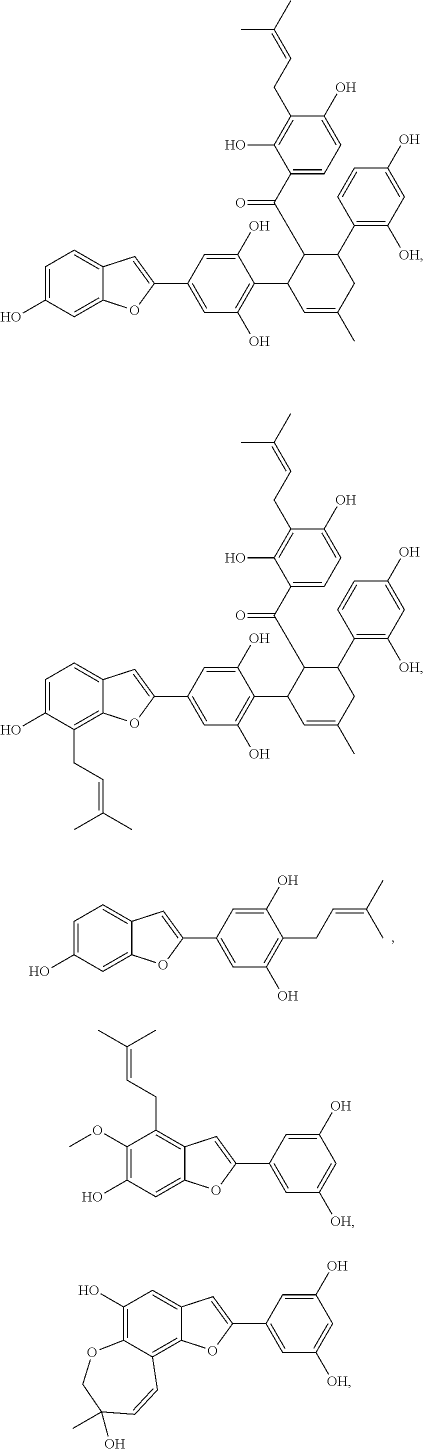

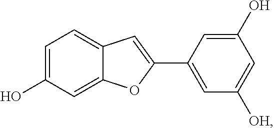 [SMILES: Oc1cc(O)cc(-c2cc3ccc(O)cc3o2)c1]

or a glucosidyl derivative thereof.

In embodiments, the botanical composition is characterized by a spectrum that is substantially the same as the spectrum shown in FIGS. 19, 21, 26, 28, 30, and/or 43.

In embodiments, the botanical composition further comprises a stilbenoid, flavone, or derivative thereof. For example, the stilbenoid or derivative thereof comprises resveratrol, oxyresveratrol, mulberroside, prenyl-resveratrol, prenyloxyresveratrol, or combination thereof.

In embodiments, oxyresveratrol comprises about 0.1 mg/g to about 10.0 mg/g per dry weight of root tissue.

In embodiments, resveratrol comprises about 0.1 mg/g to about 10.0 mg/g per dry weight of root tissue.

In embodiments, prenyl-resveratrol comprises about 0.1 mg/g to about 5.0 mg/g per dry weight of root tissue.

In embodiments, mulberroside A comprises about 0.1 mg/g to about 20.0 mg/g per dry weight of root tissue.

In embodiments, prenyloxyresveratrol comprises about 0.1 mg/g to about 20 mg/g per dry weight of root tissue.

In embodiments, the flavone or derivative thereof comprises kuwanon or a derivative thereof. For example, the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E. For example, kuwanon C comprises about 0.1 mg/g to about 10.0 mg/g per dry weight of root tissue. For example, kuwanon H comprises about 0.1 mg/g to about 10.0 mg/g per dry weight of root tissue. For example, the kuwanon G analog comprises about 0.1 mg/g to about 10.0 mg/g per dry weight of root tissue.

In embodiments, the plant tissue comprises the root and/or the aerial parts of Morus alba.

In embodiments, the root culture is a not a hairy root culture.

In embodiments, the root culture is a hairy root culture.

Aspects of the invention are also drawn to a botanical composition produced by a method as described herein.

In embodiments, the botanical composition comprises about 0.01 mg/g to about 50 mg/g of an aryl benzofuran compound or derivative thereof per dry weight of root tissue.

In embodiments, the aryl benzofuran compound is a compound comprising Formula (I):

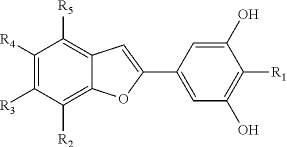

wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof. For example, the compound comprising Formula (I) comprises:

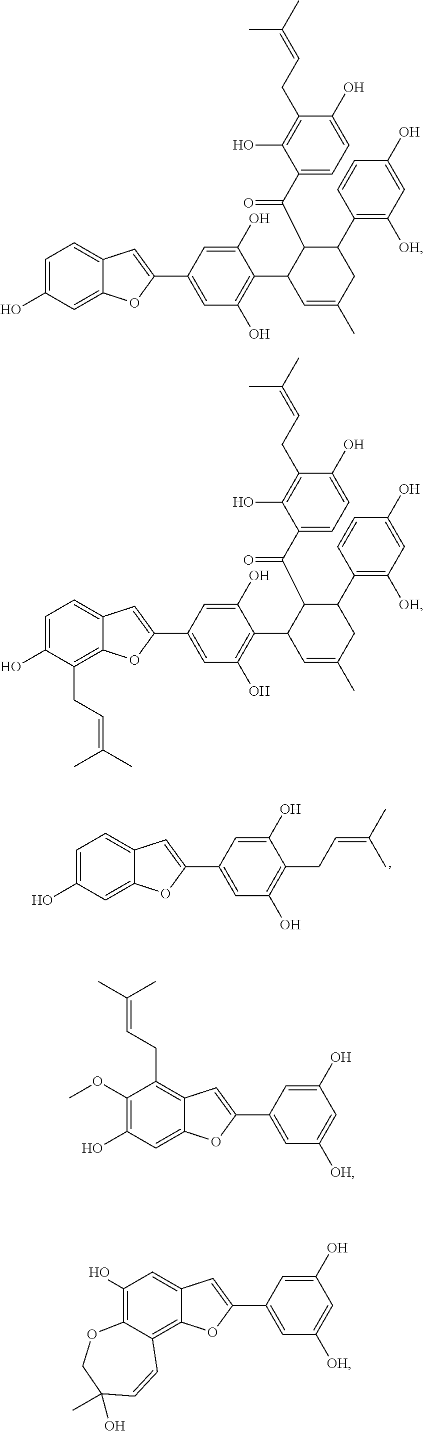

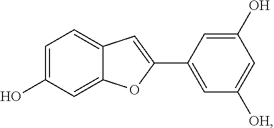 [SMILES: Oc1cc(O)cc(-c2cc3ccc(O)cc3o2)c1]

or a glucosidyl derivative thereof.

In embodiments, the botanical composition is characterized by a spectrum that is substantially the same as the spectrum shown in FIGS. 19, 21, 26, 28, 30 and/or 43.

In embodiments, the botanical composition further comprises a stilbenoid, flavone, or derivative thereof. For example, the stilbenoid or derivative thereof comprises resveratrol, oxyresveratrol, mulberroside, prenyl-resveratrol, prenyloxyresveratrol, or combination thereof.

In embodiments, oxyresveratrol comprises about 0.1 mg/g to about 10.0 mg/g per dry weight of root tissue.

In embodiments, resveratrol comprises about 0.1 mg/g to about 10.0 mg/g per dry weight of root tissue.

In embodiments, prenyl-resveratrol comprises about 0.1 mg/g to about 5.0 mg/g per dry weight of root tissue.

In embodiments, mulberroside A comprises about 0.1 mg/g to about 20.0 mg/g per dry weight of root tissue.

In embodiments, prenyloxyresveratrol comprises about 0.1 mg/g to about 20 mg/g per dry weight of root tissue.

In embodiments, the flavone or derivative thereof comprises kuwanon or a derivative thereof. For example, the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E.

In embodiments, kuwanon C comprises about 0.1 mg/g to about 10.0 mg/g per dry weight of root tissue.

In embodiments, kuwanon H comprises about 0.1 mg/g to about 10.0 mg/g per dry weight of root tissue.

In embodiments, the kuwanon G analog comprises about 0.1 mg/g to about 10.0 mg/g per dry weight of root tissue.

In embodiments, the botanical composition further comprises a carrier, excipient or dilutant.

Still further, aspects of the invention are drawn towards a fraction or an isolated component of a botanical composition produced by a method as described herein. In embodiments, the fraction or isolated component can further comprise a carrier, excipient, or diluent.

Aspects of the invention are also drawn towards a method for producing a cosmetic composition. In embodiments, the method comprises obtaining plant tissue from Morus alba; producing a root culture from the plant tissue; stimulating production of a botanical composition from said root culture using a combination of elicitors; isolating the botanical composition from the root culture, thereby providing a botanical extract; and generating a cosmetic composition comprising the botanical extract.

In embodiments, the method further comprises culturing the plant tissue for a period of time in a culture medium, such that the plant tissue produces a root culture. For example, the time in the culture medium can comprise less than 1 day, about 1 day, about 5 days, about 10 days, about 20 days, about 25 days, about 30 days, or more than 30 days.

In embodiments, the method further comprises incubating the root culture in the elicitation medium for about 4 hours, about 12 hours, about 24 hours, about 48 hours, about 96 hours, or about 192 hours.

In embodiments, the root culture comprises root tissue, medium of said root culture, or a combination thereof.

In embodiments, the method further comprises the step of isolating the botanical composition from the root culture, thereby providing a botanical extract.

In embodiments, the method comprises contacting the root culture with a non-polar solvent and extracting the organic layer from the aqueous layer. For example, the non-polar solvent comprises ethyl acetate.

In embodiments, the method comprises contacting the root culture with a polar solvent and extracting the organic layer from the aqueous layer. For example, the polar solvent comprises water, an alcohol, a glycol, or a combination thereof. For example, the alcohol comprises methanol, ethanol, propanol, butanol, pentanol, and hexanol. For example, the glycol comprises ethylene glycol or propylene glycol.

In embodiments, the elicitors are selected from the group consisting of cyclodextrin, magnesium chloride, hydrogen peroxide, or a combination thereof.

In embodiments, the elicitors can be present in a concentration comprising about 18 g/L cyclodextrin, about 1 mM magnesium chloride, about 3 mM hydrogen peroxide, or any combination thereof.

In embodiments, the botanical composition comprises about 0.01 mg/g to about 50 mg/g of an aryl benzofuran compound or derivative thereof per dry weight of root tissue. For example, the aryl benzofuran compound is a compound comprising Formula (I):

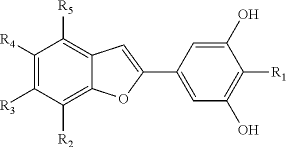

wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof. For example, the compound comprises Formula (I), wherein Formula I comprises:

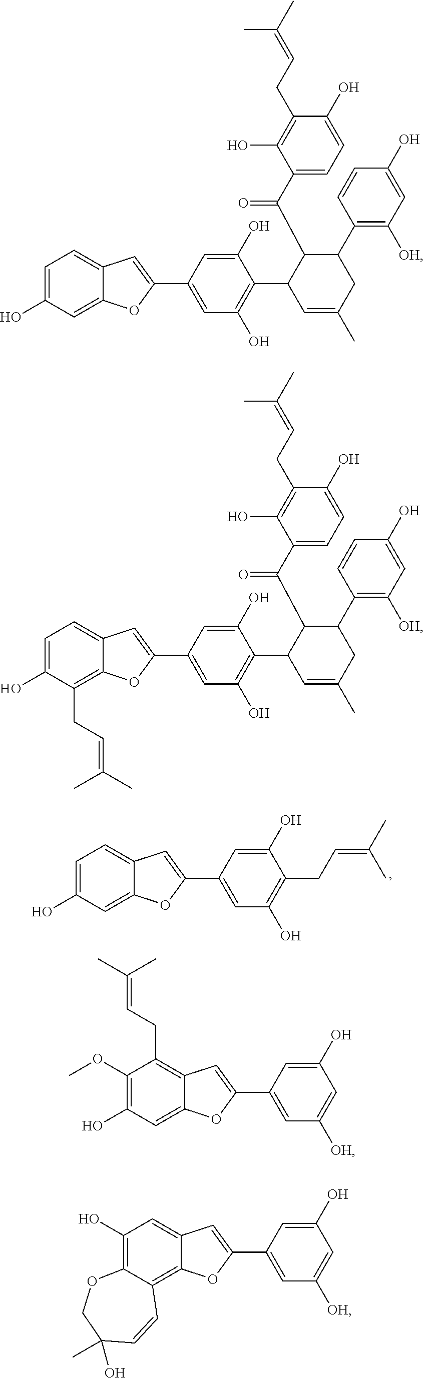

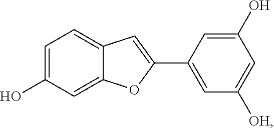 [SMILES: Oc1cc(O)cc(-c2cc3ccc(O)cc3o2)c1]

or a glucosidyl derivative thereof.

In embodiments, the botanical composition is characterized by a spectrum that is substantially the same as the spectrum shown in FIGS. 19, 21, 26, 28, 30, and/or 43.

In embodiments, the botanical composition further comprises a stilbenoid, flavone, or derivative thereof. For example, the stilbenoid or derivative thereof comprises resveratrol, oxyresveratrol, mulberroside, prenyl-resveratrol, prenyloxyresveratrol, or combination thereof.

In embodiments, oxyresveratrol comprises about 0.1 mg/g to about 10.0 mg/g per dry weight of root tissue.

In embodiments, resveratrol comprises about 0.1 mg/g to about 10.0 mg/g per dry weight of root tissue.

In embodiments, prenyl-resveratrol comprises about 0.1 mg/g to about 5.0 mg/g per dry weight of root tissue.

In embodiments, mulberroside A comprises about 0.1 mg/g to about 20.0 mg/g per dry weight of root tissue.

In embodiments, prenyloxyresveratrol comprises about 0.1 mg/g to about 20 mg/g per dry weight of root tissue.

In embodiments, the flavone or derivative thereof comprises kuwanon or a derivative thereof. For example, the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E. For example, kuwanon C comprises about 0.1 mg/g to about 10.0 mg/g per dry weight of root tissue. For example, kuwanon H comprises about 0.1 mg/g to about 10.0 mg/g per dry weight of root tissue. For example, the kuwanon G analog comprises about 0.1 mg/g to about 10.0 mg/g per dry weight of root tissue.

In embodiments, the plant tissue comprises the root and/or the aerial parts of Morus alba.

In embodiments, the root culture is a not a hairy root culture.

In embodiments, the root culture is a hairy root culture.

Aspects of the invention are also drawn towards a method of producing moracin in a botanical composition, the method comprising obtaining plant tissue from Morus alba; producing a root culture from the plant tissue; and stimulating production of a botanical composition from said root culture using a combination of elicitors.

In embodiments, the method further comprises culturing the plant tissue for a period of time in a culture medium, such that the plant tissue produces a root culture. For example, the time in the culture medium can comprise less than 1 day, about 1 day, about 5 days, about 10 days, about 20 days, about 25 days, about 30 days, or more than 30 days.

In embodiments, the method further comprises incubating the root culture in the elicitation medium for about 4 hours, about 12 hours, about 24 hours, about 48 hours, about 96 hours, or about 192 hours.

In embodiments, the root culture comprises root tissue, medium of said root culture, or a combination thereof.

In embodiments, the method further comprises the step of isolating the botanical composition from the root culture, thereby providing a botanical extract.

In embodiments, the method comprises contacting the root culture with a non-polar solvent and extracting the organic layer from the aqueous layer. For example, the non-polar solvent comprises ethyl acetate.

In embodiments, the method comprises contacting the root culture with a polar solvent and extracting the organic layer from the aqueous layer. For example, the polar solvent comprises water, an alcohol, a glycol, or a combination thereof. For example, the alcohol comprises methanol, ethanol, propanol, butanol, pentanol, and hexanol. For example, the glycol comprises ethylene glycol or propylene glycol.

In embodiments, the elicitors are selected from the group consisting of cyclodextrin, magnesium chloride, hydrogen peroxide, or a combination thereof.

In embodiments, the elicitors can be present in a concentration comprising about 18 g/L cyclodextrin, about 1 mM magnesium chloride, about 3 mM hydrogen peroxide, or any combination thereof.

In embodiments, the botanical composition comprises about 0.01 mg/g to about 50 mg/g of an aryl benzofuran compound or derivative thereof per dry weight of root tissue. For example, the aryl benzofuran compound is a compound comprising Formula (I):

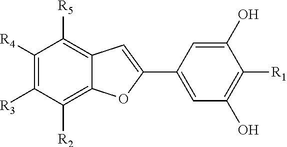

wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof. For example, the compound comprises Formula (I), wherein Formula I comprises:

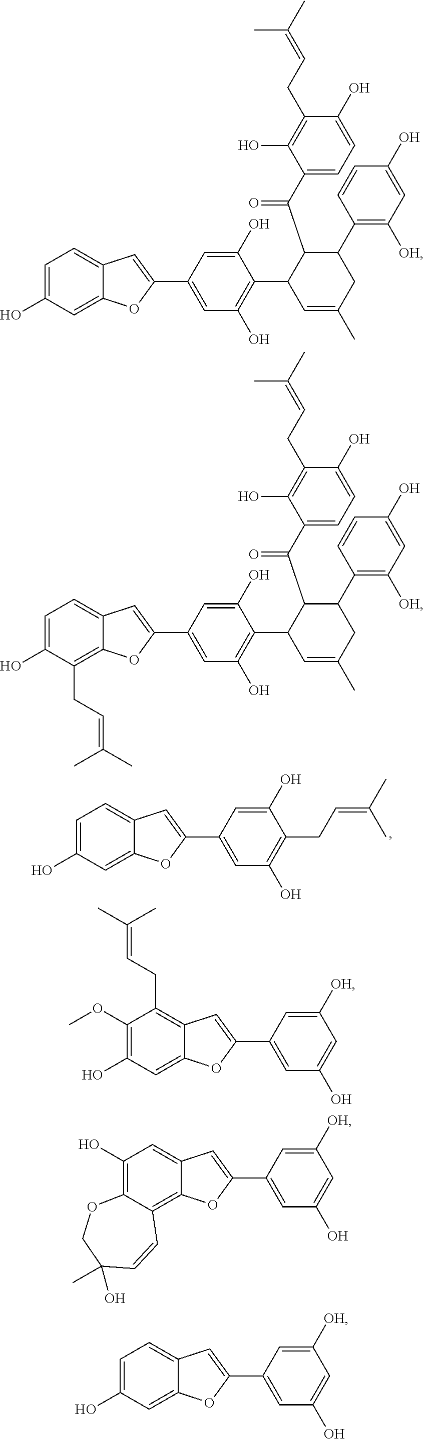

or a glucosidyl derivative thereof.

In embodiments, the botanical composition is characterized by a spectrum that is substantially the same as the spectrum shown in FIGS. 19, 21, 26, 28, 30, and/or 43.

In embodiments, the botanical composition further comprises a stilbenoid, flavone, or derivative thereof. For example, the stilbenoid or derivative thereof comprises resveratrol, oxyresveratrol, mulberroside, prenyl-resveratrol, prenyloxyresveratrol, or combination thereof.

In embodiments, oxyresveratrol comprises about 0.1 mg/g to about 10.0 mg/g per dry weight of root tissue.

In embodiments, resveratrol comprises about 0.1 mg/g to about 10.0 mg/g per dry weight of root tissue.

In embodiments, prenyl-resveratrol comprises about 0.1 mg/g to about 5.0 mg/g per dry weight of root tissue.

In embodiments, mulberroside A comprises about 0.1 mg/g to about 20.0 mg/g per dry weight of root tissue.

In embodiments, prenyloxyresveratrol comprises about 0.1 mg/g to about 20 mg/g per dry weight of root tissue.

In embodiments, the flavone or derivative thereof comprises kuwanon or a derivative thereof. For example, the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E. For example, kuwanon C comprises about 0.1 mg/g to about 10.0 mg/g per dry weight of root tissue. For example, kuwanon H comprises about 0.1 mg/g to about 10.0 mg/g per dry weight of root tissue. For example, the kuwanon G analog comprises about 0.1 mg/g to about 10.0 mg/g per dry weight of root tissue.

In embodiments, the plant tissue comprises the root and/or the aerial parts of Morus alba.

In embodiments, the root culture is a not a hairy root culture.

In embodiments, the root culture is a hairy root culture.

Still further, aspects of the invention are drawn to a cosmetic composition or a pharmaceutical composition produced by a method as described herein.

In embodiments, the cosmetic composition or pharmaceutical composition further comprising a component selected from a sulfite, a lemon extract, a saxifrage extract, a grapefruit extract, a grape extract, an oughon extract, and combinations thereof.

In embodiments, the cosmetic composition or pharmaceutical composition further comprises a carrier, excipient, or diluent.

In embodiments, the cosmetic composition or pharmaceutical composition is a lotion, a gel, an emulsion, a balm, a stick, an oil, a milk, a cream, an ointment, or a dusting powder.

In embodiments, the cosmetic composition or pharmaceutical composition comprises one or more additives selected from the group consisting of solvents, thickeners, softeners, moisturizers, sealers, emollients, wetting agents, chelating agents, preservatives, anti-foaming agents, perfumes and emulsifiers.

Aspects of the invention are also drawn towards a method of decreasing the melanin content of human skin or hair. For example, the method comprises applying to skin or hair an effective amount of the cosmetic composition or pharmaceutical composition as described herein.

Still further, aspects of the invention are drawn towards a method of modulating skin pigmentation or skin color in a subject. For example, the method comprises applying to skin of the subject an effective amount of the cosmetic composition or pharmaceutical composition as described herein.

In embodiments, modulating skin pigmentation or skin color comprises treating a skin pigmentation disorder, wherein the skin pigmentation disorder comprises age spots, sun damage, or hyperpigmentation.

In embodiments, modulating skin pigmentation or skin color comprises whitening, lightening, brightening, or increasing radiance in at least a portion of skin of a subject.

In embodiments, the method comprises applying the cosmetic composition to the skin or hair of a subject.

Aspects of the invention are also drawn towards a method for inhibiting melanogenesis, for decreasing pigmentation of skin, for decreasing pigmentation of skin appendages, or for decreasing pigment spots on skin. For example, the method comprises topically administering an effective amount of the botanical composition as described herein, or a composition comprising said botanical composition, or the cosmetic composition or pharmaceutical composition as described herein, to an animal in need thereof.

In embodiments, the botanical composition, the composition comprising said botanical composition, or the cosmetic composition or pharmaceutical composition is administered topically onto skin or skin appendages of the animal.

In embodiments, the botanical composition, the composition comprising said botanical composition, or the cosmetic composition or pharmaceutical composition is applied topically onto all or part of the animal body.

In embodiments, the animal is a human. For example, the botanical composition, the composition comprising said botanical composition, or the cosmetic composition or pharmaceutical composition is applied topically onto at least one part of a human body selected from a hand, neck, neckline, stomach, arm, thigh, hip, waist, face, and combinations thereof.

Aspects of the invention are drawn to a method for treating a pathological condition including pigment dysregulation. In embodiments, the method comprises administering an effective amount of the botanical composition as described herein, or a composition comprising said botanical composition, or the cosmetic composition or pharmaceutical composition as described herein to an animal in need thereof.

In embodiments, the subject is a human subject.

In embodiments, the pathological condition is a pathological hyperpigmentation.

In embodiments, the pathological condition is selected from Addison's disease, liver failure, purpura, melanoma, or dermatosis papulose nigra.

In embodiments, the botanical composition, the composition comprising said botanical composition, or the cosmetic composition or pharmaceutical composition is administered topically.

In embodiments, the cosmetic composition as described herein comprises botanical extract present in a concentration between 1×10−4% and 50% (w/w), between 1×10−3% and 20% (w/w), between 0.01% and 10% (w/w), or between 0.01% and 5% (w/w), relative to the total weight of the composition.

### BRIEF DESCRIPTION OF THE FIGURES

FIG. 1 shows (panel A) a Morus alba (Spain) 1-month-old seedling and (panel B) Morus alba (Spain) sample in a Petri dish.

FIG. 2 shows leaves wounded by Agrobacterium rhizogenes ATCC 15834 at Day 12 (panel A) and Day 21 (panel B).

FIG. 3 shows Morus alba (Spain) after day 24 of wounding.

FIG. 4 shows non-limiting examples of germination of seeds of Morus alba.

FIG. 5 shows non-limiting examples of root cultures grown in different cultures.

FIG. 6 shows non-limiting examples of media samples before, at 48 hours, 96 hours, 144 hrs, and 192 hours.

FIG. 7 shows chromatograms of medium of normal root at 265 nm.

FIG. 8 shows chromatograms of medium of normal root at 320 nm.

FIG. 9 shows HPLC chromatograms at 265 nm and 320 nm of medium extracts of control and elicited normal root cultures of Ukraine white mulberry.

FIG. 10 shows HPLC chromatograms at 320 nm of medium extracts of control and elicited normal root cultures of white mulberry. The UV spectra of the compounds detected in the elicited normal roots are shown. Each UV spectrum shows the HPLC retention time of the detected compound.

FIG. 11 shows graphs of compounds found in medium of normal root (room temperature) in mg/L (panel A) and mg/g DW (panel B).

FIG. 12 shows UV chromatograms of normal roots at 265 nm.

FIG. 13 shows UV chromatograms of normal roots at 320 nm.

FIG. 14 shows UV chromatograms of normal control, elicited, and normal root medium at 192 hrs at 320 nm.

FIG. 15 shows HPLC chromatograms at 320 nm of control and elicited root tissue extracts from normal roots of white mulberry. The UV spectra of the compounds detected in the elicited normal roots are shown. Each UV spectrum shows the HPLC retention time of the detected compound.

FIG. 16 shows graphs of compounds found in medium of normal root cultures at room temperatures in mg/g dry weight of the root.

FIG. 17 shows non-limiting, examples of yields of compounds found in the medium and the root of normal root extraction.

FIG. 18 shows a non-limiting, exemplary of UV HPLC chromatograms of root tissue extracts from 192-hour control hairy root line U-D2.

FIG. 19 shows non-limiting, exemplary mass spectra of compounds found in the Root 192 hour control of FIG. 18.

FIG. 20 shows a non-limiting, exemplary of UV HPLC chromatograms of root tissue extracts from 192-hour elicited hairy root line U-D2.

FIG. 21 shows non-limiting, exemplary mass spectra of compounds found in the Root 192 hour elicitor sample of FIG. 20.

FIG. 22 shows a non-limiting, exemplary of UV HPLC chromatograms of root tissue extracts from hairy root line U-D2 elicited with methyl jasmonate for 192 hours.

FIG. 23 shows a non-limiting, exemplary of UV HPLC chromatograms of root tissue extracts from hairy root line U-D2 elicited with hydrogen peroxide for 192 hours.

FIG. 24 shows a non-limiting, exemplary of UV HPLC chromatograms of medium extracts from 192-h elicited normal roots.

FIG. 25 shows a non-limiting, exemplary of UV HPLC chromatograms of root tissue extracts from 192-h elicited normal roots.

FIG. 26 shows non-limiting, exemplary mass spectra of compounds found in FIG. 25. Mass spectra in negative and positive modes are shown for each compound.

FIG. 27 shows a non-limiting, exemplary of UV HPLC chromatograms of medium extracts from 48-h elicited normal root cultures.

FIG. 28 shows non-limiting, exemplary mass spectra of compounds found in FIG. 27. Mass spectra in negative and positive modes are shown.

FIG. 29 shows a non-limiting, exemplary of UV HPLC chromatograms of medium extracts from 192-h elicited hairy root cultures of white mulberry line U-D2.

FIG. 30 shows non-limiting, exemplary mass spectra of compounds found in FIG. 29.

FIG. 31 shows a non-limiting, exemplary of UV HPLC chromatograms of medium extracts from 48-h elicited hairy root cultures of white mulberry line U-D2.

FIG. 32 shows chemical structures of the compound targeted/identified in white mulberry hairy root cultures.

FIG. 33 shows images of the establishment of white mulberry (Morus alba var. tatarica; source Ukraine) hairy root cultures. (Panel A) Seed of white mulberry. (Panel B) Two-week-old seedling. (Panel C) Eight-week-old seedling. (Panel D) Hairy root development from leaf infected with Agrobacterium rhizogenes. (Panel E) Growth of hairy roots on semi-solid medium and selection based on growth. (Panel F) Three selected lines after grown in liquid medium for 25 days. (Panel G) PCR analysis of selected hairy root lines. Genomic DNA was isolated from hairy root line U-A2, U-DI and U-D2. Analyses were performed with primers targeting rolC, aux1 and virD2 genes. Plasmid pRi15834 DNA was used as positive control. ddH2O was used as negative control.

FIG. 34 shows images of the phenotype of hairy root line U-D2 upon treatment with different elicitors for 192 hours.

FIG. 35 shows HPLC chromatograms of extracts from the medium and root of hairy root cultures of white mulberry line U-D2 after 192 h of elicitation treatment. The extracts of medium were concentrated 10-fold before HPLC analysis, while the roots were extract by 95% ethanol (40 mg dry root: 2 mL 95% ethanol). All chromatograms were monitored at 320 nm with same scale-100 mAU to 2500 mAU.

FIG. 36 shows UV-spectrum of the compounds identified in white mulberry hairy root system. (Panel A) HPLC chromatograms of U-D2 elicited medium (CD+MgCl₂+H₂O₂) extract after 48 hours treatment. (Panel B) UV-spectrum of the identified compounds inside medium samples. (Panel C) HPLC chromatograms of U-D2 elicited and control root tissue extract. (Panel D) (Panel B) UV-spectrum of the identified compound in root samples.

FIG. 37 shows graphs of yield of stilbenes and aryl benzofuran in the medium and root of white mulberry hairy root line U-D2 after treatment with different of elicitors. (Panel A) Yields expressed in mg/L of culture medium, (Panel B) Yields expressed in mg/g DW of root tissue. (Panel C) Yield of mulberroside A in the hairy root tissue after 192 h of treatment with multiple elicitor treatments. Values are the average of four biological replicates and error bars represent standard deviation.

FIG. 38 shows non-limiting, exemplary ranges of concentrations of compounds in hairy root and normal root cultures

FIG. 39 shows chemical structures of stilbenes and benzofurans identified in hairy root cultures of white mulberry.

FIG. 40 shows HPLC chromatograms of extracts from the medium of white mulberry line U-D2 upon different elicitation treatments at 192 hours. (A). Non-treated control. (B) CD+MgCl₂+MeJA+H₂O₂. (C). CD+MgCl₂+MeJA. (D) CD+MgCl₂+H₂O₂. All chromatograms were monitored at 320 nm.

FIG. 41 shows UV-spectra of compounds identified in white mulberry hairy root cultures. (A) HPLC chromatogram of hairy root line U-D2 medium extract after 48 hours of elicitation with CD+MgCl₂+H₂O₂. (B) UV-spectrum of the compounds identified in extracts of the culture medium. (C) HPLC chromatogram of hairy root line U-D2 tissue extract at 320 nm. (D) UV-spectrum of the identified compound in the root extract.

FIG. 42 shows characterization of 3′-prenylresveratrol. (A) HPLC chromatograms (UV 320 nm) of ethyl acetate extraction of U-D2 elicited medium (red line) and arachidin-2 standard (blue line). The UV-spectra and chemical structures of 3′-prenyl-resveratrol and arachidin-2 standard are shown. (B) HPLC-PDA-ESI-MS3 analysis of 3′-prenylresveratrol. (C) HPLC-PDA-ESI-MS3 fragmentation pattern of 3′-prenylresveratrol and arachidin-2.

FIG. 43 shows characterization of chalcomoracin. (A) HPLC chromatogram (UV 320 nm) of ethyl acetate extract of the medium of white mulberry hairy root line U-D2. Chemical structure of chalcomoracin and, without wishing to be bound by theory, fragmentation pattern of peak identified as chalcomoracin are shown. (B) HPLC-PDA-ESI-MS3 analysis of chalcomoracin. (C) HPLC-PDA-MS3 fragmentation pattern of chalcomoracin.

FIG. 44 shows HPLC analyses of the medium extract from elicited hairy root cultures of white mulberry (CD+MgCl₂+H₂O₂, 144 h) and arachidin-2 standard. Samples were analyzed using conditions optimized for peanut stilbenes

FIG. 45 shows quantification of HPLC peak area of chalcomoracin detected in white mulberry hairy root line U-D2.

FIG. 46 shows white mulberry line U-D2-hairy root inoculum. In embodiments, inocula can be 5 hairy root branches (˜5 cm) from 25-35 day hairy root cultures. Inoculum is placed into 500 mL flask with 100 mL of full strength MVS medium.

FIG. 47 shows growth comparison of 3 hairy root branches and 5 hairy root branches. In embodiments, culture conditions can include an inoculum of 5 hairy root branches per flask. Inoculum is placed into 500 mL flask with 100 mL of full strength MVS medium.

FIG. 48 shows growth comparison of 3 hairy root branches and 5 hairy root branches. In embodiments, culture conditions can include an inoculum of 5 hairy root branches per flask. Inoculum can be placed into 500 mL flask with 100 mL of full strength MVS medium. Medium is replaced at week 4 or week 5 with fresh medium.

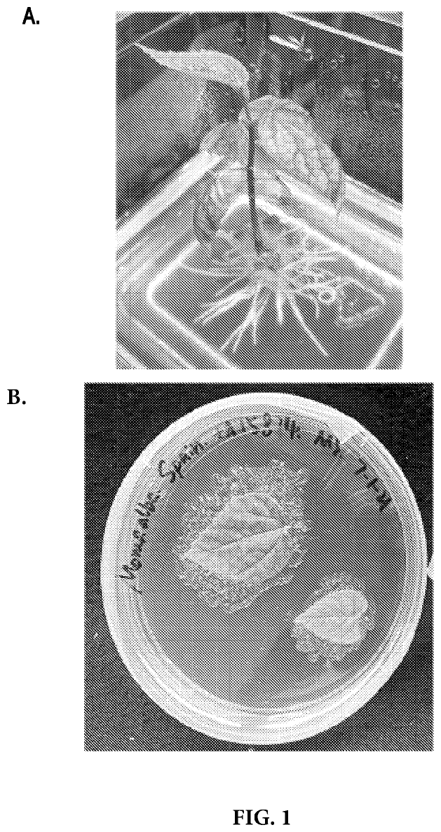

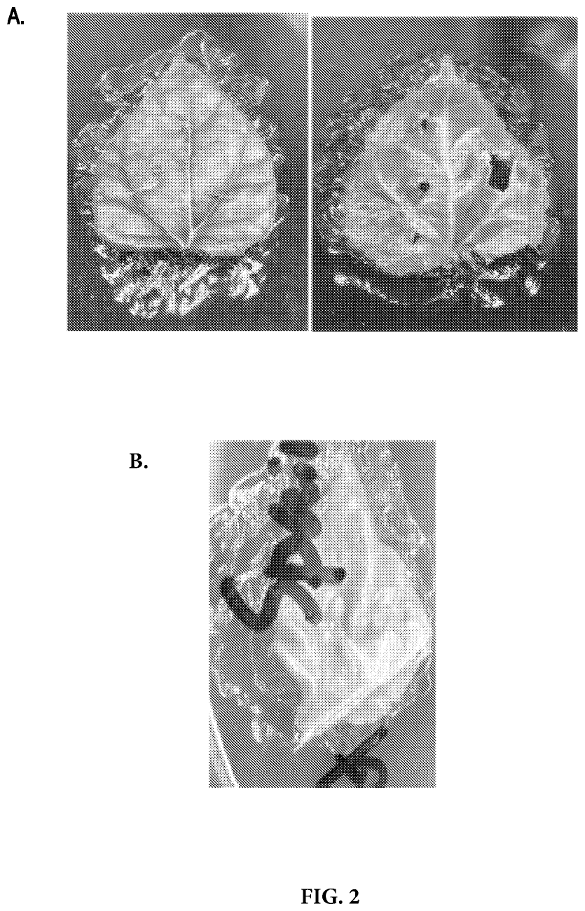

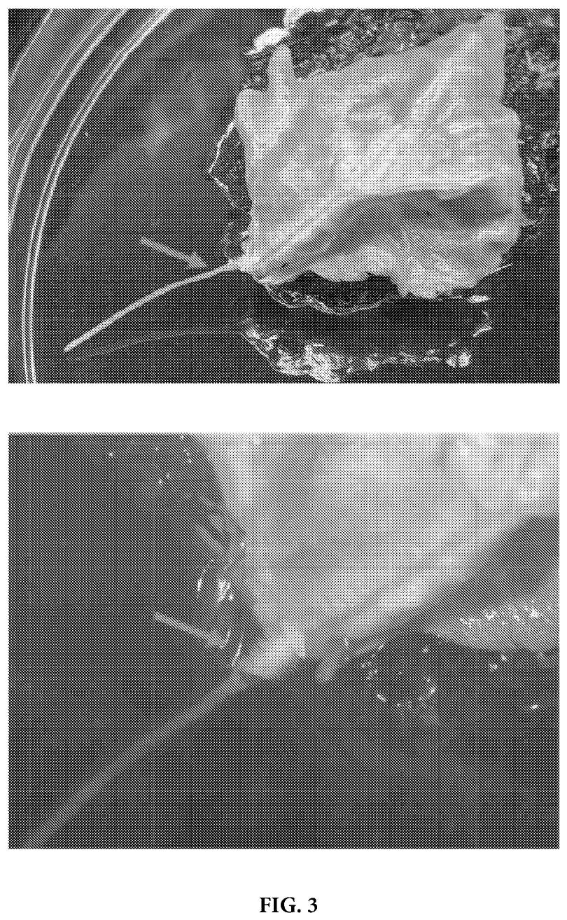

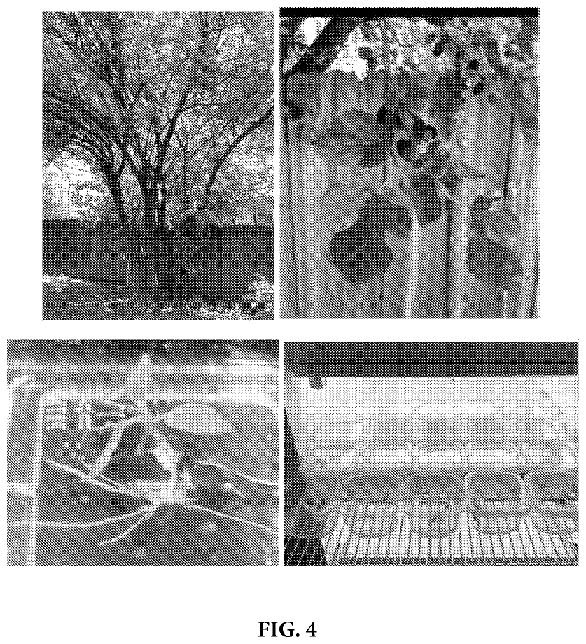

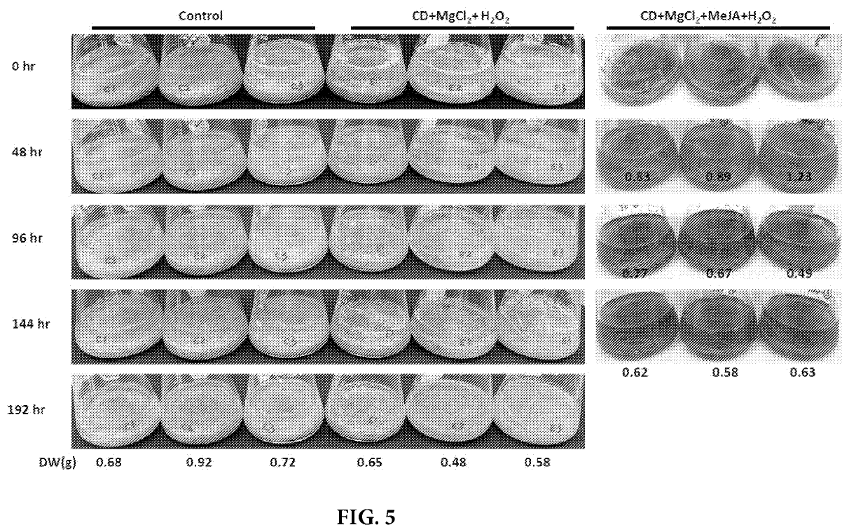

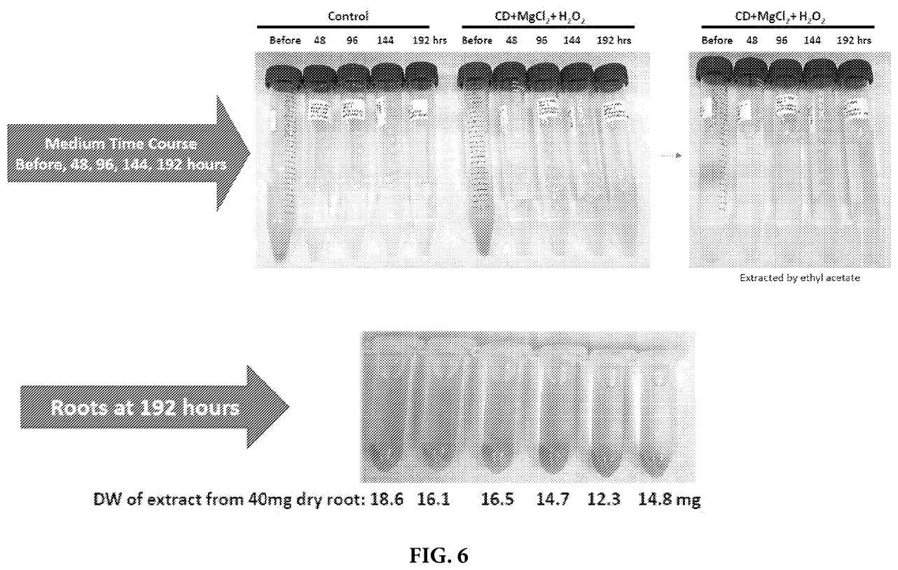

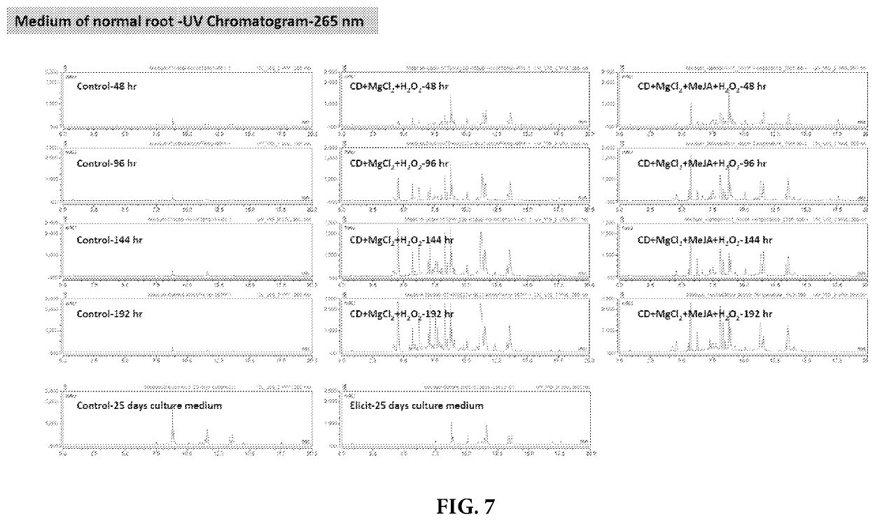

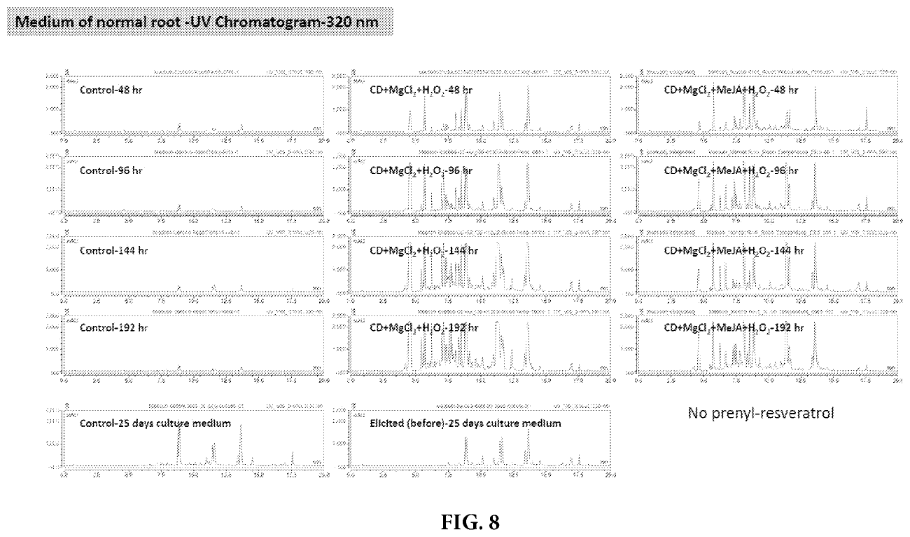

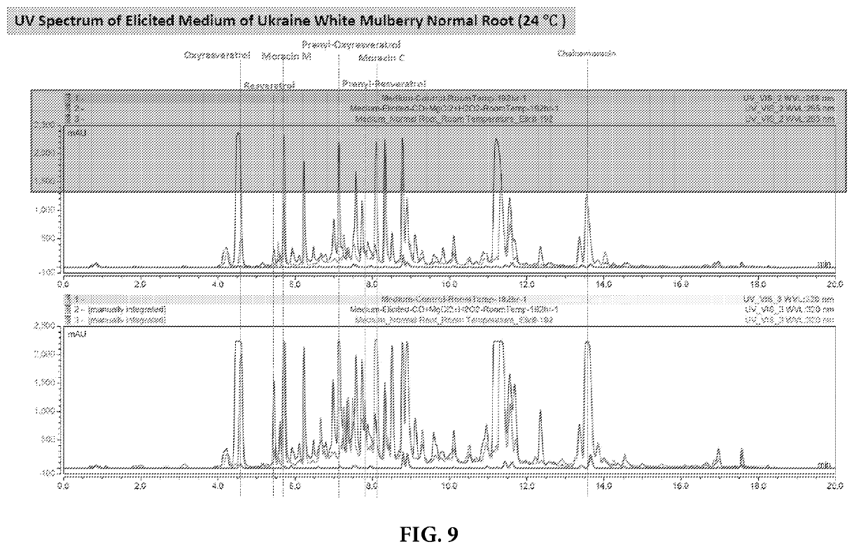

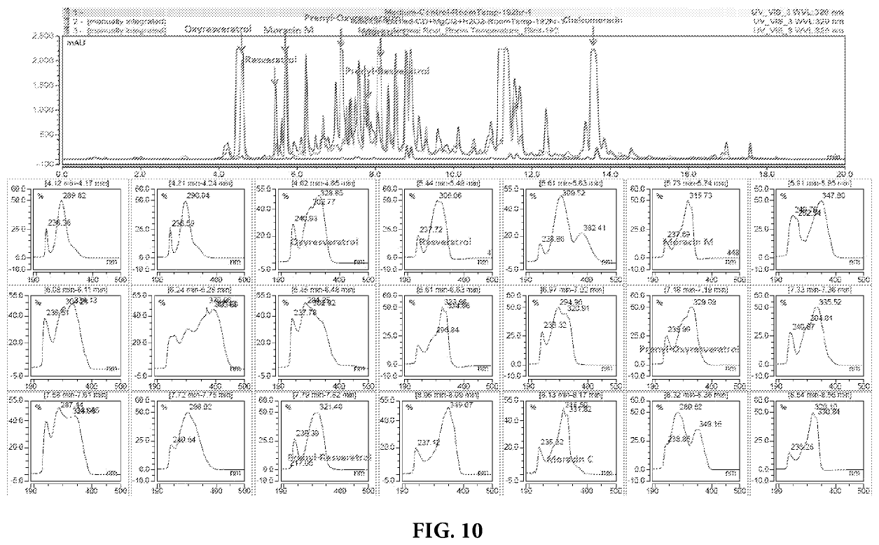

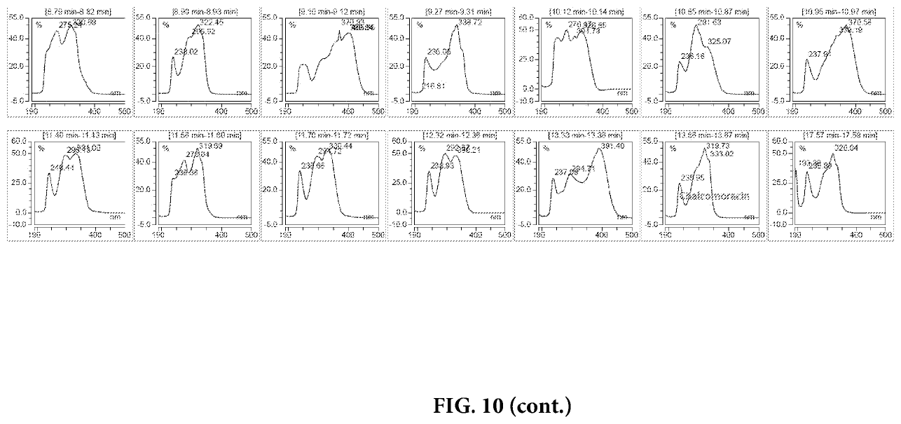

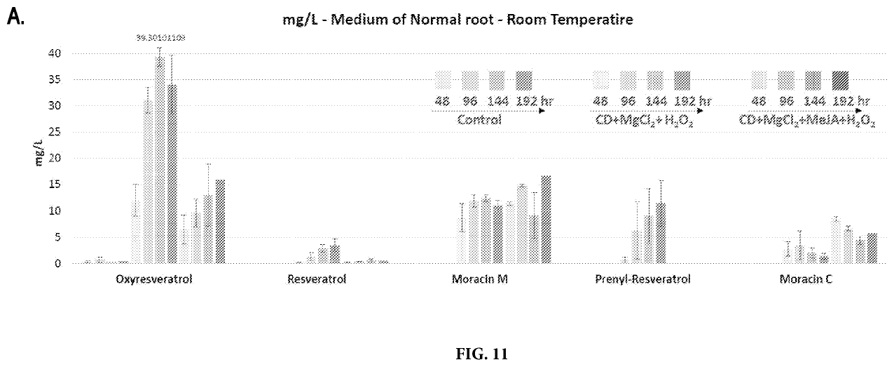

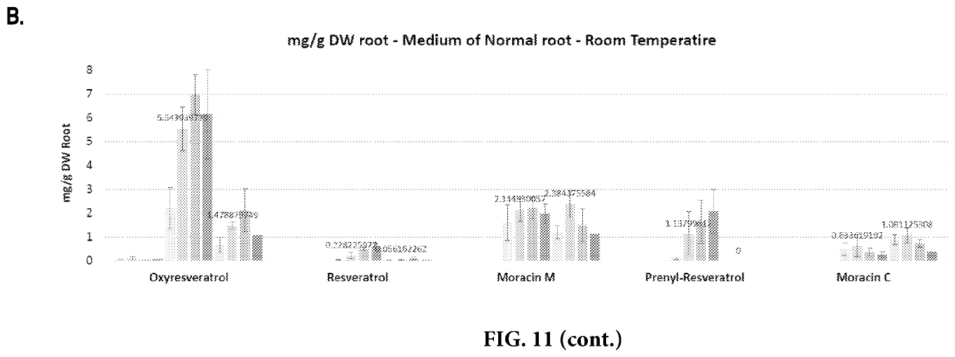

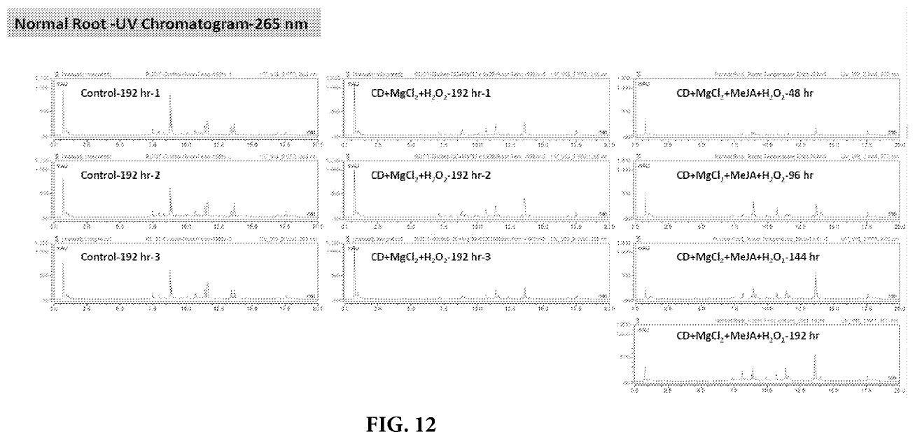

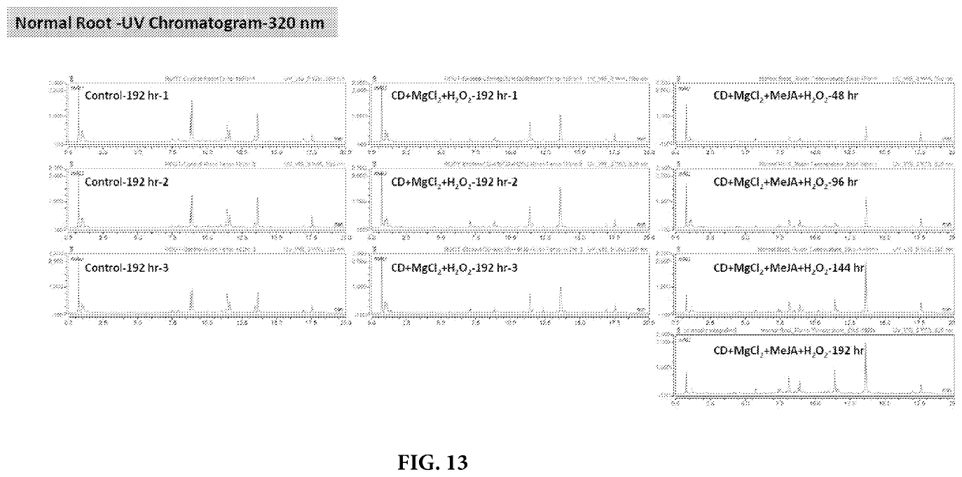

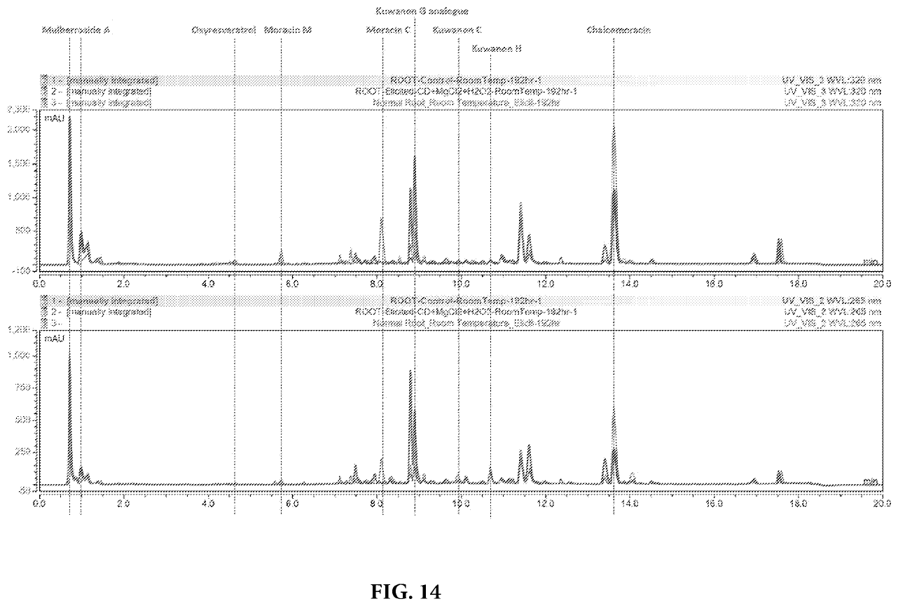

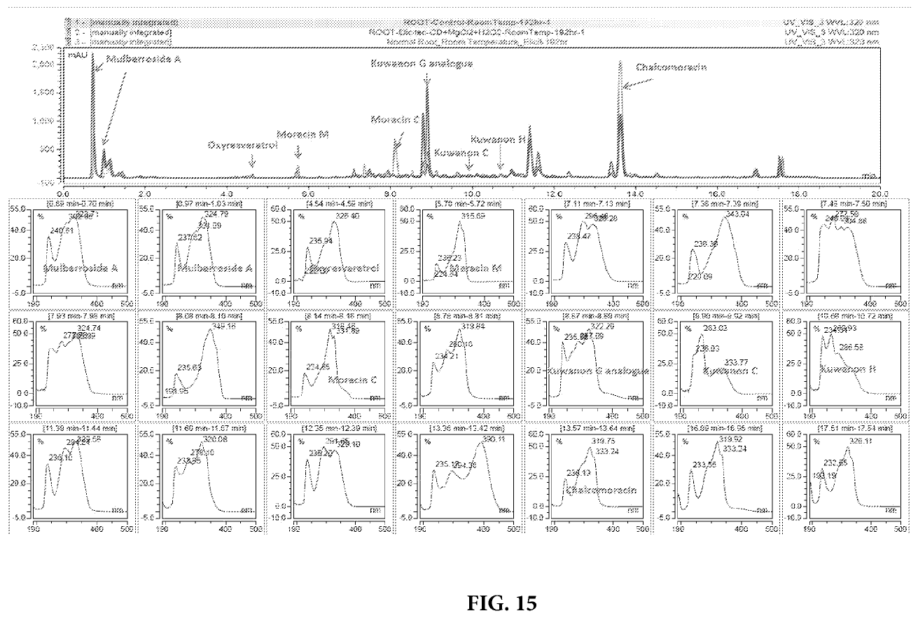

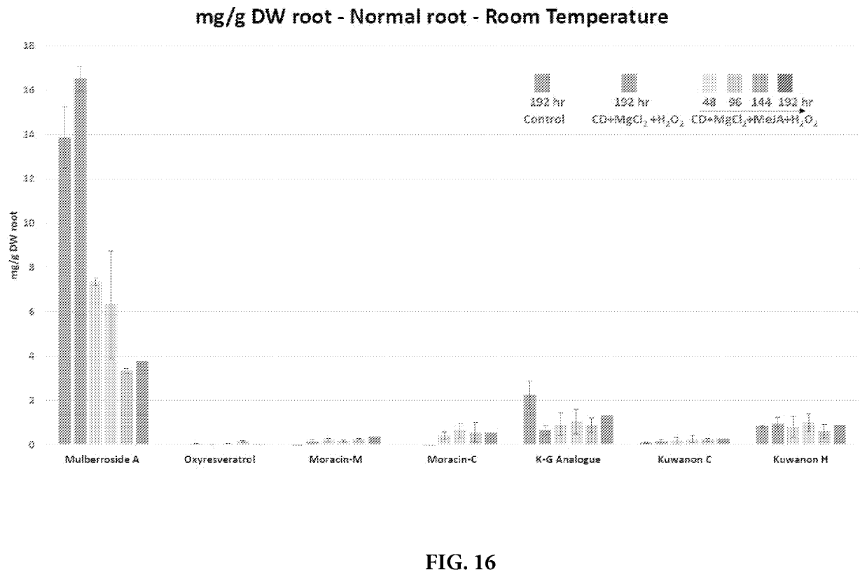

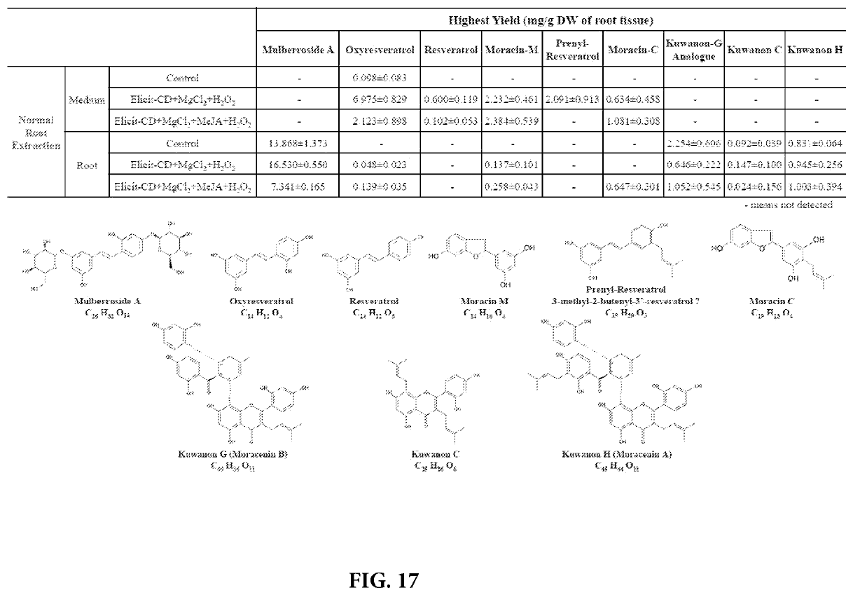

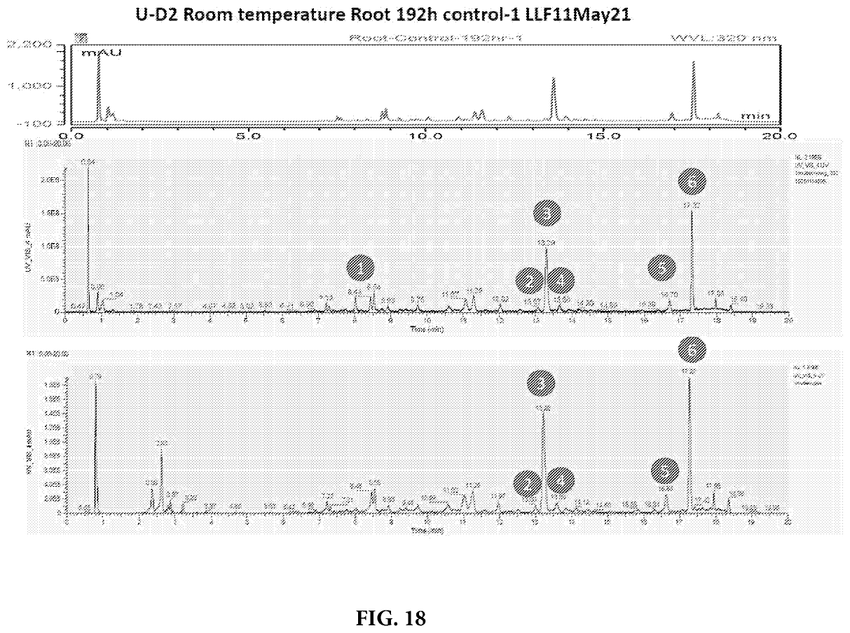

### DETAILED DESCRIPTION OF THE INVENTION

Detailed descriptions of one or more embodiments are provided herein. However, the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn towards botanical compositions and methods for producing a botanical composition. Aspects of the invention are drawn to botanical compositions, such as those comprising plant or botanical extracts from a Morus alba plant.

“Botanical” can refer to a material that is or can be obtained from a tree-, plant-, weed- or herb-derived. As used herein, “botanically derived” can refer to a material capable of having been derived from a botanical, as by isolation or extraction; however, “botanically derived” is not limited in this application to materials which actually are isolated or extracted from a botanical, but also includes materials obtained commercially or synthetically.

The term “botanical composition” can refer to a composition that contains vegetable matter, which can derive from any part of a plant or plant material, or combinations thereof. Depending in part on its intended use, a botanical composition can be a food, drug, medical food, a cosmetic component, dietary supplement, medicinal supplement, nutritional supplement, nutritional or nutrient. The botanical composition can include a number of additional agents.

In embodiments, the botanical composition can comprise a combination of plant materials (i.e., a combination of plant extracts or plant tissues). For example, such “combination composition” can refer to a botanical composition which comprises a mixture of at least two different plant materials. In embodiments, the at least two different plant materials can be from the same plant (such as a mixture comprising a first material from an aerial part of the plant and a second material from the root of the plant). In other embodiments, the at least two different plant materials can be from different plants (such as a mixture comprising a first material from a first plant, and a second material from a second plant). For example, the plant materials can be plant extracts, as described herein. In embodiments, the combination composition can comprise a mixture of plant materials from two or more of the plant species.

In embodiments, the extract can comprise extracts from various parts of a plant or plant tissue. As used herein, the term “plant tissue” can refer to a collection of similar cells performing an organized function for the plant. For example, plant tissue can refer to the tissue of any organism of the plant kingdom. In embodiments, the plant tissue can be any portion or portions of the plant. For example, plant tissue can refer to bark, roots, leaves, flowers, needles, bulbs, berries, rhizomes, rootstocks, stems, seeds, and/or the entire plant. In certain embodiments, the plant tissue comprises root tissue. For example, the plant tissue can refer to the “aerial” part of the plant, such as the phyllosphere. For example, the phyllosphere can comprise the caulosphere (stems), phylloplane (leaves), anthosphere (flowers), aerial roots, and carposphere (fruits).

In embodiments, the plant tissue can be used to produce a plant tissue culture. As used herein, the term “plant tissue culture” and “plant culture” can be used interchangeably. As used herein, the term “plant tissue culture” can refer to the product of culturing plant seeds, organs, explants, tissues, cells, or protoplasts on a chemically defined nutrient media. For example, the plant tissue culture can comprise root tissue culture. As used herein, the terms “root culture” and “root tissue culture” can be used interchangeably. For example, a root tissue culture can be produced from a plant tissue culture from root tissue.

The botanical extracts prepared according to the invention can be obtained by means of producing a root culture from plant tissue of Morus alba.

The term “root culture” can refer to a plant tissue culture derived from the roots of the plant. For example, root culture can comprise root tissue, medium of said root culture, or a combination thereof. For example, the root culture can be a culture of aseptically germinated seeds in medium that can be induced to grow.

The term “root tissue” can refer to plant tissue that comprises the roots. For example, the root tissue can comprise the epidermis, the cortex, and the vascular cylinder.

In embodiments, the root culture comprises a hairy root culture. A “hairy root culture” can refer to plant tissue that comprises the roots transformed by bacteria that contain root-inducing plasmids. As used herein, the term “hairy root culture” and “transformed root culture” can be used interchangeably. For example, the hairy root culture can be produced by infecting a plant with an Agrobacterium rhizogene bacteria. As used herein, the terms “Agrobacterium rhizogene” and “Rhizobium rhizogene” can be used interchangeably. In embodiments, the hairy root culture can result in a genetically transformed root culture that can produce increased levels of metabolites. For example, the metabolites can comprise secondary metabolites.

In embodiments, the hairy root culture can be produced by infecting a leave of Morus alba with Agrobacterium rhizogenes. The production can further comprise isolating genomic data from the hairy root line.

In embodiments, the root culture is a not a hairy root culture.

In embodiments, the botanical composition is produced from a Morus alba plant. As used herein, the terms “Morus alba”, “white mulberry”, “common mulberry”, and “silkworm mulberry” can be used interchangeably. Morus alba is a species from the Moraceae Family, which further comprises various subspecies such as Morus alba var. alba, Morus alba var. artropurpurea, Morus alba var. bungeana, Morus alba var. emarginata, Morus alba var. indica, Morus alba var. laevigata, Morus alba var. latifolia, Morus alba var. mongolica, Morus alba var. multicaulis, Morus alba var. nigriformis, Morus alba var. serrata, Morus alba var. skeletonia, Morus alba var. stylosa, and Morus alba var. tatarica.

In embodiments, the root culture can comprise a non-hairy root culture.

In embodiments, the plant tissue culture comprises a culture medium. As used herein, the terms “media” and “medium” can be used interchangeably. As used herein, the term “medium” can refer to a solution for growth, viability, or storage of a culture. In embodiments, the plant tissue can be cultured in the media for a period of time to produce a root culture. In embodiments, the culture medium can be “MSV” culture medium. See, for example, Condori, J.; Sivakumar, G.; Hubstenberger, J.; Dolan, M. C.; Sobolev, V. S.; Medina-Bolivar, L. F. Induced biosynthesis of resveratrol and the prenylated stilbenoids arachidin-1 and arachidin-3 in hairy root cultures of peanut: Effects of culture medium and growth stage. Plant Physiol. Biochem. 2010, 48, 310-318.

In embodiments, the root culture can be incubated in an elicitation medium for a period of time. An elicitation medium can refer to a medium that comprises elicitors. In embodiments, the elicitors can increase the production of metabolites. For example, the metabolites can comprise secondary metabolites.

In embodiments, the plant tissue can be cultured for a period of time in a culture medium. A “culture medium” can refer to a medium for growing, storing, handling, and/or maintaining cells and cell lines.

The term “culture” or “culturing” can refer to the growth of any cell, organism, multicellular entity, or tissue in a medium, or the act of producing the same. As used herein, the period of time the plant tissue is cultured in the culture medium comprises about 1 min, about 5 min, about 10 min, about 20 min, about 30 min, about 60 min, about 2 hours, about 5 hours, about 12 hours, 1 day, about 5 days, about 10 days, about 20 days, about 25 days, about 30 days, about 1 month, about 2 months, about 3 months, about 6 months, about 1 year, or more than 1 year.

In embodiments, the elicitation medium can comprise one or more elicitors. As used herein, the term “elicitor” can refer to a natural or synthetic compound which can stimulate the production of certain compounds. For example, an elicitor can increase the production of a botanical composition.

In embodiments, the tissue culture can be incubated with the one or more elicitors for a period of time comprising about 1 min, about 5 min, about 10 min, about 20 min, about 30 min, about 60 min, about 2 hours, about 4 hours, about 5 hours, about 12 hours, about 24 hours, about 48 hours, about 96 hours, about 192 hours, about 5 days, about 10 days, about 20 days, about 25 days, about 30 days, about 1 month, about 2 months, about 3 months, about 6 months, about 1 year, or more than 1 year.

Elicitors can be classified based on their ‘nature’ as abiotic elicitors or biotic elicitors or based on their ‘origin’ such as exogenous elicitors and endogenous elicitors 3. See Table 1 of Halder, Mihir, Sayantika Sarkar, and Sumita Jha. “Elicitation: A biotechnological tool for enhanced production of secondary metabolites in hairy root cultures.” Engineering in life sciences 19.12 (2019): 880-895. Biotic elicitors can be either crude extracts or partially purified products derived from either pathogen (fungal, bacterial, yeast) or the plant itself. They can be either of defined composition such as polysaccharides, glycoproteins, inactivated enzymes, purified chitosan (CHI), pectin, chitin, alginate, curdlan, xanthan, elicitin etc. or of complex composition such as yeast extract (YE) and fungal homogenate. Abiotic elicitors can include various chemical and physical stressors such as light and UV-radiation; salts of heavy metals (for example, Ag2S2O3, AgNO3, CdC12, CuC12, CuSO4, VOSO4, NiSO4, selenium); temperature shift; osmotic stress induced by mannitol, sorbitol, sodium chloride, potassium chloride, cadmium chloride, PVP, and the like; intracellular signaling molecules such as jasmonic acid (JA), methyl jasmonate (MJ), salicylic acid (SA), acetyl salicylic acid (ASA), and systemin.

In embodiments, the one or more elicitors can comprise those described herein. For example, the one or more elicitors can comprise cyclodextrin, magnesium chloride, methyl jasmonate, hydrogen peroxide, and their derivatives (such as methylated forms). For example, the cyclodextrin can comprise α-cyclodextrin, β-cyclodextrin, and γ-cyclodextrin. For example, the cyclodextrin can be a methylated β-cyclodextrin, non-limiting examples of which comprise 2-O-methylated-β-cyclodextrin; 2,6-O-dimethyl-β-cyclodextrin; 2, 3, 6-O-trimethyl-4 types of β-cyclodextrin; fully methylated-β-cyclodextrin; randomly methylated-β-cyclodextrin; or partially methylated-β-cyclodextrin.

In addition to cyclodextrin other elicitors can include chitosan. See, for example, Halder, Mihir, Sayantika Sarkar, and Sumita Jha. “Elicitation: A biotechnological tool for enhanced production of secondary metabolites in hairy root cultures.” Engineering in life sciences 19.12 (2019): 880-895.

In embodiments the cyclodextrin can be present in a concentration of about 1 mg/L, about 5 mg/L, about 10 mg/L, about 20 mg/L, about 50 mg/L, about 100 mg/L, about 500 mg/L, about 1 g/L, about 18 g/L, about 20 g/L, about 50 g/L, about 100 g/L, about 250 g/L, about 500 g/L, or about 1 kg/L.

In embodiments, the magnesium chloride can be present in a concentration of about 1 μM, about 10 μM, about 50 μM, about 100 μM, about 250 μM, about 500 μM, about 1 mM, about 5 mM, about 10 mM, about 25 mM, about 50 mM, about 100 mM, about 250 mM, about 500 mM, about 1 M, about 5 M, about 10 M, or about 100 M.

In embodiments, the hydrogen peroxide can be present in a concentration of about 1 μM, about 10 μM, about 50 μM, about 100 μM, about 250 μM, about 500 μM, about 1 mM, about 5 mM, about 10 mM, about 25 mM, about 50 mM, about 100 mM, about 250 mM, about 500 mM, about 1 M, about 5 M, about 10 M, or about 100 M.

In embodiments, the botanical composition is produced from a Morus alba plant. As used herein, the terms “Morus alba”, “white mulberry”, “common mulberry”, and “silkworm mulberry” can be used interchangeably. Morus alba is a species from the Moraceae Family, which further comprises two subspecies of Morus alba var. alba and Morus alba var. multicaulis.

The term “botanical extract” or “plant extract” can refer to a product prepared by separating, by chemical or physical process, one portion of a plant from another. For example, a product prepared by separating, by chemical or physical process, any medicinally active portions of a plant from the inactive or inert components.

In embodiments, the botanical composition can be isolated or extracted from the root culture, thereby providing a botanical extract. The term “isolate” can refer to a component that is isolated from its whole. The term “extract” can refer to a substance made by extracting a part of a raw material. For example, the extract can comprise a plant tissue extract. For example, the root culture can be contacted with a non-polar solvent and the organic layer extracted from the aqueous layer. For example, the root culture can be contacted with a polar solvent and the organic layer extracted from the aqueous layer. For example, the root culture can be contacted with a polar solvent miscible with water and the soluble metabolites extracted from the root culture.

In embodiments, the method of producing the botanical extract can further comprise contact a plant culture described herein with a solvent. The term “solvent” can refer to a substance that which dissolves, suspends, or extracts reactants. In embodiments, the solvent can comprise a polar solvent or a non-polar solvent. In embodiments, the solvent can comprise a protic solvent or an aprotic solvent.

In embodiments, the botanical extracts can be provided by the use of a polar solvent. The term “polar solvent” can refer to a solvent that comprises dipole moments. For example, a polar solvent can be miscible with water and polar solvents. For example, a polar solvent can comprise chemical species in which the distribution of electrons between covalently bonded atoms is not even. For example, the polarity of solvents can be assessed by measuring any parameter known to those of skill in the art, including dielectric constant, polarity index, and dipole moment (see, e.g., Przybytec (1980) “High Purity Solvent Guide,” Burdick and Jackson Laboratories, Inc.). The polar extracts of the invention can comprise any percentage of polar solvent including, but not limited to, for example 1-10% polar solvent, 10-20% polar solvent, 20-30% polar solvent, 30-40% polar solvent, 40-50% polar solvent, 50-60% polar solvent, 70-80% polar solvent, 80-90% polar solvent and 90-100% polar solvent. Examples of polar solvents include but are not limited to ethyl alcohol (ethanol), butyl alcohol (butanol), methanol, water, acetic acid, tetrahydrofuran, ethylene glycol, propylene glycol, butylene glycol, pentylene glycol, hexylene glycol, pentanol, hexanol, butanediol, pentanediol, hexanediol, octanediol, glycerl, N,N-dimethylformamide, dichloromethane, acetonitrile, dimethylformamide, dimethyl sulfoxide, acetone, or n-propanol.

In other embodiments, the plant extracts can be provided by the use of a non-polar solvent (i.e., non-polar extract). As used herein, “nonpolar” and “non-polar” can be used interchangeably. As used herein, the term “nonpolar solvent” can refer to a solvent comprising molecules that do not have an overall dipole. For example, the solvent comprises molecules comprising bonds between atoms with similar electronegativities (e.g., a carbon-hydrogen bond). For example, the nonpolar molecule comprises equal sharing of electrons between atoms or the arrangement of polar bonds leads to overall no net molecular dipole moment. The non-polar extracts of the invention can comprise any percentage of non-polar solvent, including but not limited to, for example, 1-10% non-polar solvent, 10-20% non-polar solvent, 20-30% non-polar solvent, 30-40% non-polar solvent, 40-50% non-polar solvent, 50-60% non-polar solvent, 70-80% non-polar solvent, 80-90% non-polar solvent, and 90-100% non-polar solvent. Examples of non-polar solvents include but are not limited to isooctane, hexane, pentane, benzene, chloroform, diethyl ether, ethyl acetate, hydrocarbons, cyclohexane, toluene, or 1,4-dioxane.

Hydrophobic molecules can be non-polar and thus can interact with (e.g., associate, aggregate, etc.) other neutral molecules and non-polar solvents. For example, nonpolar or hydrophobic molecules can interact through non-covalent interactions. For example, the non-covalent interaction is a van der Waals interaction. For example, the van der Waals interaction are London forces. Hydrophilic molecules can be polar and dissolve by water and other polar substances.

In embodiments, the botanical extract can be produced by contacting the plant tissue cultures described herein with the solvent and extracting the organic layer from the aqueous layer.

In embodiments, when the botanical extract is collected, the liquid can be further processed if need be (such as by a concentrating column and the like), active ingredients can be separated from this extract via affinity chromatography, mass chromatography and the like.

The term “aqueous extract” can refer to a plant extract where the extraction has been performed using water as the only solvent. The term “organic extract” can refer to a plant extract where the extraction has been performed using an organic solvent.

In embodiments, the botanical composition can comprise a plant extract prepared from an entire plant, or a plant extract prepared from a part of a plant, such as flowers, flowering tops, aerial parts, leaves, stems, buds, roots, bulbs, rhizomes, bark, seeds, fruit or fruit peel, bark, kernel, stones, berries, sap, resin, latex and thallus of the plant. In embodiments, the plant extract is prepared from the roots of the plant.

For example, the botanical composition can include a plant extract prepared from the root or aerial parts of the Morus alba plant.

In embodiments, the botanical composition produced by methods described herein can comprises one or more aryl benzofuran compounds. In embodiments, the aryl benzofuran compound is a compound comprising Formula (I):

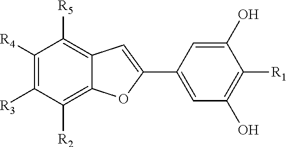

### Formula (I)

wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof.

For example, the compound of Formula (I) comprises:

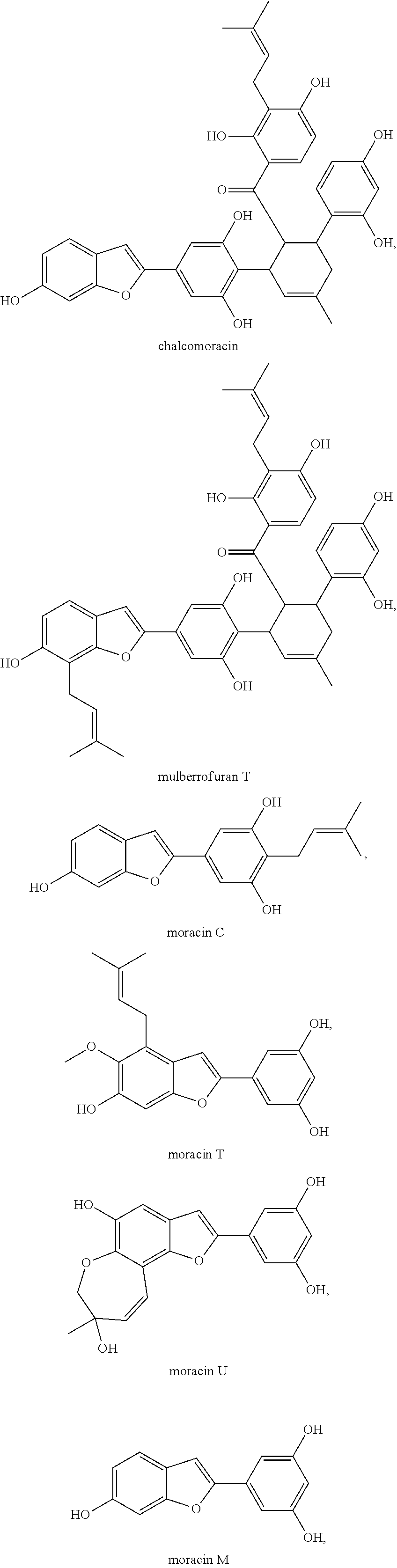

or a glucosidyl derivative thereof.

In embodiments, the aryl benzofuran compound or derivative thereof is present in a concentration of about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

For example, aspects of the invention are drawn towards producing moracin in a botanical composition. “Moracin” can refer to a compound isolated from Morus alba.

In embodiments, the botanical composition further comprises a stilbenoid, a flavone, a combination thereof, or a derivative thereof. For example, the stilbenoid or derivative thereof comprises resveratrol, oxyresveratrol, mulberroside, prenyl-resveratrol, prenyloxyresveratrol, or combination thereof.

In embodiments, the oxyresveratrol compound or derivative thereof comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments, the resveratrol compound or derivative thereof comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments, the prenyl-resveratrol compound or derivative thereof comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments the mulberroside A compound or derivative thereof comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments the prenyloxyresveratrol compound or derivative thereof comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments, the flavone or derivative thereof comprises kuwanon or a derivative thereof. For example, the kuwanon or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E.

In embodiments, kuwanon C comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments, kuwanon H comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

In embodiments, the kuwanon G analog comprises about 0.01 μg/g, about 0.025 μg/g, about 0.05 μg/g, about 0.1 μg/g, about 0.25 μg/g, about 0.5 μg/g, about 0.75 μg/g, about 1 mg/g, about 5 mg/g, about 10 mg/g, about 25 mg/g, about 35 mg/g, about 50 mg/g, about 75 mg/g, about 100 mg/g, about 250 mg/g, about 500 mg/g, about 750 mg/g, about 1 g/g per dry weight of root tissue.

Embodiments can comprise a fraction or an isolated component of a botanical composition as described herein. For example, a “fraction” can refer to a quantity collected from a batch of a substance in a fraction during a separation process. An “isolated component” can refer to a component of material that is separated from other components of the material.

In this way, the composition can be formulated for administration via several conventionally acceptable routes. For example, the botanical composition can be provided in a form selected from the group consisting of a cream, a serum, a liquid, a gummy, a tablet, a tincture, a pellet, a lozenge, a granule, a suspension, and a capsule.

In embodiments, the botanical composition can be prepared by mixing the active plant extracts with one or more inert carriers, excipients, or diluents (e.g., vehicles, binders,) suitable for the selected route of administration. The term “excipients” can refer to pharmaceutically or cosmetically acceptable organic or inorganic substances which do not deleteriously react with the active compounds. Examples of excipients include, but are not limited to, dietary suitable starch, vegetable oil, vegetable gums, gelatins, soy extracts, sugars, grains, natural and artificial flavorings, and the like. Examples of carriers include, but are not limited to, water, salt solutions, alcohol, plant seed and vegetable oils, glycols, gelatin, lactose, amylose, magnesium stearate, talc, silicic acid, viscous paraffin, perfume oil; fatty acid monoglycerides and diglycerides, fatty acid esters, hydroxymethylcellulose, and the like. The botanical compositions described herein can also be formulated as syrups and elixirs. Further, additional compositions can be readily prepared using technology which is known in the art such as described in detail in Remington's Pharmaceutical Sciences, Twentieth Edition.

The botanical compositions can be in a form suitable for oral use, for example, as tablets, troches, lozenges, pills, aqueous or oily suspensions, solutions, dispersible powders or granules, emulsions, hard or soft capsules, syrups or elixirs, pastes, gels or the like. Compositions intended for oral use can be prepared according to any known method, and such compositions can contain one or more agents selected from the group consisting of sweetening agents, flavoring agents, coloring agents, and preserving agents in order to provide commercially viable, pharmaceutically elegant and palatable compositions. Tablets can contain the active ingredient(s) in admixture with non-toxic pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They also can be coated for controlled delivery. For example, a “delayed release” dosage form releases a product or substance at a time other than promptly after administration. Examples of delayed-release systems include repeat-action tablets and capsules, and enteric-coated tablets where timed release is achieved by a barrier coating.

Aspects of the invention are drawn towards cosmetic compositions or pharmaceutical compositions, including dermatological compositions comprising the botanical compositions or the fractions or isolated components of a botanical composition described herein and methods of producing the same. A “cosmetic composition” can refer to compositions that can have a use in cosmetics and/or aesthetic applications, such as a non-therapeutic, non-pharmaceutical, use, that is to say a use intended for any area of skin and/or skin appendage referred to as healthy. An area of healthy skin refers to an area of skin on which the compositions according to the invention are applied, termed “non-pathological” by a dermatologist, also described as “normal”, that is to say which does not show any infection, scar, skin disease or ailment such as candidiasis, impetigo, psoriasis, eczema, acne or dermatitis, or any wounds or injuries and/or other dermatoses.

In embodiments, the cosmetic composition can comprise any botanical composition described herein and further comprise a cosmetically acceptable carrier, excipient, or diluent. In embodiments, the cosmetic composition can comprise one or more additives comprising solvents, thickeners, softeners, moisturizers, sealers, emollients, wetting agents, chelating agents, preservatives, anti-foaming agents, perfumes, and emulsifiers. In embodiments, the pharmaceutical composition can comprise any botanical composition described herein and further comprise a cosmetically acceptable carrier, excipient, or diluent. In embodiments, the pharmaceutical composition can comprise one or more additives comprising solvents, thickeners, softeners, moisturizers, sealers, emollients, wetting agents, chelating agents, preservatives, anti-foaming agents, perfumes, and emulsifiers.

In embodiments, the cosmetic composition can be formulated as an oil, a lotion, a gel, an emulsion, a balm, a stick, a milk, a cream, an ointment, a bar soap, or a dusting powder.

The terms “carrier oils” can refer to any lipid-based carrier materials, oil and/or aqueous solution, derived from any plant seed sources, suitable for administration of the active compounds in the botanical compositions. Carrier oils useful herein include any such materials known in the art that are nontoxic, have stand-alone beneficial and therapeutic effects, and do not interact with other components. The term “a pharmaceutically acceptable carrier” can refer to any substantially non-toxic carrier conventionally useable for administration in which the active composition of the invention and its derivatives will remain stable and bioavailable.

Botanical compositions can be formulated as oily suspensions in a pharmaceutically acceptable carrier such as a soft gelatin capsule whereby the active ingredient(s) is (are) mixed with an aqueous solution or oil medium, including suspending the active ingredient in a plant seed derived or vegetable oil, for example hemp seed oil, evening primrose seed oil, borage seed oil, olive oil, sesame oil or coconut oil. The oily suspensions can contain a thickening agent or other agent necessary to produce a commercially viable product, such as, beeswax, paraffin, lecithin, or cetyl alcohol. Sweetening and flavoring agents can be added to provide a palatable oral composition. These compositions can be preserved by the addition of an antioxidant such as ascorbic acid.

Compositions of the invention can be sterilized and/or mixed with auxiliary agents and other excipients including but not limited to other carrier oils, preservatives, glycerins, stabilizers, waxes, wetting agents, emulsifiers, suspending agents, lecithin, esters or partial esters, buffers, coloring agents, flavorings and/or aromatic substances and the like which do not deleteriously react with the active compounds.

The compositions described herein, including the botanical compositions, the cosmetic compositions, or the pharmaceutical compositions as described herein can be used in a method of decreasing the melanin content of human skin or hair, the method comprising applying to skin or hair an effective amount of a botanical composition, a cosmetic composition, or pharmaceutical composition as described herein.

The compositions described herein, including the botanical compositions, the cosmetic compositions, or the pharmaceutical compositions as described herein can be used in a method of modulating skin pigmentation or skin color in a subject, the method comprising applying to skin of the subject an effective amount of a botanical composition, a cosmetic composition, or pharmaceutical composition as described herein. In embodiments, modulating skin pigmentation or skin color can comprise treating a skin pigmentation disorder, wherein the skin pigmentation disorder comprises age spots, sun damage, lentigo or hyperpigmentation. In embodiments, modulating skin pigmentation or skin color can comprise whitening, lightening, brightening, or increasing radiance in at least a portion of skin of a subject. The composition can be applied to the skin or hair of a subject.

The compositions described herein, including the botanical compositions, the cosmetic compositions, or the pharmaceutical compositions as described herein can be used in a method for inhibiting or decreasing melanogenesis, for decreasing pigmentation of skin, for decreasing pigmentation of skin appendages, or for decreasing pigment spots on skin, such as brown pigment spots on skin. In embodiments, the methods can comprise orally administering an effective amount of a botanical composition, a cosmetic composition, or pharmaceutical composition as described herein. In embodiments, the methods can comprise topically administering an effective amount of a botanical composition, a cosmetic composition, or pharmaceutical composition as described herein. In embodiments, the botanical composition, the composition comprising said botanical composition, or the cosmetic composition or pharmaceutical composition can be administered topically onto skin or skin appendages of the animal. In embodiments, the botanical composition, the composition comprising said botanical composition, or the cosmetic composition or pharmaceutical composition can be applied topically onto all or part of the animal body. In embodiments, the animal can be a human. In embodiments, the botanical composition, the composition comprising said botanical composition, or the cosmetic composition or pharmaceutical composition can be applied topically onto at least one part of a human body selected from a hand, neck, neckline, stomach, arm, thigh, hip, waist, face, and combinations thereof.

The compositions described herein, including the botanical compositions, the cosmetic compositions, or the pharmaceutical compositions as described herein can be used in a method for treating a pathological condition including pigment dysregulation, comprising administering an effective amount of a botanical composition, a cosmetic composition, or pharmaceutical composition as described herein to an animal subject in need thereof. In embodiments, the subject can be a human subject. In embodiments, the pathological condition can be a pathological hyperpigmentation. In embodiments the pathological condition can be selected from Addison's disease, liver failure, purpura, melanoma, or dermatosis papulose nigra. In embodiments, the botanical composition, or the cosmetic composition or pharmaceutical composition can be administered topically.

In embodiments, the compositions can be intended to be administered orally, or intended to be applied to all or part of the body chosen from the hands, the neck, the neckline, the stomach, the arms, the thighs, the hips, the waist and/or the face, and/or the skin appendages, and preferentially the hands, the neck, the neckline and/or the face, more preferentially the face.

The term “skin appendages” can refer to the hair, the body hair, the nails, preferentially the hair and/or the body hair, more preferentially the body hair.

For the purposes of the present invention, decreasing the pigmentation of the skin and/or of the skin appendages includes decreasing the amount of melanin by at least 25%, by at least 50%, preferentially by at least 75%, more preferentially by at least 85% in the presence of a composition described herein, relative to the amount of melanin measured in the absence of the composition. The amount of melanin can be measured according to the techniques known by those skilled in the art, such as for example in B16 melanocytes or normal human epidermal melanocytes under conditions known in the art. In embodiments, the melanin measurement can be carried out by measuring the optical density at 475 nm.

In embodiments, the decrease in the pigmentation is long-lasting, that is to say that the level of pigmentation after application of a composition described herein is maintained for at least two days, preferentially at least four days after the use of the composition has been stopped.

Various in vivo measurement techniques can also be used to measure the decrease in pigmentation of the skin and/or of the skin appendages and in particular the persistence of this effect. The in vivo measurement can be carried out by chromametry. This technique measures the absorbance at distinct wavelengths (OD 420/520/620 nm) and allows a measurement of three parameters (L*, a* and b*) (L * represents the “clarity”, a* represents the “redness” and b* represents the “yellowness”).

The decrease in the amount of melanin in the skin and/or in the pigment spots can also be measured by a Siascope® (siascopy technique), generating a high-resolution spectrophotometric intracutaneous analysis and making it possible to measure in particular the total concentration of melanin in the epidermis.

For the purposes of the present invention, decreasing the pigment spots on the skin refers to a decrease in the pigmentation of the localized pigment spots, in particular in the number and/or the intensity of the pigment spots, typically on the areas of the face, neckline, neck, back, shoulders and/or hands, particularly spots on the back of the hands. In embodiments, the pigment spots in particular can be “brown spots” or “age spots” when they are located on a part of the body exposed to UV radiation and/or associated with chronological ageing or UV-induced ageing, for example during exposure to the sun. In embodiments, the pigment spots also include freckles, and/or post-inflammatory spots and/or spots which appear in response to attacks, and/or which are of hormonal origin, in particular in the context of a chloasma, and/or of drug-related origin, and/or for preventing and/or combating the unaesthetic pigment manifestations accompanying a pathological condition.

The compositions described herein can, in embodiments, be topically and/or orally acceptable cosmetic and/or nutraceutical or pharmaceutical, preferentially dermatological compositions. For the purposes of the present invention, the expression “topically and/or orally acceptable” can refer to a composition or ingredient which is suitable for application, respectively, topically and/or orally, which is, on average, or at least in non-sensitive or allergic individuals, non-toxic and non-irritant to the skin and does not induce an allergic response, and which is not chemically unstable.

The use of the compositions described herein may be oral and/or topical. In embodiments, it is used topically, for example, direct local application and/or vaporization of the extract on the surface of the skin.

The cosmetic compositions or pharmaceutical compositions can, respectively, also comprise at least one cosmetically acceptable or pharmaceutically acceptable excipient. In embodiments, the excipient(s) can be at least one of preserving agents, emollients, emulsifiers, surfactants, moisturizing agents, thickeners, texturing agents, film-forming agents, pigments, stabilizers, solubilizing agents, dyes and fragrances. In embodiments, the excipient(s) can be at least one of amino acids and derivatives thereof, polyglycerols, esters, polymers and cellulose derivatives, lanolin derivatives, phospholipids, lactoferrins, lactoperoxidases, sucrose-based stabilizing agents, vitamin E and derivatives thereof, xanthan gums, natural and synthetic waxes, plant oils, triglycerides, unsaponifiables, phytosterols, plant esters, silicones and derivatives thereof, protein hydrolysates, jojoba oil and derivatives thereof, liposoluble/water-soluble esters, betaines, aminoxides, plant extracts, saccharose esters, titanium dioxides, glycines, and parabens. In embodiments, the excipient(s) can be at least one of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerin, dihyclroxycetyl sodium phosphate, isopropyl hydroxycetyl ether, glycol stearate, triisononanoin, octyl cocoate, polyacrylamide, isoparaflin, laureth-7, a carbomer, propylene glycol, hexylene glycol, glycerol, bisabolol, a dimethicone, sodium hydroxide, PEG 30-dipolyhydroxystearate, capric/caprylic triglycerides, cetearyl octanoate, dibutyl adipate, grapeseed oil, jojoba oil, magnesium sulfate, EDTA, a cyclomethicone, xanthan gum, citric acid, sodium lauryl sulfate, mineral oils and waxes, isostearyl isostearate, propylene glycol dipelargonate, propylene glycol isostearate, PEG 8, beeswax, hydrogenated palm kernel oil glycerides, lanolin oil, sesame oil, cetyl lactate, lanolin alcohol, castor oil, titanium dioxide, lactose, saccharose, low-density polyethylene, and an isotonic saline solution. Combinations of any of the above excipients are also envisioned.

The compositions described herein can, in embodiments, be in the form of an aqueous or oily solution, a cream or an aqueous gel or an oily gel, in particular a shower gel, a milk, an emulsion, a microemulsion or a nanoemulsion, which is in particular oil-in-water or water-in-oil or multiple or silicone-based, mask, a serum, a lotion, a liquid soap, a dermatological bar, an ointment, a foam, a patch, an anhydrous product, which can, in embodiments, be preferably liquid, pasty, or solid. In embodiments, the cosmetic or pharmaceutical compositions described herein can be a cream or a serum.

In embodiments, the compositions described herein are intended to be applied, advantageously topically, to all or part of the body chosen from the hands, the neck, the neckline, the stomach, the arms, the thighs, the hips, the waist, and/or the face and/or the skin appendages, and preferentially the hands, the neck, the neckline and the face, more preferentially the face.

In another embodiment, botanical composition described herein is included in a nutraceutical composition also comprising at least one nutraceutically acceptable excipient, at a concentration of from 1×10^{31 4}% to 10% by weight, preferentially between 1×10⁻⁴% and 5% by weight, more preferentially between 1×10⁻³% and 3% by weight relative to the total weight of the composition, particularly between 0.001% and 0.1% weight relative to the total weight of the composition. The term “nutraceutical composition” can refer to a composition which can be administered orally as a nontherapeutic food supplement and which is in the form of gel capsules, capsules, a powder or a gel. In some embodiments, it is not a medicament. In embodiments, extract of the normal or hairy root tissue and/or culture medium can be used in a cosmetic composition at a concentration between 1×10⁻⁴% and 50% (w/w), preferably between 1×10⁻³% and 20% (w/w), more preferably between 0.01% and 10% (w/w), and even more preferably between 0.01% and 5% (w/w), relative to the total weight of the composition.

The cosmetic and/or nutraceutical and/pharmaceutical composition described herein can, in embodiments, contain one or more other cosmetic and/or nutraceutical and/or pharmaceutical active ingredients, resulting in a supplementary effect and/or an effect of synergy with the extracts, botanical compositions, or fractions described herein.

In embodiments, a composition described herein may contain one or more other depigmenting and/or lightening active agents and/or active agents intended for decreasing the pigment spots on the skin, such as niacinamide or vitamin B3, arbutin, azelaic acid, ascorbic acid or derivatives thereof, a combination of the Saxifrage sarmentosa, Psidium guajava and Garica papaya plant extracts sold by BASF Beauty Care Solutions France under the name Dermawhite™ WF, a combination of sulfites and Camellia sinensis, Scutellaria baicalensis, Cucumis sativus and Pyrus malus extracts, sold under the name Phytolight™ BG or a combination of a pea extract and of sucrose dilaurate sold under the name Actiwhite™, or else 4-hydroxyphenoxyacetic acid derivatives, in particular 2-(4-hyclroxyphenoxy) propionic acid sold under the name Radianskin™ by BASF Beauty Care Solutions France.

In addition, “anti-ageing” active agents may be further included in the compositions described herein, for example:

an agent which stimulates fibronectin synthesis, in particular a maize extract, such an extract being sold by BASF Beauty Care Solutions France under the name Deliner™ and the palmitoyl pentapeptide sold by the company Sederma under the tradename Matrixil®; an agent which stimulates the formation of elastic fibers, such as an Origanum majorana extract sold under the name Dermagenist™; an agent which stimulates the expression of perlecan and of dystroglycan in the extracellular matrix and/or in the epithelial basal membrane, for instance a Polygonum bistorta extract sold under the name Perlaura™ by BASF Beauty Care Solutions France; an agent for protecting extracellular matrix fibroblast growth factor (FGF2) against degradation thereof and/or denaturation thereof, in particular a Hibiscus abelmoscus extract as described in the patent application in the name of BASF Beauty Care Solutions France filed under number FR0654316 and sold by BASF Beauty Care Solutions France under the name Linefactor™ and/or an agent for stimulating fibroblast growth, for example a fermented soya extract containing peptides, known under the name Phytokine™ sold by BASF Beauty Care Solutions France and also described in patent application EP 1 119 344 B1 (Laboratoires Expanscience), and preferentially a combination of these two extracts; an agent which stimulates laminin synthesis, in particular a biotechnology-modified malt extract, such an extract being in particular sold by BASF Beauty Care Solutions France under the name Basaline™; an agent which stimulates hyaluronan synthase 2 (HAS2) expression and/or activity, such as the plant extracts described in patent application FR 2 893 252 A1 and in particular an aqueous extract of Galanga (Alpinia galanga) and sold by BASF Beauty Care Solutions France under the name Hyalufix™; an agent which stimulates lysyl oxidase like (LOXL) synthesis, such as a Geophila cordifolia extract and those described in patent application FR 2 855 968, and in particular a dill extract sold by BASF Beauty Care Solutions France under the name Lys′Lastine™; an agent which stimulates intracellular ATP synthesis, in particular an extract of the alga Laminaria digitata; a collagen-stimulating active agent such as retinal and/or vitamin C; an active agent which inhibits metalloproteinases (MMPs) such as more particularly MMPs 1, 2, 3, 9, such as retinoids and derivatives, oligopeptides and lipopeptides, lipoamino acids, the Argania spinosa leaf extract sold by BASF Beauty Care Solutions France SAS under the name Arganyl™; lycopene; isoflavones, quercetin, kaempferol, apigenin; a replumping agent, in particular the hyaluronic acid filling spheres sold by BASF Beauty Care Solutions France under the name Hyaluronic Filling Spheres™; an agent for increasing the expression of LOX for augmenting the architecture of the epidermis, for instance a Cichorium intybus extract sold under the name LOXAge™ by BASF Beauty Care Solutions France; an agent for increasing collagen deglycation and/or increasing the expression of type I collagen, such as a combination of a Salvia miltiorrhiza leaf extract and of niacin, sold by BASF Beauty Care Solutions France under the name CollRepair™; an agent which stimulates lumican and collagen synthesis, such as a synthetic acetyl Gin Asp Val His tetrapeptide sold by BASF Beauty Care Solutions France under the name Dermican™ and described in patent application WO 2005/120554 A1; an agent for protecting and stimulating elastin and collagen, such as the Manilkara multinervis leaf extract sold by BASF Beauty Care Solutions France under the name Elestan™ and the Eperua falcata root extract sold by BASF Beauty Care Solutions France under the name Eperuline™; agents which stimulate keratinocyte proliferation, which preferentially can be used in the composition according to the invention, in particular retinoids such as retinal and esters thereof, including retinyl palmitate, and phloroglucinol; agents which stimulate keratinocyte differentiation, for example minerals such as calcium and lignans such as secoisolariciresinol, and also the Achillea millefollium extract sold under the name Neurobiox™ by BASF Beauty Care Solutions France. In embodiments, a composition described herein may also contain one or more anti-ageing active ingredients.

In embodiments, a composition described herein may also contain one or more soothing agents, such as, for example, pentacyclic triterpenes, ursolic acid and salts thereof, oleanolic acid and salts thereof, betulinic acid and salts thereof, salicylic acid salts and in particular zinc salicylate, bisabolol, allantoin, omega-3 unsaturated oils, cortisone, hydrocortisone, indomethacin and betamethasone, anti-inflammatory active agents, and in particular those described in application FR 2 847 267, in particular the Pueraria lobata root extract sold under the name Inhipase™, Theobroma cacao extracts. The vasoprotector or vasodilator active ingredients which act on the microcirculation can be chosen from flavonoids, ruscogenins, nicotinates and essential oils.

Another aspect described herein a method comprising the topical application of a composition described herein, for decreasing the pigmentation of the skin and/or of the skin appendages, preferentially of the body hair, and/or decreasing the pigment spots on the skin.

In embodiments, the method comprises the topical application of a composition described herein to all or part of the body selected from the hands, the neck, the neckline, the stomach, the arms, the thighs, the hips, the waist and/or the face, and/or the skin appendages, and preferentially the hands, the neck, the neckline and/or the face.

In embodiments, the composition is applied at least once per day or at least twice per day, for a period of 30 days, or longer than 30 days, such as 56 days.

In another aspect, a composition described herein can be used for the pharmaceutical, preferentially dermatological, use thereof, preferentially topically, in the treatment of pathological conditions involving a pigment dysregulation, such as Addison's disease, liver failure, purpura, a melanoma or dermatosis papulosa nigra, which is described as pathological hyperpigmentation. In embodiments, the composition is a pharmaceutical or dermatological composition also comprising at least one pharmaceutically, preferentially dermatologically, acceptable excipient.

In embodiments, the botanical, cosmetic, pharmaceutical, or dermatological compositions can be formulations capable of lightening the skin without any danger to the user. Thus, the botanical or cosmetic or pharmaceutical, notably dermatological, compositions can be prepared to have a pH between about 5 and about 8.

The combinations of plant extracts into a botanical composition can produce a synergistic effect/action. “Synergy” can refer to an effect produced by a combination (e.g., of two or more plant extracts) that is greater than the expected additive effectives of the combination components. See, for example, Kim, Ji Hye, Tae In Kim, and Jin-Yeul Ma. “Synergistic effects of novel herbal decoctions from Panax ginseng and Morus alba on tyrosinase activity and melanogenesis in vitro.” Heliyon 8.2 (2022): e08866. In embodiments, “synergy” between two or more plant extracts can result in increased whitening activity. As used herein, “synergy” or “synergistic interactions” can refer to the interaction or cooperation of two or more plant extracts to produce a combined effect greater than the sum of their separate effects.

Aspects of the invention are also drawn to methods for preparing a botanical extract from a plant material. For example, the methods described herein can be used for preparing a botanical extract from a plant material from Morus alba.

### Methods of Extraction

In embodiments, the phrase “obtaining a plant material” can refer to any direct or indirect method of obtaining, securing, receiving, etc. a plant material. For example, the plant material can be fresh plant material, that has not been processed or has been minimally processed. In embodiments, the plant material comprises a plant seed. For example, the plant seed can be a plant seed of a Morus alba plant.

In embodiments, the plant seed is surface sterilized and germinated under aseptic conditions (see, e.g., F. Medina-Bolivar, J. Condori, A. M. Rimando, J. Hubstenberger, K. Shelton, S. F. O'Keefe, S. Bennett, M. C. Dolan, Production and secretion of resveratrol in hairy root cultures of peanut. Phytochemistry 68 (2007)). For example, the seeds of white mulberry are soaked in 50° C. warm water in a 50 mL conical tube overnight. The warm water is cooled down naturally. Any remaining fruit tissue outside the seeds is removed before starting the surface sterilization. Then the seeds are dipped into 70% ethanol for 3 min, followed by 66.7% Clorox (4% V/V NaOCl) with 0.05% tween-20 for 15 min. Afterward, the seeds are put back in 70% ethanol for 1 min and rinsed thoroughly with sterilized distilled water 4-5 times. The sterilized seeds are placed on plates containing MS (Murashige and Skoog; 1962) basal media with 30 g/L sucrose and 4 g/L phytagel and cultured at 24° C. under darkness until germination.

In embodiments, the seeds can be grown into seedlings wherein the leaves can be excised and inoculated with Agrobacterium rhizogenes to establish hairy root lines.

In embodiments, root tips were can then be inoculated into flasks containing growth medium (see, e.g., J. Condori et al., Plant Physiology and Biochemistry, 48 (2010) 310-318). The growth medium can be the MSV medium which is a modified MS medium as follows: Murashige and Skoog's (MS) medium [Murashige and Skookg, 1962] modified to contain 7.7 mM NH4 (from NH4NO3) and 22.85 mM NO3 (from NH4NO3 and KNO3). See, for example, T. Murashige, F. Skoog, A revised medium for rapid growth and bioassays with tobacco tissue cultures. Physiol. Plant. 15 (1962) 473e497.

In embodiments, elicitors can be added to the growth medium and incubated for 192 hours. In embodiments, the elicitors comprise B-cyclodextrin, magnesium chloride, and hydrogen peroxide. In further embodiments, the roots and media can be collected and extracted with solvent.

The term “extracting” can refer to separating into another phase. For example, extracting can refer to separating components from a solid material, e.g., provided solid plant material, into a liquid phase. Alternatively, extracting can refer to separating components from a solid material into a vapor phase, and optionally condensing said vapors. In some embodiments, extracting can refer to extracting components in an aqueous phase into an organic phase.

As used herein the term “contacting a solid material with and extractant” can refer to any form of contacting, e.g., washing the solid with the extractant, mixing the two or driving the extractant through a column containing the solid to produce a liquid extractant.

In embodiments, the extracting step can be performed at about 20° C. to about 37° C. to form a liquid extract. For example, the mixture can be agitated for a period of time (such as 24 hours at room temperature (˜25-30° C.), followed by centrifugation at 4000×g for 10 min to remove solid plant material. The supernatant (extract) was then filtered through a 0.2 uM filter to sterilize. The skilled artisan will recognize that any sterilization technique can be used to sterilize the extract, such as filtration, high heat (e.g., autoclave), or UV irradiation.

Once the solid plant material has been contacted with (i.e., combined with) the extractant, the liquid phase (i.e., liquid extract) can be separated from the remaining solid phase (i.e., plant material), thereby producing a plant extract.

In embodiments, the methods herein comprise administering to a subject a therapeutic botanical composition as described herein. The term “subject” or “patient” can refer to any organism to which aspects of the invention can be administered, e.g., for experimental, diagnostic, prophylactic, cosmetic and/or therapeutic purposes. For example, subjects to which botanical compositions can be administered include animals, such as mammals. Non-limiting examples of mammals include primates, such as humans. A wide variety of subjects are suitable for veterinary applications, such as livestock such as cattle, sheep, goats, cows, swine, and the like; poultry such as chickens, ducks, geese, turkeys, and the like; and domesticated animals for example pets such as dogs and cats. A wide variety of mammals are suitable subjects for diagnostic or research applications, such as rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

As used herein, “treatment” and “treating” can refer to the management and care of a subject for the purpose of combating a condition, disease or disorder, such as skin pigmentation disorder in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

The phrase “alleviating a symptom of” can refer to ameliorating, reducing, or eliminating any condition or symptom associated with skin pigmentation disorders. As used herein, the term “skin pigmentation disorder” can refer to disorders which affect the color of the skin. For example, skin pigmentation disorders can comprise age spots, sun damage, acanthosis nigricans, lentigo, incontinentia pigmenti, melasma, progressive pigmentary purpura, Addison's disease, skin manifestations of liver disease or liver failure, solar ephelides, and hyperpigmentation.

Aspects of the invention are further drawn to compositions and methods of treating, ameliorating, or preventing disease or disorder associated complications. For example, a disease or disorder associated complication can adversely affect the prognosis, or outcome, of a disease or disorder.

An aspect of the invention is also directed to methods of preventing or delaying the onset of one or more symptoms of a skin pigmentation disorder.

Aspects of the invention are also drawn to methods of ameliorating a symptom of a skin pigmentation disorder. “Ameliorating a symptom” can refer to an improvement of at least one discernible symptom or at least one measurable physical parameter of a disease or disorder. For example, symptoms of a skin pigmentation disorder can comprise hyperpigmentation, color changes in skin, and hypopigmentation. In embodiments, hyperpigmentation can comprise melasma, solar lentigines and ephelides, for example.

Embodiments described herein comprise administering to a subject a therapeutically effective amount of a botanical composition described herein. The term “therapeutically effective amount” as used herein can refer to that amount of an embodiment of the botanical composition or plant extract therein administered that will relieve to some extent one or more of the symptoms of the skin pigmentation disorder being treated, and/or that amount that will prevent, to some extent, one or more of the symptoms of the skin pigmentation disorder. In one embodiment, the “effective amount” can refer to the amount of an agent that modulates skin pigmentation or skin color. As used herein, the term “modulate” can refer to whitening, lightening, brightening, or increasing the radiance of at least a portion of the skin of a subject. As used herein, the term “modulate” can refer to decreasing the melanin content of skin or hair.

The term “administering” or “administration” can refer to introducing a botanical composition described herein into a subject. Any route of administration can be utilized in embodiments herein, including but not limited to intranasal, topical, oral, intranasal, parenteral, intravitreal, intraocular, ocular, subretinal, intrathecal, intravenous, subcutaneous, transcutaneous, intracutaneous, intracranial, intravenous, peritoneal, intra-arterial, inhalation, vaginal, rectal, introduction into the cerebrospinal fluid, intravascular (such as in veins or arteries), or instillation into body compartments. In embodiments, “administering” can also refer to providing a therapeutically effective amount of a botanical composition to a subject.

A therapeutic botanical composition can be formulated to be compatible with its intended route of administration, such as those described herein. In any case, the therapeutic botanical composition to be administered can contain a quantity (e.g., therapeutically effective amount) of the botanical composition necessary to treat the subject.

The therapeutic botanical composition can be administered alone, but can also be administered with other compounds, such as excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; tinctures, lozenges, capsules; tablets; granules; pellets; powders; suspensions, emulsions, or microemulsions; patches; micelles; liposomes; vesicles; implants, including microimplants; eye drops; other proteins and peptides; synthetic polymers; microspheres; or nanoparticles.

Oral compositions can include an inert diluent or an edible carrier. They can be enclosed in gelatin capsules or compressed into tablets. For the purpose of oral therapeutic administration, the active compound can be incorporated with excipients and used in the form of tablets, lozenges, troches, or capsules. Oral compositions can also be prepared using a fluid carrier, such as for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In embodiments, the therapeutic botanical composition can be administered to a subject prior to exposure to the sun, after exposure to the sun, or after appearance of age spots, skin pigmen or color changes. For example, the botanical composition can be administered to a subject after exposure to the sun as treatment for hyperpigmentation.

Multiple doses of the pharmaceutical composition can be administered to the subject. The frequency of administration of the pharmaceutical composition can vary depending on any of a variety of factors, e.g., severity of the symptoms and/or characteristics of the subject being treated. For example, in an embodiment, the pharmaceutical composition can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), four times a day, weekly, monthly, quarterly, semi-annually, annually, or by another regulator interval.

Aspects of the invention are drawn towards a method of decreasing the melanin content of human skin or hair. “Melanin” can refer to molecules responsible for pigmentation of skin and hair. For example, melanin can be produced by melanocytes

Aspects of the invention are also drawn towards a method of modulating skin pigmentation or skin color in a subject. “Skin pigmentation” can refer to the color of skin due to the amount of melanin.

In embodiments, the subject can be afflicted with a skin pigmentation disorder.

In embodiments, the subject can refer to an organism which can be administered a cosmetic composition as described herein.

### Kits

A “kit” or “medical kit” can comprise a dosage form of a botanical composition described herein. A kit can include the botanical composition along with other active ingredients, such as in combination (e.g., in a single extract), or provided separately, such as in two extracts.

Kits can further comprise one or more additional active agents. For example, the additional active agent can comprise a botanical composition, an anti-aging agent, an anti-oxidant, an anti-inflammatory agent, and the like. Kits can further comprise devices that are used to administer the active ingredients. Kits can also comprise printed instructions for administering the compound to a subject.

Kits can further comprise pharmaceutically acceptable excipients that can be used to administer one or more active ingredients. For example, if an active ingredient is provided in a solid form that must be reconstituted prior to administration, the kit can comprise a sealed container of a suitable vehicle in which the active ingredient can be dissolved to form a particulate-free sterile solution that is suitable for administration.

### Examples

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Hairy Root Establishment and Normal Root Elicitation

Establishment of Hair Roots (Source-Spain)
Germination of seeds of Morus alba (Source: FMB; Medina-Bolivar garden)
Normal root cultures of Morus alba (Source: Ukraine)
Ukraine White Mulberry (Morus alba) Normal Root

### Previous Experiment:

1. Ukraine White Mulberry normal root were excised into ½ MSMN medium with 1 mg/L NAA from 4-month-old in-vitro seedlings.
2. The normal root was cultured for 25 days at room temperature 23-24° C., then elicited with 125 μM MeJA, 18 g/L CD, 3 mM H₂O₂, and 1 mM MgCl₂.
3. Medium: 5 mL Medium of 48, 96, 144 and 192 hours was collected, and extracted by 5 mL ethyl acetate. The organic phase was dried under N₂ gas and resuspended with 0.5 mL methanol for HPLC.
4. Roots: Roots at 48, 96, 144 and 192 hours upon elicitation was collected and lyophilized. 40 mg of lyophilized root ground, by adding 2 mL of 95% ethanol (v: v), sonicated for 10 min. After centrifuge, the supernatant was used for HPLC injection.

(48, 96, and 144 hours groups have three replicates, but 192 hours group only have one.)

### New Experiment:

6 flasks roots grown at room temperature in flasks for 25 days
3 flasks for control.
3 flasks for elicited with CD+MgCl₂+H₂O₂.

Time course for medium at 48, 9, 144, 192 hours upon treatment. Root only collected at end time point 192 hours.

### Non-Limiting, Exemplary Results:

1. Oxyresveratrol is the only compound found in CONTROL MEDIUM.
2. CD+MgCl₂+H₂O₂ vs. CD MgCl₂+MeJA+H₂O₂:
Oxyresveratrol (13), Resveratrol (15), Prenyl-Resveratrol (not found in CD+MgCl₂+MeJA+H₂O₂ group). (Prenyl-Oxyresveratrol? ↑10). Moracin M has similar yield. Moracin C (↓1.7).
3. Other than the known compounds, CD+MgCl₂+H₂O₂ induced a lot more uncharacterized compounds than CD+MgCl₂+MeJA+H₂O₂ after 192 hours treatment.

### Mass Spectrometry

### Example 2—Non-Limiting, Exemplary Liquid Chromatography-Mass Spectrometry (LC-MS) Procedure for the Identification of Stilbenoids, Aryl Benzofurans and Flavone Derivatives in Normal Root and Hairy Root Cultures of White Mulberry (M. alba)

The UltiMate 3000 ultra-high-performance liquid chromatography (UHPLC) system (Dionex, Thermo Scientific) was used for chromatography. Chromatographic separations of was carried out on a reverse phase C18 ACQUITY UPLC HSS T3 (2.1×100 mm, particle size 1.8 μm, Waters) column using 0.5% aqueous formic acid (A) and ACN (B) as the mobile phases at a flow rate of 0.5 mL/min. The linear gradient started with 15% (B), 4 min 50% (B), 14 min 65% (B), 16 min 100% (B), 20 min 15% (B). The injection volume was 5 μl. UV chromatograms were recorded at 265 and 320 nm. Mass spectrometry was performed on a LTQ XL linear ion trap (Thermo Scientific) with an electrospray ionization (ESI) source. Ultrahigh pure helium (He) was used as the collision gas and high purity nitrogen (N2) as the sheath and auxiliary gas. All mass spectra were performed in both positive and negative ion mode with ion spray voltage at 4 kV, sheath gas at 45 arbitrary units (AU), auxiliary gas at 15 AU, and capillary temperature at 300° C. Full mass scans were recorded in the range m/z 100-2000. Collision-induced dissociation (CID) was used for breakage of the molecular ion into smaller fragments. The relative collision energy was set at 35% of the maximum to produce optimum yields of fragment ions. The data were analyzed using the Xcalibur software (Thermo Scientific). Compounds were tentatively identified by comparison of mass data with Dictionary of Natural Products (www.dnp.chemnetbase.com) and Dictionary of Flavonoids. 1.2

### References Cited in this Example

Buckingham, J. Dictionary of Natural Products; Chapman & Hall: London, 1993.
Buckingham, J.; Munasinghe, V. R. N. Dictionary of Flavonoids with CD-ROM; CRC Press: Boca Raton, FL, USA, 2015

### Example 3—Table 1: Metabolites Identified in White Mulberry Normal Root and Hairy Culture of White Mulberry by LC-MS in Negative ESI Mode

| | | Accurate | Molecular | Molecular | | |
|---|---|---|---|---|---|---|
| N | RT | Mass | Ion [M − H]⁻ | formula | Putative ID | Chemical group |
| U-D2 Room temperature Hairy Root 192 h control-1 LLF11May 21 | | | | | | |
| 1 | 8.49 | 646.7249 | 645.31 | C₄₀H₃₈O₈ | Kuwanon V | Diels-Alder adduct |
| | | | | | | product |
| 2 | 13.06 | 678.7237 | 677.23 | C₄₀H₃₈O₁₀ | Kuwanon I | Diels-Alder adduct |
| | | | | | | product |
| 3 | 13.27 | 648.6977 | 647.41 | C₃₈H₃₄O₉ | Chalcomoracin | Diels-Alder adduct |
| | | | | | | product |
| 4 | 13.68 | — | 985.42 | — | — | |
| 5 | 16.75 | 662.7243 | 661.44 | C₄₀H₃₈O₉ | Kuwanon Q | Diels-Alder adduct |
| | | | | | | product |
| 6 | 17.34 | 716.8147 | 715.45 | C₄₄H₄₄O₉ | Mulberrofuran T | Diels-Alder adduct |
| | | | | | | product |
| U-D2 Room temperature Hairy Root 192 h 4 elicitor-1 LLF11 May 21 | | | | | | |
| 1 | 7.85 | 564.5813 | 563.17 | C₃₄H₂₈O₈ | Kuwanol A | Diels-Alder adduct |
| | | | | | | product |
| 2 | 8.11 | 354.3533 | 353.27 | C₂₀H₁₈O₆ | Sanggenon F | isoprene substituted |
| | | | | | | flavanone |
| | | | | | Morachalcone C | chalcone derivative |
| 3 | 8.76 | 340.3698 | 339.26 | C₂₀H₂₀O₅ | Moracin T | prenylated |
| | | | | | | arylbenzofuran |
| | | | | | | derivative |
| | | 340.3267 | | C₁₉H₁₆O₆ | Moracin U | arylbenzofuran |
| | | | | | | derivative |
| 4 | 11.07 | 650.7136 | 649.37 | C₃₉H₃₈O₉ | Kuwanol E | Diels-Alder adduct |
| | | | | | | product |
| 5 | 13.36 | 648.6977 | 647.33 | C₃₈H₃₄O₉ | Chalcomoracin | Diels-Alder adduct |
| | | | | | | product |
| 6 | 17.35 | 716.8147 | 715.45 | C₄₄H₄₄O₉ | Mulberrofuran T | Diels-Alder adduct |
| | | | | | | product |

U-D2 Room temperature Hairy Root 192 h 3MeJA-1 LLF11May 21

| | | | | | | |
|---|---|---|---|---|---|---|
| 1 | 7.81 | 564.5813 | 563.31 | C₃₄H₂₈O₈ | Kuwanol A | Diels-Alder adduct |
| | | | | | | product |
| 2 | 8.11 | 354.3533 | 353.27 | C₂₀H₁₈O₆ | Sanggenon F | isoprene substituted |
| | | | | | | flavanone |
| 2 | 8.11 | | | C₂₀H₁₈O₆ | Morachalcone C | chalcone derivative |
| 3 | 8.81 | 340.3698 | 339.26 | C₂₀H₂₀O₅ | Moracin T | prenylated |
| | | | | | | arylbenzofuran |
| | | | | | | derivative |
| | | 340.3267 | | C₁₉H₁₆O₆ | Moracin U | arylbenzofuran |
| | | | | | | derivative |
| 4 | 11.07 | 650.7136 | 649.36 | C₃₉H₃₈O₉ | Kuwanol E | Diels-Alder adduct |
| | | | | | | product |
| 5 | 13.33 | 648.6977 | 647.41 | C₃₈H₃₄O₉ | Chalcomoracin | Diels-Alder adduct |
| | | | | | | product |
| 6 | 17.36 | 716.8147 | 715.47 | C₄₄H₄₄O₉ | Mulberrofuran T | Diels-Alder adduct |
| | | | | | | product |

U-D2 Room temperature Hairy Root 192 h 3H2O2-1 LLF11May21

| | | | | | | |
|---|---|---|---|---|---|---|
| 1 | 7.82 | 564.5813 | 563.26 | C₃₄H₂₈O₈ | Kuwanol A | Diels-Alder adduct |
| | | | | | | product |
| 2 | 8.12 | 354.3533 | 353.20 | C₂₀H₁₈O₆ | Sanggenon F | isoprene substituted |
| | | | | | | flavanone |
| | | | | C₂₀H₁₈O₆ | Morachalcone C | chalcone derivative |
| 3 | 8.82 | 340.3698 | 339.35 | C₂₀H₂₀O₅ | Moracin T | prenylated |
| | | | | | | arylbenzofuran |
| | | | | | | derivative |
| | | 340.3267 | | C₁₉H₁₆O₆ | Moracin U | arylbenzofuran |
| | | | | | | derivative |
| 4 | 11.09 | 650.7136 | 649.31 | C₃₉H₃₈O₉ | Kuwanol E | Diels-Alder adduct |
| | | | | | | product |
| 5 | 13.34 | 648.6977 | 647.28 | C₃₈H₃₄O₉ | Chalcomoracin | Diels-Alder adduct |
| | | | | | | product |
| 6 | 17.37 | 716.8147 | 715.41 | C₄₄H₄₄O₉ | Mulberrofuran T | Diels-Alder adduct |
| | | | | | | product |

Mulberry normal root room temperature elicitation 192 h medium 20 Mar 21

| 1 | 6.07 | 272.2527 | 271.12 | C₁₅H₁₂O₅ | 2′,4′,7′- | Flavonone |
|---|---|---|---|---|---|---|
| | | | | | Trihydroxyflavanone | |
| 2 | 7.81 | | 563.60 | — | — | |
| 3 | 8.47 | 646.7249 | 645.41 | C₄₀H₃₈O₈ | Australisin B | Diels-Alder adduct |
| | | | | | | product |
| 4 | 8.56 | — | 645.43 | — | — | |
| 5 | 11.06 | 650.7136 | 649.50 | C₃₉H₃₈O₉ | Kuwanol E | Diels-Alder adduct |
| | | | | | | product |
| 6 | 13.11 | 678.7237 | 677.47 | C₄₀H₃₈O₁₀ | Wittiorumin E | Diels-Alder adduct |
| | | | | | | product |
| 7 | 13.44 | 648.6977 | 647.31 | C₃₈H₃₄O₉ | Chalcomoracin | Diels-Alder adduct |
| | | | | | | product |
| 8 | 17.37 | 716.8147 | 715.41 | C₄₄H₄₄O₉ | Mulberrofuran T | Diels-Alder adduct |
| | | | | | | product |
| Mulberry normal root room temperature elicitation 192-1 LLF16 Mar 21 | | | | | | |
| 1 | 7.83 | — | 563.20 | — | — | |
| 2 | 8.50 | 648.6977 | 647.45 | C₃₉H₃₆O₉ | Wittiorumin F | Diels-Alder adduct |
| | | | | | | product |
| 3 | 8.59 | 692.7072 | 691.39 | C₄₀H₃₆O₁₁ | Wittiorumin C | Diels-Alder adduct |
| | | | | | | product |
| 4 | 13.44 | 648.6977 | 647.31 | C₃₈H₃₄O₉ | Chalcomoracin | Diels-Alder adduct |
| | | | | | | product |
| 5 | 13.85 | 420.4544 | 419.28 | C₂₅H₂₄O₆ | Sanggenol O | Isoprenylated flavonoid |
| 6 | 17.37 | 716.8147 | 715.41 | C₄₄H₄₄O₉ | Mulberrofuran T | Diels-Alder adduct |
| | | | | | | product |
| Mulberry normal root room temperature elicitation 48-1 medium 20 Mar 21 | | | | | | |
| 1 | 5.60 | 242.2268 | 241.11 | C₁₄H₁₀O₄ | Moracin M | arylbenzofuran |
| | | | | | | derivative |
| 2 | 8.52 | — | 645.42 | — | — | |
| 3 | 8.59 | — | 647.45 | — | — | |
| 4 | 8.82 | 340.3698 | 339.33 | C₂₀H₂₀O₅ | Moracin T | prenylated |
| | | | | | | arylbenzofuran |
| | | | | | | derivative |
| | | 340.3267 | | C₁₉H₁₆O₆ | Moracin U | arylbenzofuran |
| | | | | | | derivative |
| 5 | 9.84 | 662.7243 | 661.38 | C₄₀H₃₈O₉ | Kuwanon Q | Diels-Alder adduct |
| | | | | | | product |
| 6 | 11.39 | — | 659.28 | — | — | |
| 7 | 13.36 | 648.6977 | 647.38 | C₃₈H₃₄O₉ | Chalcomoracin | Diels-Alder adduct |
| | | | | | | product |
| 8 | 17.38 | 716.8147 | 715.48 | C₄₄H₄₄O₉ | Mulberrofuran T | Diels-Alder adduct |
| | | | | | | product |
| U-D2 24 C-room CD + Mg + H2O2 192-1 LLF01 May 21 hairy root (medium) | | | | | | |
| 1 | 4.54 | 244.2426 | 243.19 | C₁₄H₁₂O₄ | Oxyresveratrol | stilbenoid |
| 2 | 5.60 | 242.2268 | 241.11 | C₁₄H₁₀O₄ | Moracin M | arylbenzofuran |
| | | | | | | derivative |
| 3 | 8.04 | 634.62 | 633.30 | C₃₁H₃₈O₁₄ | Moracin C 3′,5′- | arylbenzofuran |
| | | | | | diglucoside | derivative |
| 4 | 8.84 | 340.3698 | 339.30 | C₂₀H₂₀O₅ | Moracin T | prenylated |
| | | | | | | arylbenzofuran |
| | | | | | | derivative |
| | | 340.3267 | | C₁₉H₁₆O₆ | Moracin U | arylbenzofuran |
| | | | | | | derivative |
| 5 | 12.17 | — | 987.37 | — | — | |
| 6 | 13.39 | 648.6977 | 647.37 | C₃₈H₃₄O₉ | Chalcomoracin | Diels-Alder adduct |
| | | | | | | product |
| 7 | 17.40 | 716.8147 | 715.26 | C₄₄H₄₄O₉ | Mulberrofuran T | Diels-Alder adduct |
| | | | | | | product |

U-D2 24C-room CD + Mg + H2O2 48-1 LLF01 May 21 hairy root (medium)

| | | | | | | |
|---|---|---|---|---|---|---|
| 1 | 4.57 | 244.2426 | 243.19 | C₁₄H₁₂O₄ | oxyresveratrol | Stilbenoid |
| 2 | 8.02 | 634.62 | 633.29 | C₃₁H₃₈O₁₄ | Moracin C 3′,5′- | arylbenzofuran |
| | | | | | diglucoside | derivative |
| 3 | 8.80 | 340.3698 | 339.30 | C₂₀H₂₀O₅ | Moracin T | prenylated |
| | | | | | | arylbenzofuran |
| | | | | | | derivative |
| | | 340.3267 | | C₁₉H₁₆O₆ | Moracin U | arylbenzofuran |
| | | | | | | derivative |
| 4 | 12.09 | — | 987.43 | — | — | |
| 5 | 13.27 | 648.6977 | 647.41 | C₃₈H₃₄O₉ | Chalcomoracin | Diels-Alder adduct |
| | | | | | | product |
| 6 | 17.34 | 716.8147 | 715.45 | C₄₄H₄₄O₉ | Mulberrofuran T | Diels-Alder adduct |
| | | | | | | product |

### Example 4—Elicitation of Stilbenes and Benzofuran Derivatives in Hairy Root Cultures of White Mulberry (Morus alba)

White mulberry (Morus alba) is a traditional Chinese medicinal plant rich in bioactive compounds. Stilbene and benzofuran derivatives isolated from the root have shown various biological activities, including anti-inflammatory, antioxidant, and antimicrobial properties. To develop a sustainable system for producing these compounds, hairy root cultures of white mulberry were established, and the effect of different elicitor combinations on the production of stilbenes and benzofurans was studied. Upon co-treatment with 18 g/L cyclodextrin, 3 mM hydrogen peroxide and supplementation with 1 mM magnesium chloride several stilbenes and aryl benzofurans, including non-prenylated and prenylated derivatives, were induced and accumulated in the culture medium. The stilbenes oxyresveratrol, resveratrol, and 3′-prenylresveratrol accumulated in the culture medium up to 6.27 mg/g DW, 0.61 mg/g DW, and 5.00 mg/g DW, respectively. Whereas the oxyresveratrol diglucoside mulberrroside was only detected in the root tissue with yields up to 10.01 mg/g/DW. At the same time, the levels of the aryl benzofuran moracin M and its prenylated derivative moracin C accumulated in the culture medium up to 7.82 mg/g DW and 1.82 mg/g DW, respectively. Chalcomoracin was also induced and accumulated in the medium upon elicitation. The results demonstrated that the integration of multiple elicitors can significantly enhance stilbene and benzofuran production in M. alba hairy root cultures.

White mulberry (Morus alba L.) is a deciduous tree native to China widely disseminated throughout Asia, Africa, Europe, and South and North America [1,2]. Leaves, root bark, stem, and fruits of M. alba have been used in traditional Chinese medicine for the treatment of metabolic disorders such as diabetes, hyperlipidemia, and high blood pressure [3-6]. Chemical investigation of different tissues from white mulberry revealed that phenolic compounds, such as flavonoids, stilbenes, benzofurans, and Diels-Alder type adducts are the main constituents [7].

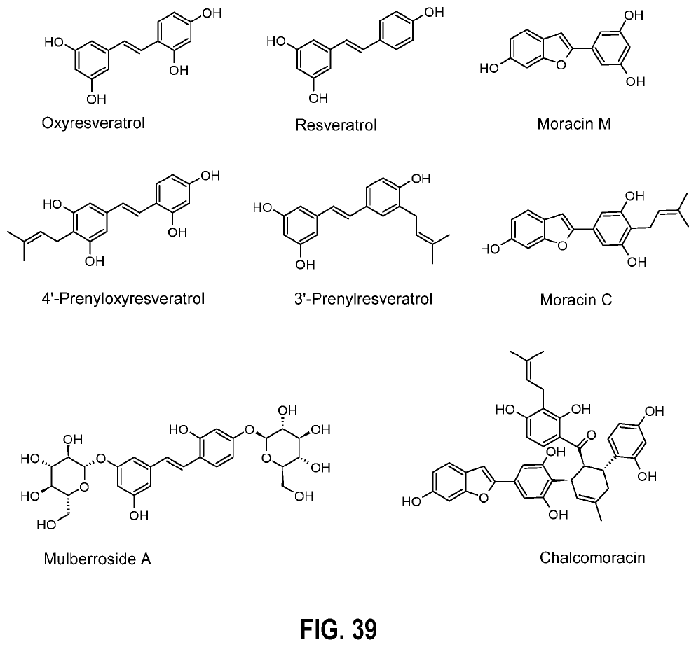

Stilbenes are polyphenolic phytoalexins produced by certain plants in response to biotic or abiotic stress. These compounds have remarkable biological properties [8]. Among these, resveratrol (trans-3,4′,5-trihydroxystilbene; FIG. 39), has a simple chemical structure and, without wishing to be bound by theory, can be used in therapeutic applications in various diseases [9]. Oxyresveratrol (trans-2,4,3′,5′-tetrahydroxy stilbene; FIG. 39) another natural hydroxystilbene, exhibits more powerful antioxidant activity when compared to resveratrol due to the presence of an additional hydroxyl group [10]. Furthermore, it has anti-inflammatory [11,12], antiviral [13], and neuroprotective activities. Oxyresveratrol, along with its diglucoside mulberroside A (FIG. 39), has anti-browning [15], and tyrosinase inhibition [16-18], and therefore can be used as raw materials for skin-whitening cosmeceuticals [19].

Among the benzofurans, moracin M can be isolated from the root bark of M. alba L. and has anti-inflammatory activity [20,21]. Moracin C is a natural 2-arylbenzofuran prenylated derivatives isolated from fungus-infected mulberry leaves [22]. It is in Artocarpus heterophyllus (commonly known as jackfruit tree) [23]. Moracin C has antibacterial [24], anticancer [25,26], antioxidant [27], α-glucosidase [28], and lipoxygenase inhibitory activities

Chalcomoracin is a Diel-Alder type adduct produced by fungus-infected mulberry leaves [29]. It exhibits biological activities against methicillin-resistant Staphylococcus aureus (MRSA) [30,31], and human cancer cell lines [32,33]. It is formed through the Diels-Alder type cycloaddition of a prenylchalcone and a prenylated 2-arylbenzofuran [29].

Hairy root cultures produced via Agrobacterium rhizogenes-mediated transformation are a sustainable platform for the production of valuable specialized metabolites [34,35]. Chemical elicitation of hairy roots can be used to increase the yield of metabolites. The combination of different elicitor combinations including methyl jasmonate (MeJA), methyl-β-cyclodextrin (CD), hydrogen peroxide (H₂O₂), and supplementation with magnesium chloride (MgCl₂) leads to the elicitation and secretion of high levels of the prenylated stilbenes arachidin-1 and arachidin-3 and the prenylated stilbene derivative cajaninstilbene acid into the medium of hairy root cultures of peanut and pigeon pea, respectively. Though, these multiple-elicitor treatments have not been studied in hairy root cultures white mulberry.

In this paper, we report the establishment of hairy root cultures of white mulberry (M. alba) and describe three strategies to elicit the production of stilbenes and aryl benzofurans including their prenylated derivatives. The elicitation strategies included (A) MeJA, H₂O₂, CD, and MgCl₂, (B) H₂O₂, CD and MgCl₂ and (C) MeJA, CD, and MgCl₂. Time course studies revealed that different combinations of elicitors have a distinct effect on the levels of selected stilbenes and aryl benzofurans.

2.1 Development and Characterization of Hairy Root Cultures of M. alba

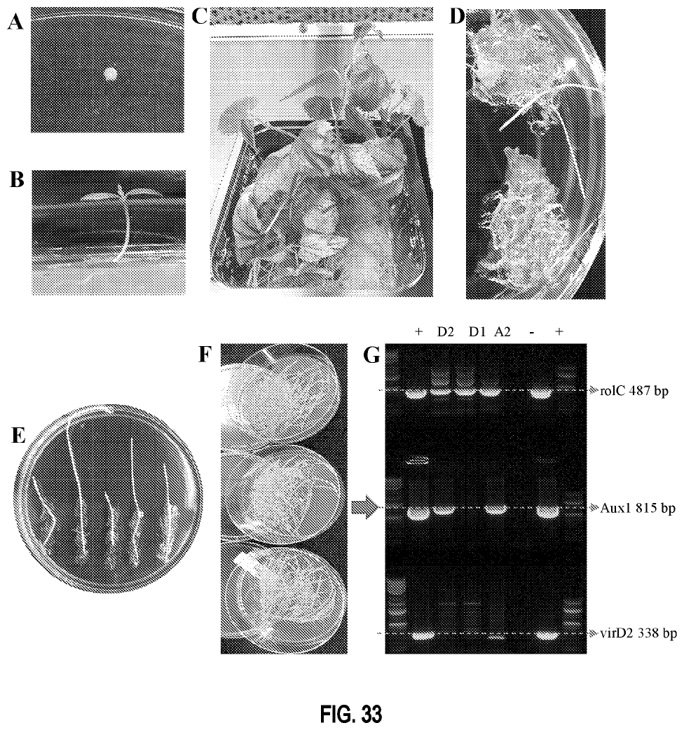

Surface-sterilized seeds of white mulberry were germinated in vitro to provide aseptic plant materials for hairy root induction. The leaves of 8-week-old seedlings were excised and wounded with A. rhizogenes ATCC 15834 (FIG. 33). After 4 to 6 weeks of inoculation, roots that developed from wounded leaves were transferred to cefotaxime-containing media to avoid agrobacteria overgrowth. After the elimination of bacteria from the hairy roots by subcultures on cefotaxime-containing media, three hairy root lines (U-A2, U-D1 and U-D2) were selected for further study due to their fast growth. The hairy roots were transferred to liquid cultures and their DNA was extracted for PCR analysis. Since A. rhizogenes ATCC 15834 contains two T-DNAs, TL-DNA (harboring rol genes) and TR-DNA (harboring aux1 and aux2 genes), the integration of the TL-DNA and TR-DNA into the host genome of the putative hairy roots was tested by screening for rol C and aux1 genes. The PCR analysis revealed the integration of the rol C gene in all transformed hairy root lines. The aux1 gene was detected in the U-D2 and U-A2 lines, but not the U-DI line. Expression of the rol genes localized in TL-DNA is necessary for the hairy root phenotype [37]. Consequently, transfer of only TL-DNA is frequent in hairy roots induced by agropine strains of A. rhizogenes such as ATCC 15834 [38]. Similarly, in our previous study with Arachis ipaensis and A. duranensis hairy roots, some lines only included the rol genes from TL-DNA [36]. The lack of amplification of the virD2 gene (localized outside the T-DNAs of the root-inducing plasmid) in the white mulberry hairy root lines confirmed the absence of any remaining Agrobacterium in the root tissue (FIG. 33, panel G). Among the three established lines, hairy root line U-D2 harboring both rol and aux genes was selected for further analysis due to its vigor and sustained growth in liquid culture.

2.2 Elicitation of Stilbenes and Moracins in Hairy Root Cultures of M. alba

Elicitation is a strategy to induce and enhance the secretion of specialized metabolites in hairy root cultures. Our group optimized an elicitation procedure in peanut hairy root cultures to enhance the production of prenylated stilbenes. Under co-treatment with CD, MeJA, H₂O₂, and MgCl₂, the overall yield of prenylated stilbenoids in the medium reached approximately 750 mg/L. Recently, this multiple-elicitor procedure led to a 277-fold increase in the levels of the prenylated stilbene derivative cajaninstilbene acid in hairy root cultures of pigeon pea.

Root and stem barks of white mulberry accumulates prenylated stilbenes and 2-arylbenzofuran derivatives including prenylresveratrol, moracin M, and moracin C [39-41]. To establish a sustainable system to produce these bioactive compounds, 35-day-old hairy root cultures of white mulberry were treated with three groups of elicitors: CD+MgCl₂+H₂O₂, CD+MeJA+MgCl₂, and CD+MeJA+MgCl₂+H₂O₂. Seven major compounds, including the stilbenes oxyresveratrol, resveratrol, 3′-prenyl-resveratrol and mulberrroside A and arylbenzofurans and derivatives including moracin M, moracin C and chalcomoracin were detected. Identification was based on comparisons to the HPLC retention time, UV spectrum, and tandem mass spectrometry analyses of authentic standards. Since no standards were available for 3′-prenyl-resveratrol and chalcomoracin, their identification was based on published UV-spectra and fragmentation patterns of the mass spectrometry analyses described below.

### 2.2.1 Phenotype of Hairy Root Line U-D2 Upon Multiple Elicitors Treatments

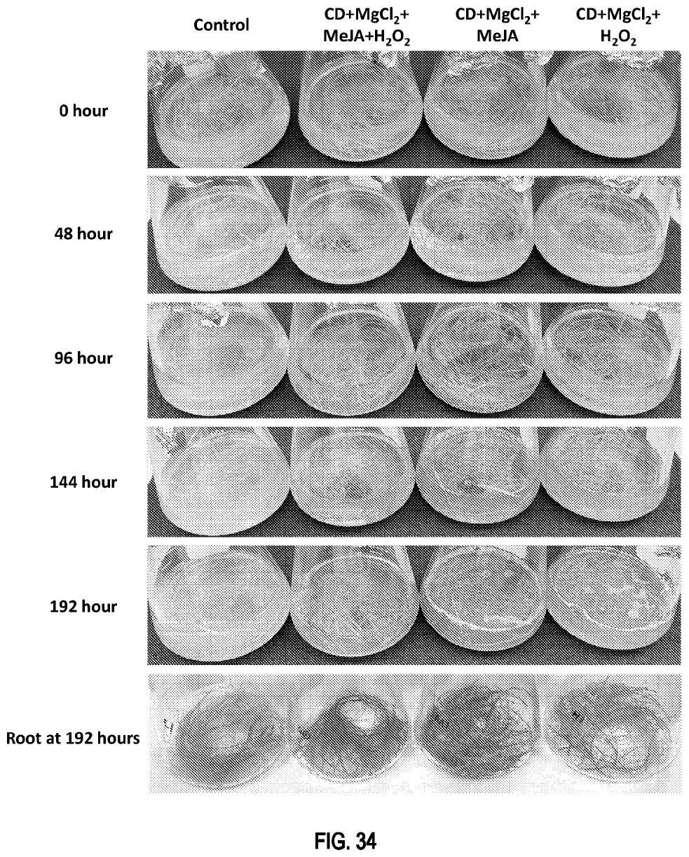

To induce and enhance the production of bioactive compounds in hairy root cultures of white mulberry, 35-day-old hairy root cultures of line U-D2 were treated with three groups of elicitors: CD+MgCl₂+H₂O₂, CD+MeJA+MgCl₂, and CD+MeJA+MgCl₂+H₂O₂. The effects of the three treatments on the production of bioactive compounds were evaluated along a time course from 48 to 192 hours. In contrast to the healthy root tissue observed in the non-treated control group, the hairy root tissue showed a brownish color after elicitor treatment with CD+MgCl₂+H₂O₂. Interestingly, the cultures treated CD+MeJA+MgCl₂, and CD+MeJA+MgCl₂+H₂O₂ showed the darkest color (FIG. 34). Upon elicitation, the color of the culture medium turned to bright light-yellow color suggesting the presence of phenolic compounds in the culture medium.

### 2.2.2 Effect of Different Elicitor Treatments on the Yield of Stilbenes and Benzofurans

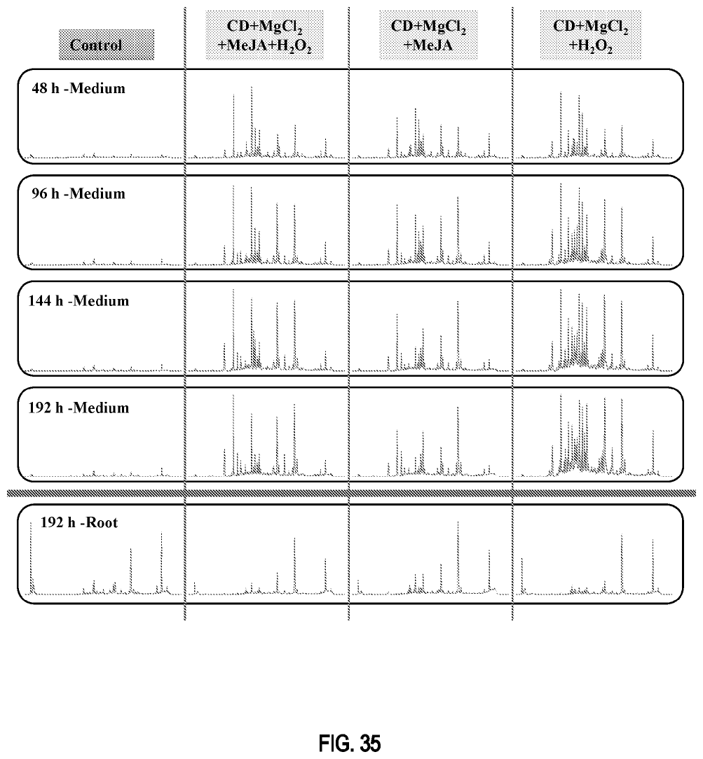

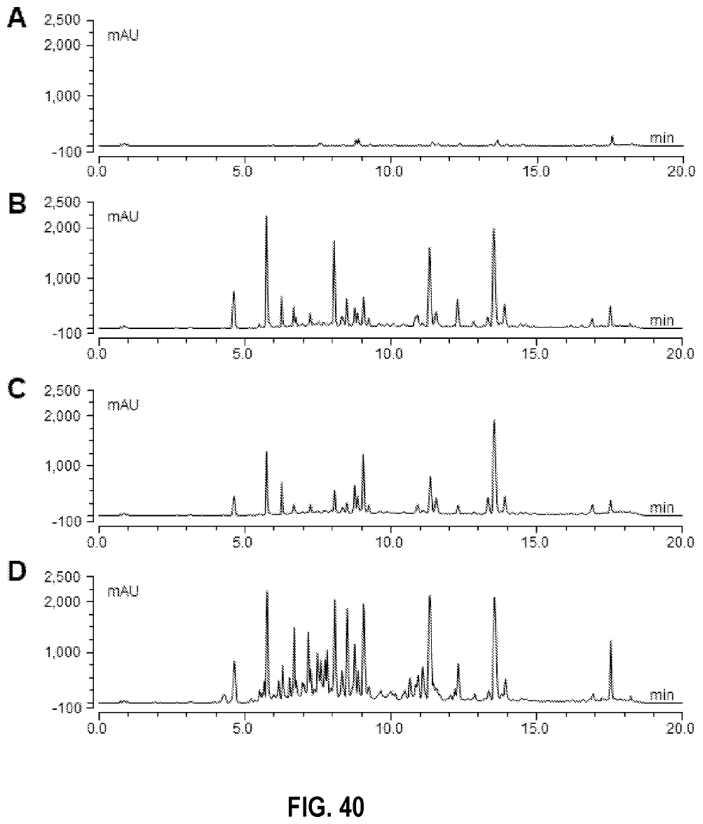

To determine the profile of phenolic compounds, white mulberry hairy root cultures of line U-D2 were elicited with CD+MgCl₂+H₂O₂, CD+MgCl₂+MeJA, and CD+MgCl₂+MeJA+H₂O₂ and ethyl acetate extracts of the culture medium after 48 to 192 hours of elicitation were analyzed by HPLC (FIG. 35 and FIG. 40). Significant amounts of phenolic compounds were found in the culture medium of the elicited hairy root cultures, whereas only very few compounds were detected in the non-treated control cultures (FIG. 40). More than half of the peaks detected by HPLC were classified as stilbenes or furans based on their specific UV-spectra with absorption maximum at 320˜330 nm and 315˜319 nm, respectively (FIG. 41). By comparing with the elution time, UV and MS/MS spectrum of authentic standards, the compounds oxyresveratrol, resveratrol, moracin M, and moracin C were identified and quantified in the extracts from the culture medium (FIG. 41).

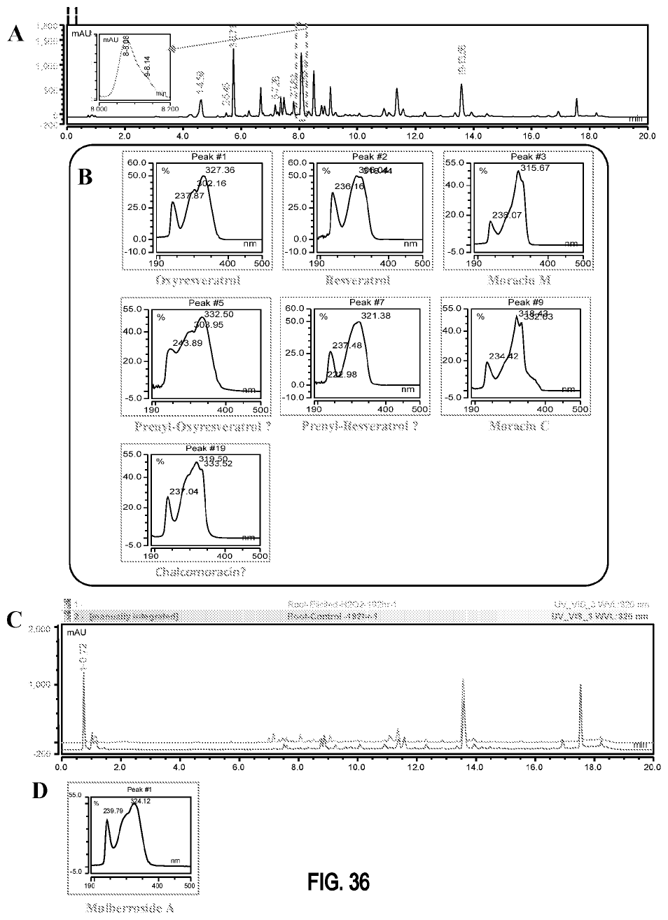

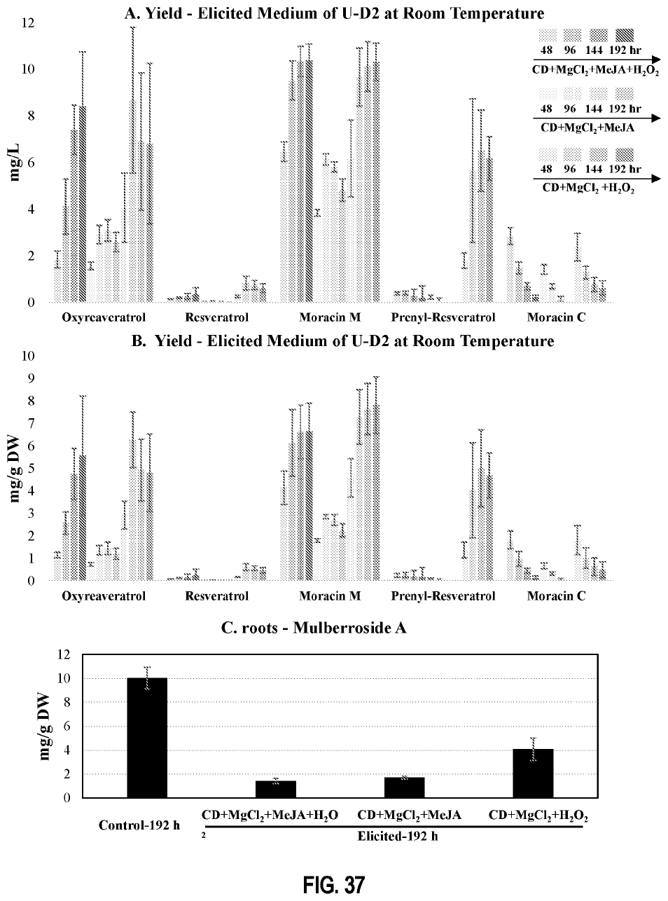

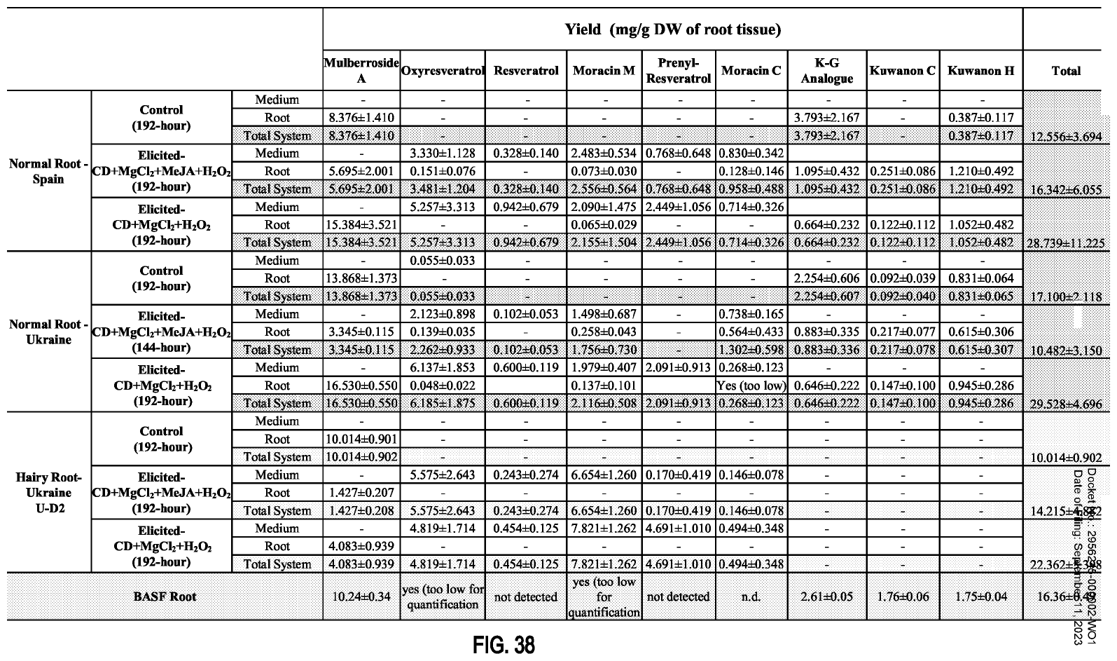

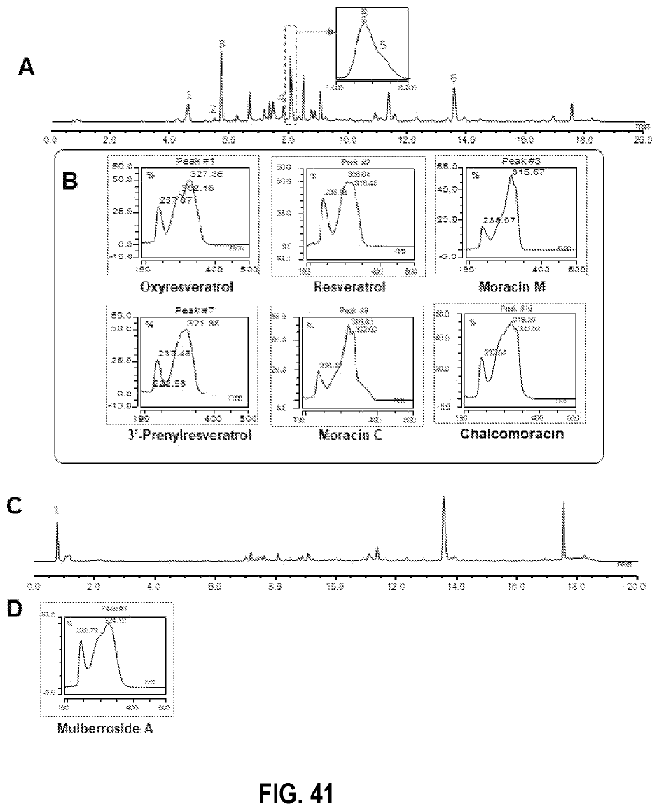

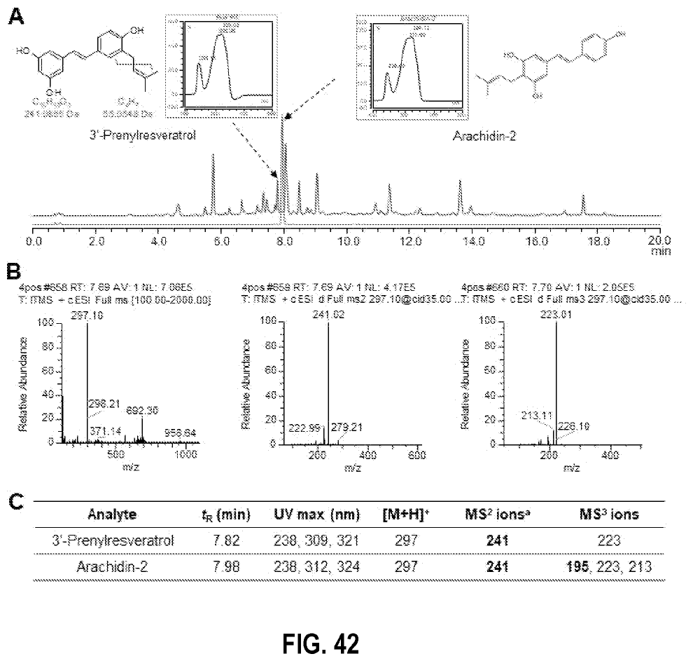

The highest yield of oxyresveratrol was 6.27±1.24 mg/g DW (equivalent to 8.67 mg/L of medium) and it was found in the medium of cultures elicited with CD+MgCl₂+H₂O₂ for 96-hours (FIG. 37). This yield was 13.93-fold higher than that 0.45±0.01 mg/g DW reported by Jeon and Choi in white mulberry leaves [39]. Upon the same elicitation treatment with CD+MgCl₂+H₂O₂, resveratrol reached its highest yield of 0.61±0.16 mg/g DW (equivalent to 0.83 mg/L of medium) in the culture medium after 96 hours treatment (FIG. 37). Besides oxyresveratrol and resveratrol, another stilbene found in elicited culture medium was identified as a prenylated resveratrol with a molecular mass of 296 m/z (or [M+1]⁺ of 297) (FIG. 42). This compound had a distinct elution time and MS/MS fragmentation pattern when compared to arachidin-2, where the prenyl group is at the C-4 position of resveratrol, indicating that the prenyl group is attached to the other aromatic ring of resveratrol.

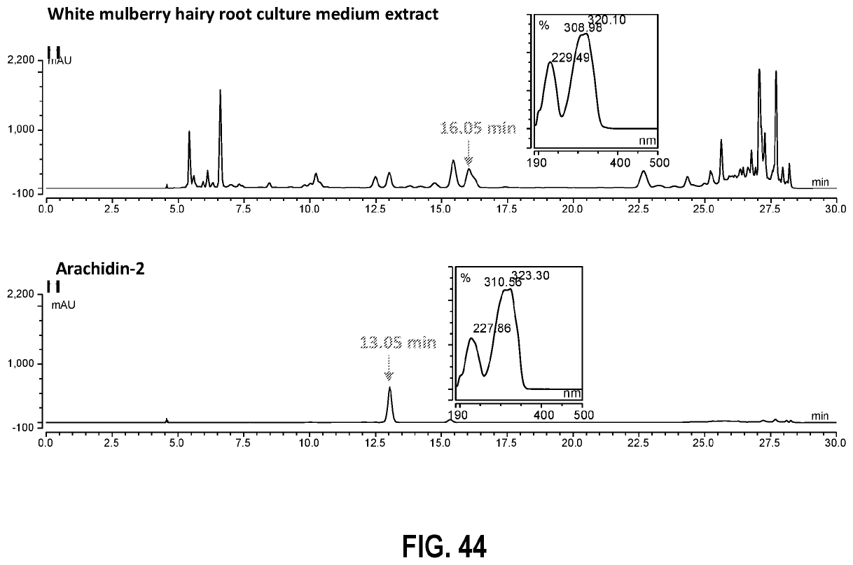

To characterize this stilbene, extracts from the culture medium of the elicited hairy root cultures were run using a different HPLC column with a protocol specific separating prenylated stilbenes (FIG. 42 & FIG. 44). We confirmed that this prenylated resveratrol had same elution time, UV and MS/MS spectrum with 3-methyl-2-butenyl-3′-resveratrol (3′-prenylresveratrol) which was the product of an enzymatic reaction of a resveratrol prenyltransferase characterized from peanut [42]. The mass spectrometry analysis of this stilbene showed a molecular ion at m/z 297 [M+H]⁺ and fragment ion at m/z 241.02 in MS², confirming the presence of a prenyl moiety. The highest yield of this 3′-prenylresveratrol was 5.00±1.71 mg/g DW (equivalent to 6.51 mg/L of culture medium) after 144-hour treatment of CD+MgCl₂+H₂O₂ in the culture medium (FIG. 37).

The aryl benzofurans moracin M and its prenylated derivative moracin C were identified and quantified in the elicited hairy root cultures of white mulberry. Moracin M accumulated to 7.82±1.26 mg/g DW (equivalent to 10.33 mg/L of culture medium) after 192-hour of elicitation with CD+MgCl₂+H₂O₂, while the yield of moracin C decreased from its highest yield of 1.82±0.65 mg/g DW (equivalent to 2.37±0.59 mg/L of culture medium) at 48 hours indicating that it is a metabolic intermediate for other furans during the elicitation periods (FIG. 37). Jeon and Choi (2019) reported that 0.17±0.0.006 mg/g DW of moracin M and 0.07±0.0.003 mg/g DW of moracin C accumulated in UV-treated leaves of white mulberry, which are about 46- and 26-fold lower than in the current hairy root culture system [39].

No dramatic changes were observed in the HPLC profiles of extracts from the hairy root tissue before and after elicitation. Mulberroside A was identified in the tissue by comparing with the retention time and UV spectrum of an authentic mulberroside A standard (FIG. 41, panels C and D). The yield of mulberroside A in the non-treated control group was 10.01 g/g DW of root, which was 2.6-fold higher than the yield in the CD+MgCl₂+H₂O₂ elicited hairy roots tissues. The latter was the highest yield among the three treatment (FIG. 37).

Unlike the above compounds having commercial standards available, a main compound found in both the culture medium and root tissue was identified as chalcomoracin by comparing its retention time, UV-spectrum, and MS/MS fragmentation patterns with those

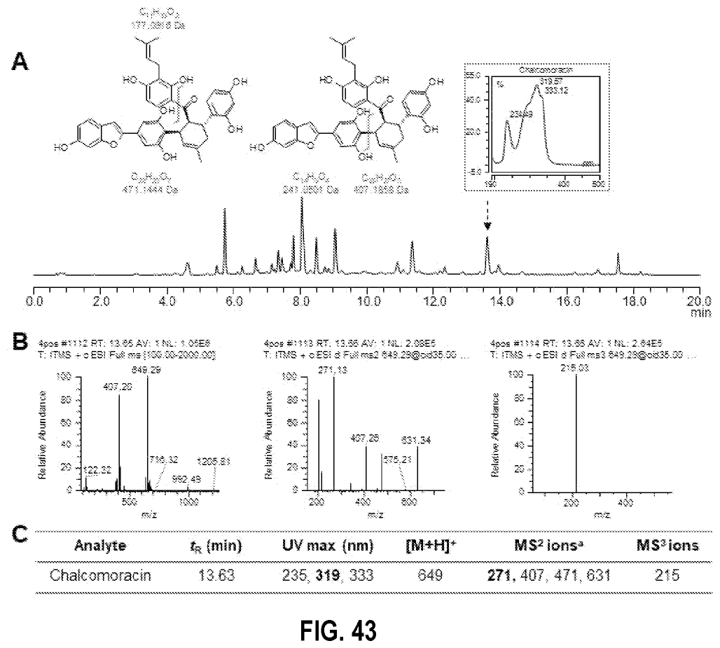

reported in the literature for chalcomoracin [39]. The UV spectrum of chalcomoracin exhibited an absorption maximum at 319-320 nm [39, 42]. The molecular ions identified in negative ESI-MS at m/z 647.34 [M−H]⁻ and positive ESI-MS at m/z 649.29 [M+H]⁺ allowed the deduction of its molecular weight at 648 Da [38, 41]. This was further corroborated by a fragmentation pattern in MS2 (FIG. 43).

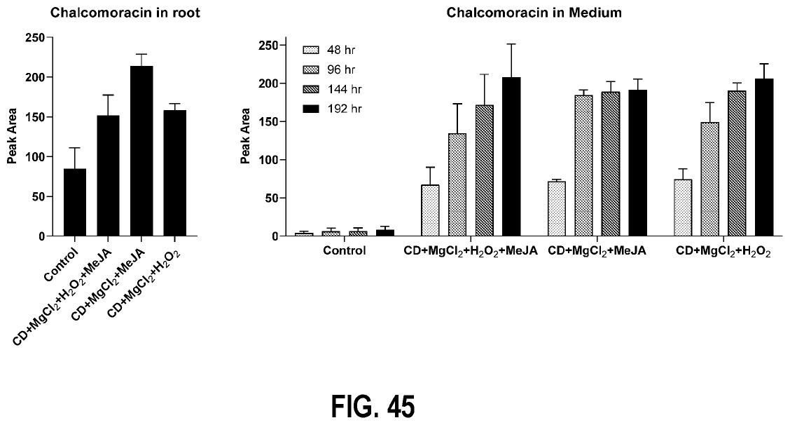

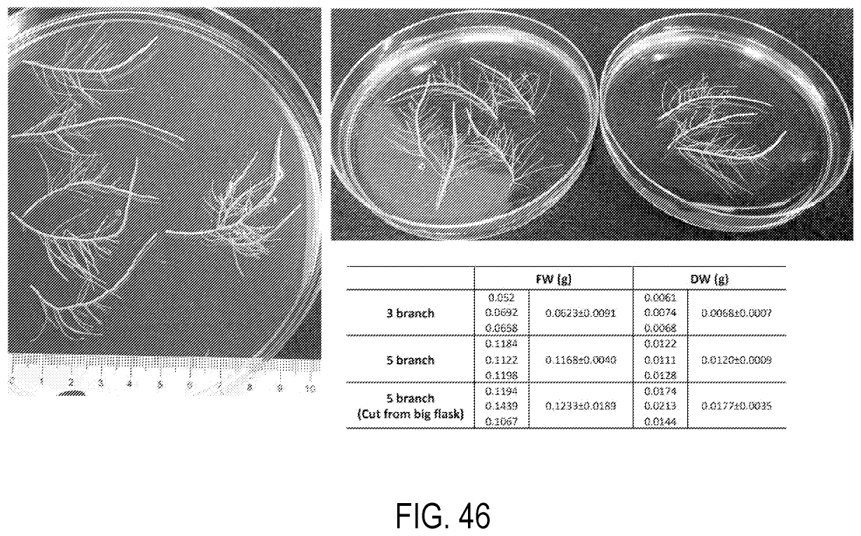

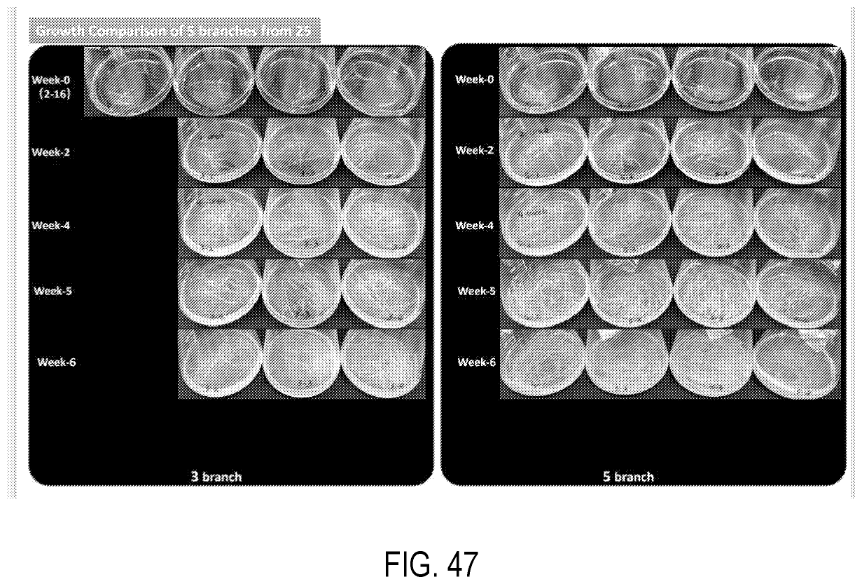

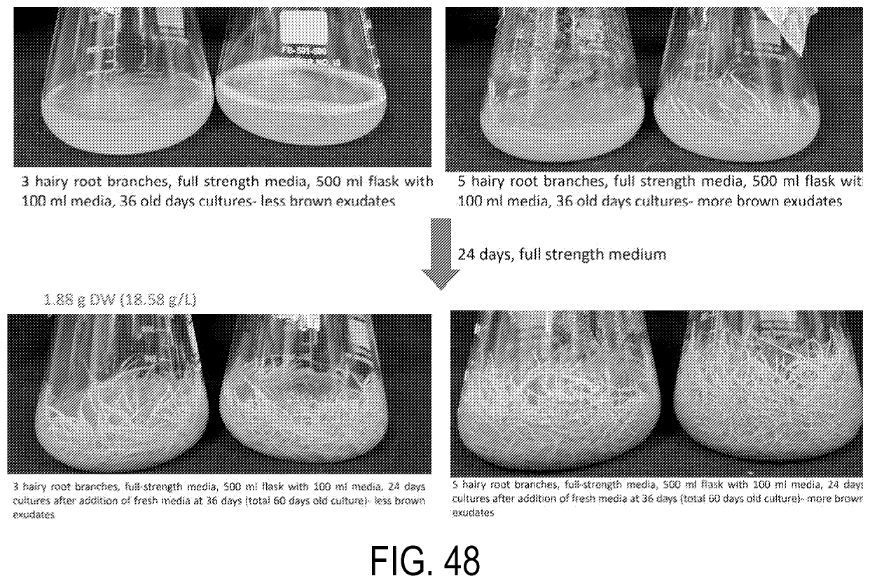

Based on the HPLC peak area, the yield of chalcomoracin in the elicited culture medium was about 23.8-fold higher than the non-elicited culture. Whereas the yield of this compound in the elicited root tissue was 1.8-fold higher when compared to the non-elicited hairy root tissue (FIG. 45).

Overall, when compared to the CD+MgCl₂+H₂O₂ treatment, the other two treatment groups CD+MgCl₂+MeJA and CD+MgCl₂+MeJA+H₂O₂ induced less accumulation of phenolic metabolites in the medium of white mulberry hairy roots. For comparison, the time course of accumulation of the identified stilbenes and aryl benozyfurans expressed in mg/g DW root and mg/L of culture medium are shown in Example 5 and Example 6 and FIG. 37. The CD+MgCl₂+MeJA+H₂O₂ treatment was initially optimized in the peanut hairy root culture for enhancing the yield of prenylated stilbenes. White mulberry hairy roots exhibit distinct and additional biosynthetic pathways (e.g., aryl benzofurans) when compared to peanut, which may explain the differences in response to the CD+MgCl₂+MeJA+H₂O₂ treatment.

### 3. Discussion

A few studies on Agrobacterium-mediated transformation of Morus have been reported [44-46]. However, none of them relates to the establishment of hairy root cultures with the capacity to produce specialized metabolites. Thus, this is the first study to report the bioproduction of stilbenes and benzofurans in hairy root cultures of white mulberry.

Chemical elicitors such as jasmonic acid, methyl jasmonate (MeJA), cyclodextrin (CD), hydrogen peroxide (H₂O₂), sodium acetate, salicylic acid, acetylsalicylic acid, ethylene, nitric oxide, sodium nitropruside, heavy metal ions, etc. have been used to include stress in plants and consequently increase the yield and accumulation of metabolites as well as produce

entirely novel/new molecules [47]. These elicitors are either used alone or in combination. In this study, we treated M. alba hairy root cultures with three different elicitors methyl jasmonate (MeJA), cyclodextrin (CD), hydrogen peroxide (H₂O₂), and supplemented the culture medium with MgCl₂ in different combinations.

Methyl jasmonate (MeJA) is a methyl ester of jasmonic acid widely distributed in the plant kingdom. MeJA is a stress-signaling molecules and is involved in the signal transduction pathway that induces particular enzymes to catalyze biochemical reactions to produce low molecular weight phytoalexins [48]. Cyclodextrins (CDs) are cyclic oligosaccharides formed by six to eight α-D-glucopyranose residues linked by α 1→4 glucosidic bonds containing a relatively hydrophobic central cavity and a hydrophilic outer surface resulting in a toroidal structure [49]. The seven-membered ring cyclodextrin is β-cyclodextrin derived from starch by the action of microbial cyclodextrin glycosyltransferases. CDs are commonly used in plant cell cultures and hairy root cultures to prevent feedback inhibition and improve the availability of hydrophobic bioactive compounds. In addition, they also act as an elicitor due to their structural resemblance to naturally released pectic oligosaccharides from the cell walls during fungal infections [49-51]. Hydrogen peroxide (H₂O₂) acts as a signaling molecule and plays an important role in the production of specialized metabolites in plants by inducing resistance to different stresses [52,53]. Indeed, previous studies showed an increased level of H₂O₂ in plants during different environmental stresses such as oxidative stress [54], salt stress [55,56], and heavy metal ions stress [57-59].

Magnesium (Mg²⁺) is the most abundant divalent cation in living cells, and is a co-factor required for the activity of prenyltransferase enzymes involved in the prenylation of phenolic compounds [60,61]. Prenylation refers to the introduction of a C5 (prenyl), C10 (geranyl), or C15 (farnesyl) isoprenoid group in a molecule and the prenyl group binds to different positions of the aromatic ring of phenolic compounds through the action of prenyltransferases [62]. Prenylation can enhance the bioactivity of phenolic compounds by increasing lipophilicity and affinity to biological membranes. Indeed, to increase the levels of prenylated derivatives of stilbenes and benzofurans, the elicitation medium was supplemented with MgCl₂.

The use of chemical elicitors to enhance the production of specialized metabolites in hairy root cultures of M. alba remains unexplored. The present study represents a quite new example of chemical elicitation to produce the biologically active secondary metabolites in hairy root cultures of this species. Specifically, the purpose of this study was to validate the influence of different combinations of elicitors and exposure times for the optimum production of stilbenes and benzofurans in hairy root cultures of M. alba.

Stilbenes are phenolic compounds produced by a limited group of plants in response to different stresses. These compounds exhibit a wealth of biological properties and potential health benefits including cardioprotective, neuroprotective, anti-inflammation, antidiabetic, and anticancer [8].

Similarly, benzofurans are biologically active heterocyclic compounds widely distributed in higher plants. Benzofurans have a variety of biological activities. Compounds containing a benzofuran skeleton have a wide range of biological activities with therapeutic values such as antibacterial, antifungal, anti-inflammatory, analgesic, anti-depressant, anticonvulsant, antitumor, anti-HIV, anti-diabetic, antituberculosis, and antioxidation [63,64].

Common metabolites of the roots of Morus species are Diels-Alder-type adducts, flavonoids, benzofurans, stilbenes, and polyhydroxylated alkaloids have a wide spectrum of biological properties [65]. Enhanced production of mulberroside A followed by oxyresveratrol and resveratrol is in root cultures of M. alba co-elicited with MeJA and yeast extract [66]. Similarly, increased production of mulberroside A can be from M. alba hairy root cultures elicited with yeast extract, MeJA, and salicylic acid [67]. In addition, three Diels-Alder type adducts, guangsangon E, chalcomoracin and sorocein I can be isolated from hairy root cultures

of Morus macroura elicited with Saccharomyces cerevisiae extract [68]. In this study, we reported the production of the stilbenes oxyresveratrol, resveratrol, 3′-prenylresveratrol, and mulberrroside A and the benzofurans moracin M, moracin C and chalcomoracin. Hairy root cultures of line UD-2 were elicited with three groups of elicitor combinations: CD+MgCl₂+H₂O₂, CD+MeJA+MgCl₂, and CD+MeJA+MgCl₂+H₂O₂. The highest levels of metabolites were found in the culture medium upon treatment with CD+MgCl₂+H₂O₂. Intriguingly, some metabolites oxyresveratrol and mulberroside A were only detected in the hairy root cultures whereas others were detected in both elicited media and hairy root. This is indicative of some metabolites being produced and accumulated intracellularly by hairy roots.

Besides the combination of elicitors and concentration, the time of elicitation is also a key factor for the synthesis of secondary metabolites. The present study revealed that 196 h elicitation resulted in the higher accumulation of selected stilbenes and benzofurans the other times tested. Interestingly, mulberroside levels were reduced upon all elicitor treatments. The latter is a glucoside of oxyresveratrol that accumulate in the tissue of the hairy roots. Elicitation can activate the hydrolysis of this glucoside leading to increase levels of oxyresveratrol.

### 4. Materials and Methods

### 4.1. Sterilization and Germination of White Mulberry Seeds

Seeds of white mulberry (M. alba var. tatarica; source country: Ukraine) were obtained from the Sheffied's seed Company https://sheffields.com/, New York, USA. Dry seeds were soaked in 50° C. warm water in a 50 mL conical tube overnight. The warm water cooled down naturally. Any remaining fruit tissue outside the seeds was removed before starting the surface sterilization. Then the seeds were dipped into 70% ethanol for 3 min, followed by 66.7% Clorox (4% V/V NaOCl) with 0.05% tween-20 for 15 min. Afterward, the seeds were put back in 70% ethanol for 1 min and rinsed thoroughly with sterilized distilled water 4-5 times. The sterilized seeds were put on plates containing MS (Murashige and Skoog; ADD REFERENCE) basal media with 30 g/L sucrose and 4 g/L phytagel and cultured at 24° C. under darkness until germination.

### 4.2. Establishment of Hairy Root Lines of White Mulberry

Fresh leaves were excised from in vitro seedlings of white mulberry established as described above and wounded with a scalpel containing Agrobacterium rhizogenes strain ATCC 15834. The wounded leaves were cultured on MSV medium for 2-3 days until bacterium growth was observed around the wounded area. Then the leaves were subcultured on MSV medium with 200 mg/L cefotaxime. Leaves were maintained in this medium until hairy roots developed. After hairy roots reached about 2 cm in length, they were excised from the leaves and placed onto a fresh MSV plate with 200 mg/L cefotaxime. Newly grown hairy root tips were cut and placed onto a MSV plate without cefotaxime. After eliminating Agrobacterium, the hairy roots were subcultured into flasks containing liquid MSV medium.

Among the several hairy root lines established, lines U-A2, U-DI and U-D2 were selected for their better growth and used for PCR analyses. The hairy roots were lyophilized in a Freeze Dry System Freezone 4.5 lyophilizer (Labconco™, Kansas City, MO, USA) and then the lyophilized hairy root tissue was used to extract genomic DNA using the DNeasy® Plant Mini kit (Qiagen, Germantown, MD, USA). PCR analyses of rolC, aux1 and virD2 genes were performed as described before [70].

### 4.3. Elicitation of Hairy Root Cultures

Thirty-five-day old hairy root cultures of line U-D2 grown in 250 mL flasks with 50 mL of MSV medium were used for elicitation. The old spent culture medium was removed and replaced by 100 mL fresh MSV medium containing three different groups of elicitors. Group-1:18 g/L methyl-β-cyclodextrin (CD; CAVASOL® W7 M, Wacker), 125 μM methyl jasmonate (MeJA; Sigma), 3 mM H₂O₂ (Fisher Scientific), and additional 1 mM MgCl₂ (Sigma), group-2:18 g/L CD, 125 μM MeJA, and additional 1 mM MgCl₂, and group-3:18 g/L CD, 3 mM H₂O₂ and additional 1 mM MgCl₂. A control group was also analyzed by only refreshing MSV medium without adding any of the elicitors mentioned above. Each group had four flasks as biological replicates.

4.4. Extraction of Phenolics from the Hairy Root Culture Medium and Tissue

A time-course elicitation experiment was conducted. Five mL aliquots of medium were collected at 48, 96, 144 and 192 hours after elicitor treatment. The aliquots were mixed with 5 mL of ethyl acetate in a 15 mL conical tube by vortexing for 30 s. After centrifugation, the upper organic phase was transferred to borosilicate glass tubes (Fisherbrand™, Fisher Scientific) and dried in a SpeedVac at 40° C. The dried residue was resuspended in 500 μL of MeOH and analyzed by reverse phase HPLC as detailed herein.

To extract the phenolic compounds from the root tissue after the elicitation treatment, the hairy roots were first rinsed with water thoroughly to get rid of any remaining medium. The hairy roots were frozen at −80° C. and then lyophilized. The dried hairy roots were ground in a mortar and with pestle. Forty mg of dried root tissue was extracted with 2 mL 95% ethanol. After centrifugation, the supernatant was analyzed by HPLC.

### 4.5. HPLC Analysis

HPLC analyses were performed in an Ultimate 3000 UHPLC system (Thermo Fisher Scientific, Waltham, MA, USA). The Waters ACQUITY UPLC HSS T3 Column (100 Å, 1.8 μm, 2.1 mm×100 mm, SKU: 186003539, Waters Corporation, Milford, MA, USA) was used for the separation of various compounds in the samples. Chromatography was done at 40° C. with a flow rate at 0.4 mL/min. A mobile phase consisting of acetonitrile (A) and water with 1% formic acid (B) was used. The column was initially equilibrated with 15% A and 85% B for 1 min. Then a gradient was performed from 15% A and 85% B to 50% A and 50% B (1-4 min), followed by a gently linear gradient from 50% A and 50% B to 65% A and 35% B (4-14 min), and after a further increased to 100% A within 2 min (14-16 min), the mobile phase was returned to the initial condition for another 4 min (16-20 min). Reference compounds for establishing standard curves included mulberroside A, trans-resveratrol and oxyresveratrol procured from Cayman (Ann Arbor, MI, USA), whereas moracin M and moracin C were procured from 1PlusChem (San Diego, CA). Arachidin-2 standard (>95% purify determined by HPLC under absorbance at 320 nm and 340 nm) was purified from elicited peanut hairy root culture medium as described before [7]. Dilutions of the standards were made in MeOH to obtain calibration curves for quantitative analysis, with the exception of mulberroside A which was dissolved in 95% ethanol. Calibration curves were established using absorbance at 320 nm for all compounds.

### 4.6. Liquid Chromatography-Mass Spectrometry Analysis

The UltiMate 3000 ultra-high-performance liquid chromatography (UHPLC) system (Thermo Scientific, Waltham, MA, USA) was used for chromatography. The chromatographic separation method followed the same HPLC conditions as described above, except 0.5% formic acid was used. The column temperature was maintained at 40° C. The flow rate was 0.4 mL/min, and the injection volume was 10 μL. UV chromatograms were recorded at 265 and 320 nm. Mass spectrometry was performed on a LTQ XL linear ion trap mass spectrometer (Thermo Scientific) with an electrospray ionization (ESI) source. Ultrahigh pure helium (He) was used as the collision gas and high purity nitrogen (N2) as the sheath and auxiliary gas. All mass spectra were performed in both positive and negative ion modes with ion spray voltage at 4 kV, sheath gas at 45 arbitrary units (AU), auxiliary gas at 15 AU, and capillary temperature at 300° C. Full mass scans were recorded in the range m/z 100-2000. Collision-induced dissociation (CID) was used for the breakage of the molecular ion into smaller fragments. The relative collision energy was set at 35% of the maximum to produce optimum yields of fragment ions. The data were analyzed using the Xcalibur software (Thermo Scientific, Waltham, MA, USA).

### 5. Conclusions

To our knowledge, this is the first report on the substantial production of specialized metabolites in chemically-elicited hairy root cultures of M. alba. The established hairy root cultures are a sustainable and reproducible source for known biologically active stilbenes and benzofurans, especially resveratrol, oxyresveratrol, mulberroside A, moracin C, moracin M, and chalcomoracin. The majority of these compounds were secreted into the culture medium, facilitating their extraction and analyses. Several other compounds were also induced and secreted into the culture medium holding the potential for discovery of novel bioactive compounds.

### REFERENCES

Nam, S.; Jang, H. W.; Shibamoto, T. Antioxidant activities of extracts from teas prepared from medicinal plants, Morus alba L., Camellia sinensis L., and Cudrania tricuspidata, and their volatile components. J. Agric. Food Chem. 2012, 60, 9097-9105.
Khan, M. A.; Rahman, A. A.; Islam, S.; Khandokhar, P.; Parvin, S.; Islam, M. B.; Hossain, M.; Rashid, M.; Sadik, G.; Nasrin, S.; Mollah, M. N. H.; Alam, A. H. M. K. A comparative study on the antioxidant activity of methanolic extracts from different parts of Morus alba L. (Moraceae). BMC Res. Notes 2013, 6, 24-28.
Zhishen, J.; Mengcheng, T.; Jianming, W. The determination of flavonoid contents in mulberry and their scavenging effects on superoxide radicals. Food Chem. 1999, 64, 555-559.
Sheng, Y.; Zheng, S.; Ma, T.; Zhang, C.; Ou, X.; He, X.; Xu, W.; Huang, K. Mulberry leaf alleviates streptozotocin-induced diabetic rats by attenuating NEFA signaling and modulating intestinal microflora. Sci. Rep. 2017, 7, 12041.
Kobayashi, Y.; Miyazawa, M.; Kamei, A.; Abe, K.; Kojima, T. Ameliorative effects of mulberry (Morus alba L.) leaves on hyperlipidemia in rats fed a high-fat diet: induction of fatty acid oxidation, inhibition of lipogenesis, and suppression of oxidative stress. Biosci. Biotechnol. Biochem. 2010, 74, 2385-2395.
Yang, N.-C.; Jhou, K.-Y.; Tseng, C.-Y., Antihypertensive effect of mulberry leaf aqueous extract containing y-aminobutyric acid in spontaneously hypertensive rats. Food Chem. 2012, 132, 1796-1801.
Yang, Y.; Tan, Y. X.; Chen, R. Y.; Kang, J. The latest review on the polyphenols and their bioactivities of Chinese Morus plants. J. Asian Nat. Prod. Res. 2014, 16, 690-702.
Akinwumi, B. C.; Bordun, K. A. M.; Anderson, H. D. Biological Activities of Stilbenoids. Int. J. Mol. Sci. 2018, 19, 792.
Kim, Y. J.; Chung, S. O.; Kim, J. K.; Park, S. U. Recent studies on resveratrol and its biological and pharmacological activity. EXCLI J. 2017, 16, 602-608.
Lorenz, P.; Roychowdhury, S.; Engelmann, M.; Wolf, G.; Horn, T. F. Oxyresveratrol and resveratrol are potent antioxidants and free radical scavengers: Effect on nitrosative and oxidative stress derived from microglial cells. Nitric Oxide 2003, 9, 64-76.
Chen, Y.-C.; Tien, Y.-J.; Chen, C.-H.; Beltran, F. N.; Amor, E. C.; Wang, R.-J.; Wu, D.-J.; Mettling, C.; Lin, Y.-L.; Yang, W.-C. Morus alba and active compound oxyresveratrol exert anti-inflammatory activity via inhibition of leukocyte migration involving MEK/ERK signaling. BMC Compl. Alternative Med. 2013, 13, 45.
Hwang, D.; Jo, H.; Kim, J.-K.; Lim, Y.-H. Oxyresveratrol-containing Ramulus mori ethanol extract attenuates acute colitis by suppressing inflammation and increasing mucin secretion. J. Funct. Foods 2017, 35, 146-158.
Sasivimolphan, P.; Lipipun, V.; Likhitwitayawuid, K.; Takemoto, M.; Pramyothin, P.; Hattori, M.; Shiraki, K. Inhibitory activity of oxyresveratrol on wild-type and drug-resistant varicella-zoster virus replication in vitro. Antiviral Res. 2009, 84, 95-97.
Ban, J. Y.; Jeon, S. Y.; Nguyen, T. T.; Bae, K.; Song, K. S.; Seong, Y. H. Neuroprotective effect of oxyresveratrol from Smilacis chinae rhizome on amyloid Beta protein (25-35)-induced neurotoxicity in cultured rat cortical neurons. Biol. Pharm. Bull. 2006, 29, 2419-2424.
Li, H.; Cheng, K.-W.; ChO, C.-H.; He, Z.; Wang, M. Oxyresveratrol as an antibrowning agent for cloudy apple juices and fresh-cut apples. J. Agric. Food Chem. 2007, 55, 2604-2610.
Shin, N. H.; Ryu, S. Y.; Choi, E. J.; Kang, S. H.; Chang, I. M.; Min, K. R.; Kim, Y. Oxyresveratrol as the potent inhibitor on dopa oxidase activity of mushroom tyrosinase. Biochem. Biophys. Res. Commun. 1998, 243, 801-803.
Kim, J. H.; Park, K. T.; Lee, H. S.; Kim, M.; Lim, Y. H. Evaluation of the inhibition of mushroom tyrosinase and cellular tyrosinase activities of oxyresveratrol: Comparison with mulberroside A. J. Enzym. Inhib. Med. Chem. 2012, 27, 495-503
Kim, Y. M.; Yun, J.; Lee, C.-K.; Lee, H.; Min, K. R.; Kim, Y. Oxyresveratrol and hydroxystilbene compounds: Inhibitory effect on tyrosinase and mechanism of action. J. Biol. Chem. 2002, 277, 16340-16344.
Kim, J. K.; Kim, M.; Cho, S. G.; Kim, M. K.; Kim, S. W.; Lim, Y. H. Biotransformation of mulberroside A from Morus alba results in enhancement of tyrosinase inhibition. J. Ind. Microbiol. Biotechnol. 2010, 37, 631-637.
Guo, F.; Zou, Y.; Zheng, Y. Moracin M inhibits lipopolysaccharide-induced inflammatory responses in nucleus pulposus cells via regulating PI3K/Akt/mTOR phosphorylation. Int. Immunopharmacol. 2018, 58, 80-86.
Chen, S. K.; Zhao, P.; Shao, Y. X.; Li, Z.; Zhang, C.; Liu, P.; He, X.; Luo, H. B.; Hu, X., Moracin M from Morus alba L. is a natural phosphodiesterase-4 inhibitor. Bioorg. Med. Chem. Lett. 2012, 22, 3261-3264.
Takasugi, M.; Nagao, S.; Ueno, S.; Masamune, T.; Shirata, A.; Takahashi, K. Moracin C and D, new phytoalexins from diseased mulberry. Chem. Lett. 1978, 7, 1239-1240.
Yao, X.; Wu, D.; Dong, N.; Ouyang, P.; Pu, J.; Hu, Q.; Wang, J.; Lu, W.; Huang, J. Moracin C, a phenolic compound isolated from Artocarpus heterophyllus, suppresses lipopolysaccharide-activated inflammatory responses in murine raw264. 7 macrophages. Int. J. Mol. Sci. 2016, 17, 1199.
Kim, Y. J.; Sohn, M. J.; Kim, W. G. Chalcomoracin and moracin C, new inhibitors of Staphylococcus aureus enoyl-acyl carrier protein reductase from Morus alba. Biol. Pharm. Bull. 2012, 35, 791-795.
Zelefack, F.; Guilet, D.; Valentin, A.; Fongang, R. C. S.; Kom, B.; Chevalley. S.; Ngouela, S. A.; Tsamo, E.; Fabre, N.; Dijoux-Franca, M. G. Antiplasmodial and cytotoxic activities of flavonoids and arylbenzofuran derivatives from Morus mesozygia. Greener J. Biol. Sci. 2012, 2, 20-24.
Ferlinahayati; Syah, Y. M.; Juliawaty, L. D; Achmad, S. A.; Hakim, E. H.; Takayama, H.; Said, I. M.; Latip, J. Phenolic constituents from the wood of Morus australis with cytotoxic activity. Z. Naturforsch. C Biosci. 2008, 63, 35-39
Li, X.; Xie, H.; Zhan, R.; Chen, D. Effect of double bond position on 2-phenyl-benzofuran antioxidants: a comparative study of moracin C and iso-moracin C. Molecules 2018, 23, 754.
Yang, Z.; Wang, Y.; Wang, Y.; Zhang, Y. Bioassay-guided screening and isolation of a-glucosidase and tyrosinase inhibitors from leaves of Morus alba. Food Chem. 2012, 131, 617-625.
Takasugi, M.; Nagao, S.; Masamune, T.; Shirata, A.; Takahashi, K. Chalcomoracin, a natural Diels-Alder adduct from diseased mulberry. Chem. Lett. 1980, 9, 1573-1576.
Fukai, T.; Oku, Y.; Hano, Y.; Terada, S. Antimicrobial activities of hydrophobic 2-arylbenzofurans and an isoflavone against vancomycin-resistant enterococci and methicillin-resistant Staphylococcus aureus. Planta Med. 2004, 70, 685-687.
Fukai, T.; Kaitou, K.; Terada, S. Antimicrobial activity of 2-arylbenzofurans from Morus species against methicillin-resistant Staphylococcus aureus. Fitoterapia 2005, 76, 708-711.
Zhang, Q. J.; Tang, Y. B.; Chen, R. Y.; Yu, D. Q. Three new cytotoxic Diels-Alder-type adducts from Morus australis. Chem. Biodivers. 2007, 4, 1533-1540.
Han, H.; Chou, C.-C.; Li, R.; Liu, J.; Zhang, L.; Zhu, W.; Hu, J.; Yang, B.; Tian, J. Chalcomoracin is a potent anticancer agent acting through triggering oxidative stress via a mitophagy- and paraptosis-dependent mechanism. Sci. Rep. 2018, 8, 9566.
Guillon, S.; Tremouillaux-Guiller, J.; Pati, P. K.; Rideau, M.; Gantet, P. Hairy root research: recent scenario and exciting prospects. Curr. Opin. Plant Biol. 2006, 9, 341-346.
Gutierrez-Valdes, N.; Hakkinen, S. T.; Lemasson, C.; Guillet, M.; Oksman-Caldentey, K.-M.; Ritala, A.; Cardon, F. Hairy root cultures-A versatile tool with multiple applications. Front. Plant Sci. 2020, 11, 33
Fang, L.; Yang, T.; Medina-Bolivar, F. Production of prenylated stilbenoids in hairy root cultures of peanut (Arachis hypogaea) and its wild relatives A. ipaensis and A. duranensis via an optimized elicitation procedure. Molecules 2020, 25, 509.
Nilsson, O.; Olsson, O. Getting to the root: The role of the Agrobacterium rhizogenes rol genes in the formation of hairy roots. Physiol. Plant. 1997, 100, 463-473
Jouanin, L.; Guerche, P.; Pamboukdjian, N.; Tourneur, C.; Delbart, F. C.; Tourneur, J. Structure of T-DNA in plants regenerated from roots transformed by Agrobacterium rhizogenes strain A4. Mol. Gen. Genet. 1987, 206, 387-392.
Jeon, Y. H.; Choi, S. W. Isolation, identification, and quantification of tyrosinase and a-glucosidase inhibitors from UVC-irradiated mulberry (Morus alba L.) leaves. Prev. Nutr. Food Sci. 2019, 24, 84-94.
Du, J.; He, Z. D.; Jiang, R. W.; Ye, W. C.; Xu, H. X.; Pui-Hay But, P. Antiviral flavonoids from the root bark of Morus alba L. Phytochemistry 2003, 62, 1235-1238.
Singab, A. N.; El-Beshbishy, H. A.; Yonekawa, M.; Nomura, T.; Fukai, T. Hypoglycemic effect of Egyptian Morus alba root bark extract: Effect on diabetes and lipid peroxidation of streptozotocin-induced diabetic rats. J. Ethnopharmacol. 2005, 100, 333-338.
Yang, T.; Fang, L.; Sanders, S.; Jayanthi, S.; Rajan, G.; Podicheti, R.; Medina-Bolivar, F. Stilbenoid prenyltransferases define key steps in the diversification of peanut phytoalexins. J. Biolog. Chem. 2018, 293, 28-46.
Gu, X. D.; Sun, M. Y.; Zhang, L.; Fu, H. W.; Cui, L.; Chen, R. Z.; Zhang, D. W.; Tian, J. K. UV-B induced changes in the secondary metabolites of Morus alba L. Leaves. Molecules 2010, 15, 2980-2993.
Oka, S.; Tewary, P. K. Induction of hairy roots from hypocotyls of mulberry (Morus indica L.) by Japanese wild strains of Agrobacterium rhizogenes. J. Seric. Sci. Jpn. 2000, 69, 13-19.
Nguyen, H. T. M.; Tran, T. Q.; Bui, A. L.; Quach, P. N. D. Hairy root culture of white mulberry (Morus alba L.) for a source of tyrosinase inhibitors. J. Biol. Res.2020, 93.
Jinhong, Y. Establishment of plantlet regeneration conditions for mulberry hairy root. J. Henan Agricultural Sci. 2013, 42, 113-115.
Giri, C. C.; Zaheer, M. Chemical elicitors versus secondary metabolite production in vitro using plant cell, tissue and organ cultures: Recent trends and a sky eye view appraisal. Plant Cell Tissue Organ Cult. 2016, 126, 1-18.
Ho, T.-T.; Murthy, H. N.; Park, S.-Y. Methyl jasmonate induced oxidative stress and accumulation of secondary metabolites in plant cell and organ cultures. Int. J. Mol. Sci. 2020, 21, 716.
Bru, R.; Selles, S.; Casado-Vela, J.; Belchi-Navarro, S.; Pedreño, M. A. Modified cyclodextrins are chemically defined glucan inducers of defense responses in grapevine cell cultures. J. Agric. Food Chem. 2006, 54, 66-71.
Zamboni, A.; Gatto, P.; Cestaro, A.; Pilati, S.; Viola, R.; Mattivi, F.; Moser, C.; Velasco, R. Grapevine cell early activation of specific responses to DIMEB, a resveratrol elicitor. BMC Genomics 2009, 10, 363.
Almagro, L.; Belchi-Navarro, S.; Martinez-Marquez, A.; Bru, R.; Pedreno, M. A. Enhanced extracellular production of trans-resveratrol in Vitis vinifera suspension cultured cells by using cyclodextrins and coronatine. Plant Physiol. Biochem. 2015, 97, 361-367.
Gill, S. S.; Tuteja, N. Reactive oxygen species and antioxidant machinery in abiotic stress tolerance in crop plants. Plant Physiol. Biochem. 2010, 48, 909-930.
Ahmad, I.; Basra, S. M. A.; Wahid, A. Exogenous application of ascorbic acid, salicylic acid and hydrogen peroxide improves the productivity of hybrid maize at low temperature stress. Int. J. Agric. Biol. 2014, 16, 825-830.
işeri, Ö.D.; Körpe, D. A.; Sahin, F. I.; Haberal, M. Hydrogen peroxide pretreatment of roots enhanced oxidative stress response of tomato under cold stress. Acta Physiol. Plant. 2013, 35, 1905-1913.
Li, J.-T.; Qiu, Z.-B.; Zhang, X.-W.; Wang, L.-S. Exogenous hydrogen peroxide can enhance tolerance of wheat seedlings to salt stress. Acta Physiol. Plant. 2011, 33, 835-842.
Ashfaque, F.; Khan, M. I. R.; Khan, N. A. Exogenously applied H₂O₂ promotes proline accumulation, water relations, photosynthetic efficiency and growth of wheat (Triticum aestivum L.) under salt stress. Annu. Res. Rev. Biol. 2014, 4, 105-120.
Repka, V., Fiala, R., Čiamporová, M., Pavlovkin, J. Effects of ZnC12 on ROS generation, plasma membrane properties, and changes in protein expression in grapevine root explants. Biologia 2016, 71, 528-537.
Fariduddin, Q.; Khan, T. A.; Yusuf, M. Hydrogen peroxide mediated tolerance to copper stress in the presence of 28-homobrassinolide in Vigna radiata. Acta Physiol. Plant. 2014, 36, 2767-2778.
Khan, M. I.; Khan, N. A.; Masood, A.; Per, T. S.; Asgher, M. Hydrogen peroxide alleviates nickel-inhibited photosynthetic responses through increase in use-efficiency of nitrogen and sulfur, and glutathione production in mustard. Front. Plant Sci. 2016, 7, 44.
Moomaw, A. S.; Maguire, M. E. The unique nature of Mg2+channels. Physiology 2008, 23, 275-285.
Liang, P. H.; Ko, T. P.; Wang, A. H. Structure, mechanism and function of prenyltransferases. Eur. J. Biochem. 2002, 269, 3339-3354.
Simons, R.; Gruppen, H.; Bovee, T. F.; Verbruggen, M. A.; Vincken, J.-P. Prenylated isoflavonoids from plants as selective estrogen receptor modulators (phytoSERMs). Food Funct. 2012, 3, 810-827.
Kamal, M.; Shakya, K. A.; Jawaid, T. Benzofurans: a new profile of biological activities. Int. J. Med. Pharm. Sci. 2011, 1, 1-15.
Miao, Y.-H.; Hu, Y.-H.; Yang, J.; Liu, T.; Sun, J.; Wang, X.-J. Natural source, bioactivity and synthesis of benzofuran derivatives. RSC Adv. 2019, 9, 27510-27540.
Wei, H.; Zhu, J. J.; Liu, X. Q.; Feng, W. H.; Wang, Z. M.; Yan, L. H. Review of bioactive compounds from root barks of Morus plants (Sang-Bai-Pi) and their pharmacological effects. Cogent Chem. 2016, 2, 1212320.
Inyai, C.; Yusakul, G.; Komaikul, J.; Kitisripanya, T.; Likhitwitayawuid, K.; Sritularak, B.; Putalun, W. Improvement of stilbene production by mulberry Morus alba root culture via precursor feeding and co-elicitation. Bioprocess Biosyst. Eng. 2021, 44, 653-660.
Komaikul, J.; Kitisripanya, T.; Tanaka, H.; Sritularak, B.; Putalun, W. Enhanced mulberroside a production from cell suspension and root cultures of Morus alba using elicitation. Nat. Prod. Commun. 2015, 10, 1253-1256.
Happyana, N.; Hakim, E. H.; Syah, Y. M.; Kayser, O.; Juliawaty, L. D.; Mujahidin, D.; Ermayanti, T. M.; Achmad, S. A. Diels-Alder type adducts from hairy root cultures of Morus macroura. Nat. Prod. Sci. 2019, 25, 233-237.
Condori, J.; Sivakumar, G.; Hubstenberger, J.; Dolan, M. C.; Sobolev, V. S.; Medina-Bolivar, L. F. Induced biosynthesis of resveratrol and the prenylated stilbenoids arachidin-1 and arachidin-3 in hairy root cultures of peanut: Effects of culture medium and growth stage. Plant Physiol. Biochem. 2010, 48, 310-318.
Medina-Bolivar, F.; Condori, J.; Rimando, A. M.; Hubstenberger, J.; Shelton, K.; O'Keefe, S. F.; Bennett, S.; Dolan, M. C. Production and secretion of resveratrol in hairy root cultures of peanut. Phytochemistry 2007, 68, 1992-2003.

Example 5 - Table 2 - Yield of the secreted compounds in the
culture medium of white mulberry hairy roots. The yields are in
mg/g DW of root tissue. Four biological replicates were performed.
Secreted Compounds in U-D2 Elicited Medium (mg/g DW of root tissue)

| | | | | 3′- | |
|---|---|---|---|---|---|
| | Oxyreaveratrol | Resveratrol | Moracin M | Prenylresveratrol | Moracin C |
| CD + MgCl2 + H2O2 + MeJA-48 hr | 1.15 ± 0.13 | 0.09 ± 0.02 | 4.14 ± 0.74 | 0.25 ± 0.08 | 1.82 ± 0.40 |
| CD + MgCl2 + H2O2 + MeJA-96 hr | 2.57 ± 0.50 | 0.13 ± 0.03 | 6.14 ± 1.48 | 0.27 ± 0.11 | 0.97 ± 0.34 |
| CD + MgCl2 + H2O2 + MeJA-144 hr | 4.75 ± 1.14 | 0.17 ± 0.13 | 6.62 ± 1.20 | 0.21 ± 0.25 | 0.44 ± 0.11 |
| CD + MgCl2 + H2O2 + MeJA-192 hr | 5.57 ± 2.64 | 0.24 ± 0.27 | 6.65 ± 1.26 | 0.17 ± 0.42 | 0.15 ± 0.08 |
| CD + MgCl2 + MeJA-48 hr | 0.73 ± 0.08 | 0.01 ± 0.00 | 1.79 ± 0.08 | 0.10 ± 0.03 | 0.66 ± 0.14 |
| CD + MgCl2 + MeJA-96 hr | 1.36 ± 0.21 | 0.03 ± 0.01 | 2.86 ± 0.10 | 0.04 ± 0.05 | 0.32 ± 0.06 |
| CD + MgCl2 + MeJA-144 hr | 1.44 ± 0.27 | 0.01 ± 0.01 | 2.71 ± 0.24 | — | 0.07 ± 0.05 |
| CD + MgCl2 + MeJA-192 hr | 1.21 ± 0.24 | 0.01 ± 0.01 | 2.25 ± 0.29 | — | — |
| CD + MgCl2 + H2O2-48 hr | 2.93 ± 0.62 | 0.18 ± 0.01 | 4.59 ± 0.86 | 1.37 ± 0.35 | 1.82 ± 0.65 |
| CD + MgCl2 + H2O2-96 hr | 6.27 ± 1.24 | 0.61 ± 0.16 | 7.30 ± 1.22 | 4.03 ± 2.10 | 1.01 ± 0.45 |
| CD + MgCl2 + H2O2-144 hr | 4.93 ± 1.38 | 0.56 ± 0.11 | 7.65 ± 1.14 | 5.00 ± 1.71 | 0.63 ± 0.39 |
| CD + MgCl2 + H2O2-192 hr | 4.82 ± 1.71 | 0.45 ± 0.13 | 7.82 ± 1.26 | 4.69 ± 1.01 | 0.49 ± 0.35 |

Example 6 - Table 3 - Yield of the secreted compounds in the
culture medium of white mulberry hairy roots. The yields are in
mg/L of culture medium. Four biological replicates were performed.
Secreted Compounds in U-D2 Elicited Medium (mg/L)

| | | | | 3′- | |
|---|---|---|---|---|---|
| | Oxyreaveratrol | Resveratrol | Moracin M | Prenylresveratrol | Moracin C |
| CD + MgCl2 + H2O2 + MeJA-48 hr | 1.84 ± 0.36 | 0.14 ± 0.01 | 6.47 ± 0.43 | 0.38 ± 0.07 | 2.84 ± 0.37 |
| CD + MgCl2 + H2O2 + MeJA-96 hr | 4.11 ± 1.19 | 0.20 ± 0.03 | 9.52 ± 0.83 | 0.40 ± 0.09 | 1.48 ± 0.26 |
| CD + MgCl2 + H2O2 + MeJA-144 hr | 7.41 ± 1.06 | 0.25 ± 0.13 | 10.34 ± 0.64 | 0.28 ± 0.29 | 0.69 ± 0.14 |
| CD + MgCl2 + H2O2 + MeJA-192 hr | 8.42 ± 2.33 | 0.34 ± 0.30 | 10.38 ± 0.71 | 0.19 ± 0.51 | 0.22 ± 0.08 |
| CD + MgCl2 + MeJA-48 hr | 1.57 ± 0.17 | 0.02 ± 0.01 | 3.85 ± 0.14 | 0.22 ± 0.08 | 1.41 ± 0.21 |
| CD + MgCl2 + MeJA-96 hr | 2.91 ± 0.39 | 0.05 ± 0.02 | 6.14 ± 0.24 | 0.08 ± 0.10 | 0.69 ± 0.11 |
| CD + MgCl2 + MeJA-144 hr | 3.09 ± 0.45 | 0.02 ± 0.01 | 5.81 ± 0.21 | — | 0.14 ± 0.10 |
| CD + MgCl2 + MeJA-192 hr | 2.59 ± 0.41 | 0.01 ± 0.01 | 4.82 ± 0.49 | — | — |
| CD + MgCl2 + H2O2-48 hr | 4.06 ± 1.49 | 0.25 ± 0.05 | 6.17 ± 1.64 | 1.80 ± 0.33 | 2.37 ± 0.59 |
| CD + MgCl2 + H2O2-96 hr | 8.68 ± 3.12 | 0.83 ± 0.30 | 9.66 ± 1.24 | 5.66 ± 3.08 | 1.28 ± 0.27 |
| CD + MgCl2 + H2O2-144 hr | 6.90 ± 2.94 | 0.75 ± 0.19 | 10.13 ± 1.07 | 6.51 ± 1.73 | 0.77 ± 0.30 |
| CD + MgCl2 + H2O2-192 hr | 6.81 ± 3.45 | 0.61 ± 0.19 | 10.33 ± 0.80 | 6.18 ± 0.94 | 0.61 ± 0.31 |

### Example 7—Decreasing Melanin and Tyrosinase Activity in Melanocytes for Decreasing Pigmentation

Human melanocytes from iPSC (Phanocell, France) were seeded in 24-well plates at 20,000 cells/cm² in the culture medium recommended by the supplier, and cultured for 4 days at 37° C. under 5% of CO2. Then the cells were incubated with the products to be tested, solubilized in a DMSO mother solution, and incubated in the same conditions for 3 days.

### Melanin Level:

After removal of the medium, and rinsing of the cells with Phosphate Buffer Saline (PBS), the cells were lysed with a solution of NaOH IN with 10% DMSO, and the quantity of melanin was determined by reading optical density (OD) at 475 nm.

### Tyrosinase Activity:

After removal of the medium, and rinsing of the cells with Phosphate Buffer Saline (PBS), the cells were gently stirred with a sodium phosphate buffer 0.1M pH 6.8 in presence of Triton X100, and centrifugated to recover the supernatant. The tyrosinase activity was determined using L-DOPA (Sigma-Aldrich, France) as substrate by measurement of the OD at 475 nm after 3 hours of incubation at 37° C.

The results are shown in Table 4. Melanin level and tyrosinase activity in the table are the average of 2 assays in triplicate.

TABLE 4

| | | | % of | % of tyrosinase |
|---|---|---|---|---|
| Tested product | Refence | Dose tested | melanin | activity |
| Control (no product | — | — | 100% | 100% |
| applied) | | | | |
| Dry hairy root | 1 | 100 μg/ml | 28% | 24% |
| culture medium | | | | |
| extract, elicited, | | | | |
| 96 hr, line U-D1 | | | | |
| Dry hairy root | 2 | 100 μg/ml | 4% | 3% |
| culture medium | | | | |
| extract, elicited, | | | | |
| 96 hr, line U-D2 | | | | |
| Dry hairy root | 3 | 100 μg/ml | 24% | 8% |
| culture medium | | | | |
| extract, elicited, | | | | |
| 96 hr, line U-A2 | | | | |
| Normal root (source | 4 | 100 μg/ml | 22% | 0% |
| Ukraine) culture | | | | |
| medium extract, | | | | |
| 96 hr, | | | | |

These results show that these extracts according to the invention decrease the melanin level and tyrosinase activity in human melanocytes.

### Example 8—Cosmetic Formulations

Exemplary cosmetic formulations are produced according to the formulations in Tables 5 and 6.

TABLE 5
Cosmetic Formulation

| | | | Weight |
|---|---|---|---|
| Phase | Trade name | INCI | % |
| A | Emulgade Sucro Plus | Sucrose Polystearate (and) | 1.00 |
| | | Cetyl Palmitate | |
| | Lanette O | Cetearyl Alcohol | 3.50 |
| | Cetiol C 5C | Coco-Caprylate/Caprate | 5.00 |
| | Myritol 318 RC | Caprylic/Capric Triglyceride | 3.00 |
| | Xiameter PMX-200 | Dimethicone | 0.50 |
| | Silicone Fluid 5 CS | | |
| | Cosmedia Ace | Sodium Polyacrylate (and) | 1.20 |
| | | Dicaprylyl Carbonate (and) | |
| | | Polyglyceryl-3 Caprate | |
| B | Eumulgin Prisma | Disodium Cetearyl | 0.20 |
| | | Sulfosuccinate | |
| | Rheocare XGN | Xanthan Gum | 10.00 |
| | (1% aqua Solution) | | |
| | Glycerin | Glycerin | 3.00 |
| | Water, demin. | Aqua | To 100 |
| C | 1,2-Pentanediol | Pentylene Glycol | 2.00 |
| | Euxyl PE 9010 | Phenoxyethanol (and) | 0.50 |
| | | Ethylhexylglycerin | |
| | Extract according to the | | 2.00 |
| | invention | | |

TABLE 6
Cosmetic Formulation

| | | Weight |
|---|---|---|
| Name | INCI | % |
| Emulgade ® SE-PF | Glyceryl Stearate, Ceteareth-20, | 6.00 |
| | Ceteareth-12, Cetearyl Alcohol, | |
| | Cetyl Palmitate | |
| Lanette ® O | Cetearyl Alcohol | 2.00 |
| Cetiol ® SN | Cetearyl Isononanoate | 3.00 |
| Isopropyl Myristate | Isopropyl Myristate | 5.00 |
| Cetiol ® Sensoft | Propylheptyl caprylate | 2.00 |
| Cetiol ® CC | Dicaprylyl Carbonate | 2.00 |
| Xiameter PMX-200 | Dimethicone | 1.00 |
| Silicone | | |
| Fluid 50 Cs (Dow | | |
| Corning) | | |
| Covi-ox ® T 70 C | Tocopherol | 0.20 |
| Elestab ™ 388 | Propylene Glycol, Phenoxyethanol, | 2.50 |
| | Chlorphenesin, Methylparaben | |
| Edeta ® BD | Disodium EDTA | 0.05 |
| 1,3-butanediol | Butylene Glycol | 3.00 |
| Rheocare ® XG | Xanthan Gum | 0.2 |
| Eumulgin ® SG | Sodium Stearoyl glutamate | 0.5 |
| NaOH | Sodium Hydroxide | 0.21 |
| Water | qsf 100.00 | qsf 100.00 |
| Extract according to the | — | 0.10 |
| invention | | |

### Example 9—Methods for Preparing a Botanical Composition

In one embodiment, the botanical composition can be prepared from an elicited hairy root culture of Morus alba, where the botanical composition comprises an extract of phenolic compounds from the hairy root tissue combined with an extract of phenolic compounds of the culture medium. To extract the phenolic compounds from the hairy root tissue after the elicitation period, the hairy roots can be first frozen and then lyophilized. The lyophilized hairy roots can then be grinded and extracted with a solvent to remove the phenolic compounds.

To extract the phenolic compounds from the culture medium, the medium can be mixed with a non-polar non-miscible solvent and then organic layer can be separated from the aqueous layer. The organic layer can then be dried under a stream of nitrogen or using a SpeedVac system. The dried culture medium extract can then be resuspended in a solvent.

To prepare the botanical composition, the extract from the hairy root tissue and the extract from the culture medium can be combined at equal or different ratios.

In another embodiment, the botanical composition can be prepared from an elicited hairy root culture of Morus alba, where the hairy root tissue and culture medium are lyophilized together and then placed in contact with a solvent to extract the phenolics compounds.

In another embodiment, the botanical composition can be prepared from an elicited normal root culture of Morus alba, where the botanical composition comprises an extract of phenolic compounds from the root tissue combined with an extract of phenolic compounds of the culture medium. To extract the phenolic compounds from the root tissue after the elicitation period, the roots can first be frozen and then lyophilized. The lyophilized roots can then be grinded and extracted with a solvent to remove the phenolic compounds.

To extract the phenolic compounds from the culture medium, the medium can be mixed with a non-polar non-miscible solvent and then organic layer can be separated from the aqueous layer. The organic layer can then be dried under a stream of nitrogen or using a SpeedVac system. The dried culture medium extract can then be resuspended in a solvent.

To prepare the botanical composition, the extract from the root tissue and the extract from the culture medium can be combined at equal or different ratios.

In another embodiment, the botanical composition can be prepared from an elicited normal root culture of Morus alba, where the root tissue and culture medium are lyophilized together and then placed in contact with a solvent to extract the phenolics compounds.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method for producing a botanical composition, the method comprising:
obtaining plant tissue from Morus alba;
producing a root culture from the plant tissue; and
stimulating production of a botanical composition from said root culture using an elicitation medium, wherein the elicitation medium comprises a combination of elicitors.

2. The method of claim 1, further comprising culturing the plant tissue for a period of time in a culture medium, such that the plant tissue produces a root culture.

3. The method of claim 2, wherein the time in the culture medium comprises less than 1 day, about 1 day, about 5 days, about 10 days, about 20 days, about 25 days, about 30 days, or more than 30 days.

4. The method of claim 1, further comprising incubating the root culture in the elicitation medium for about 4 hours, about 12 hours, about 24 hours, about 48 hours, about 96 hours, or about 192 hours.

5. The method of claim 1, wherein the root culture comprises root tissue, medium of said root culture, or a combination thereof.

6. The method of claim 5, further comprising the step of isolating the botanical composition from the root culture, thereby providing a botanical extract.

7. The method of claim 6, comprising contacting the root culture with a non-polar solvent and extracting the organic layer from the aqueous layer.

8. The method of claim 7, wherein the non-polar solvent comprises ethyl acetate.

9. The method of claim 6, comprising contacting the root culture with a polar solvent and extracting the organic layer from the aqueous layer.

10. The method of claim 9, wherein the polar solvent comprises water, an alcohol, a glycol, or a combination thereof.

11. The method of claim 10, wherein the alcohol comprises methanol, ethanol, propanol, butanol, pentanol, and hexanol.

12. The method of claim 10, wherein the glycol comprises ethylene glycol or propylene glycol.

13. The method of either claim 1 or claim 4, wherein the elicitors are selected from the group consisting of cyclodextrin, magnesium chloride, hydrogen peroxide, or a combination thereof.

14. The method of any one of claim 1, 4, or 13, wherein the elicitors can be present in a concentration comprising about 18 g/L cyclodextrin, about 1 mM magnesium chloride, about 3 mM hydrogen peroxide, or any combination thereof.

15. The method of claim 6, wherein the botanical composition comprises about 0.01 mg/g to about 50 mg/g of an aryl benzofuran compound or derivative thereof per dry weight of root tissue.

16. The method of claim 15, wherein the aryl benzofuran compound is a compound comprising Formula (I): 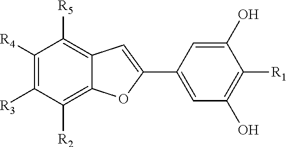
wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof.

17. The method of claim 16, wherein the compound comprising Formula (I) comprises: 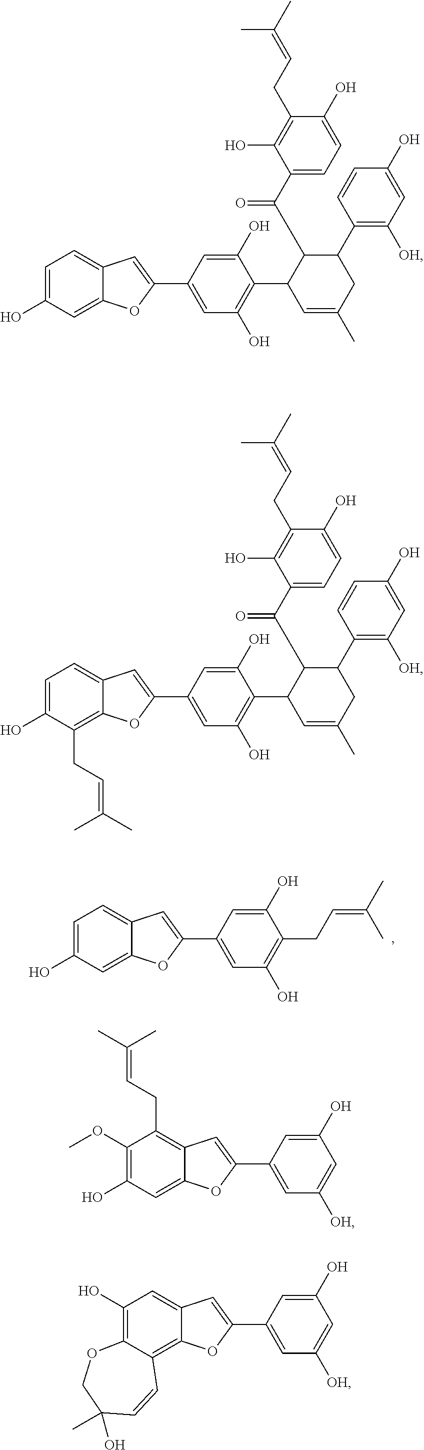 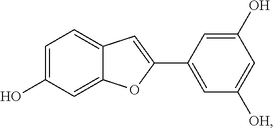 [SMILES: Oc1cc(O)cc(-c2cc3ccc(O)cc3o2)c1]
or
a glucosidyl derivative thereof.

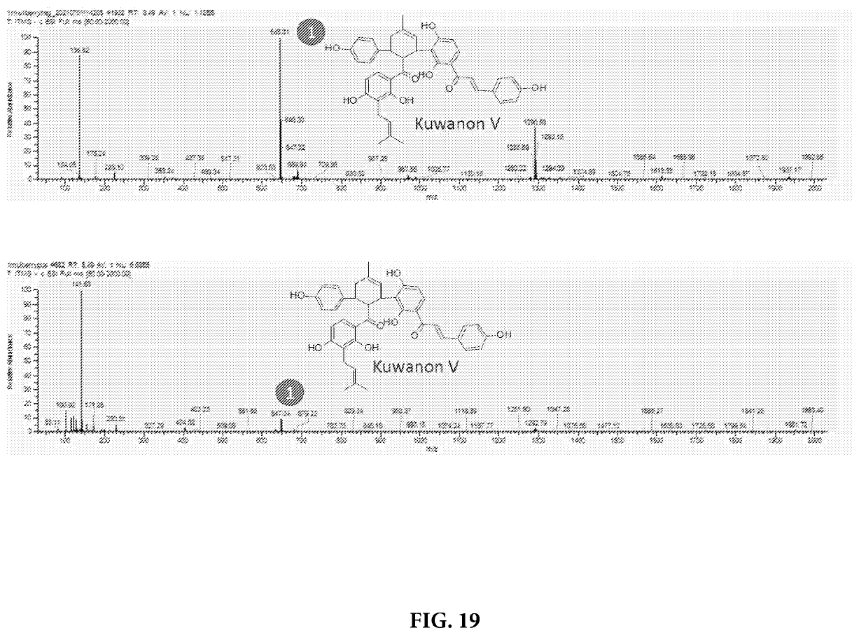

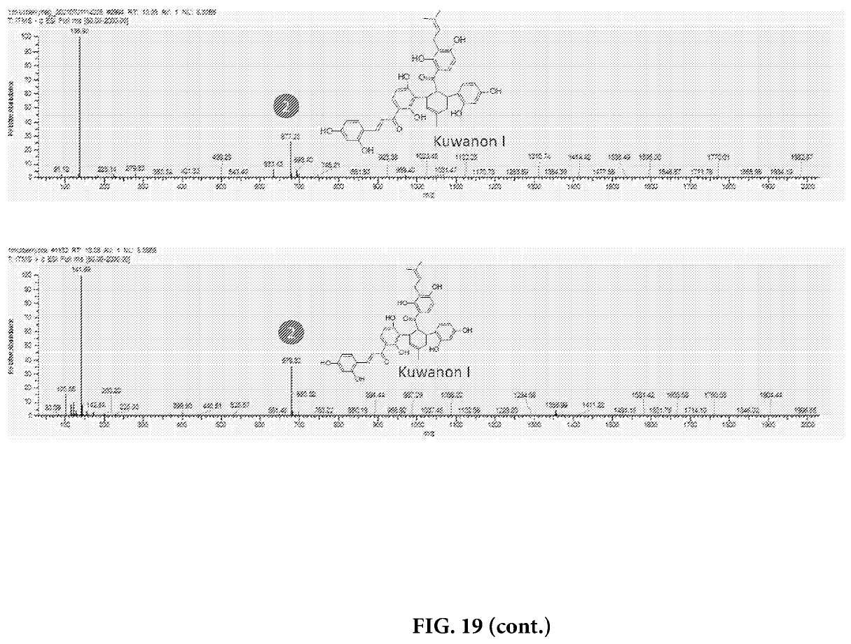

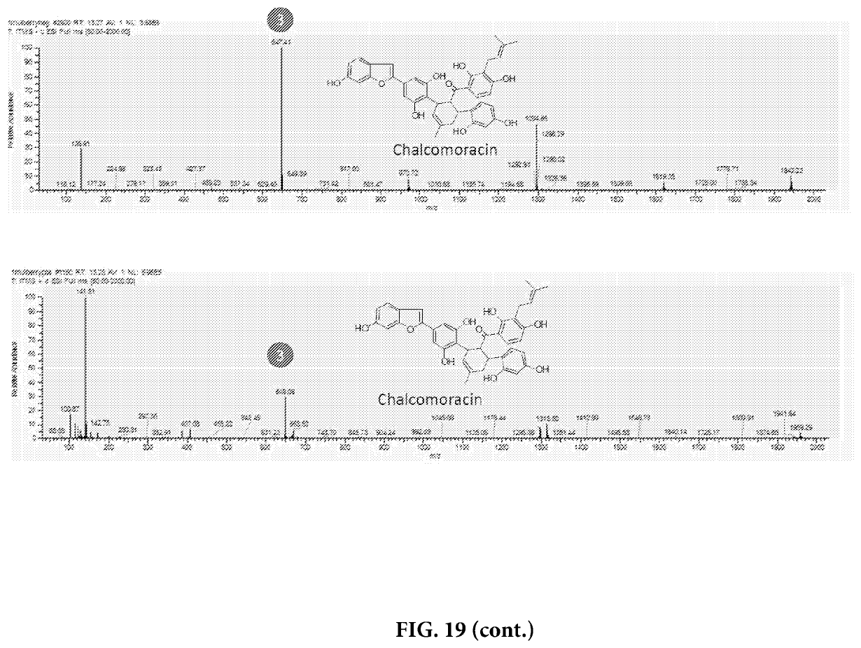

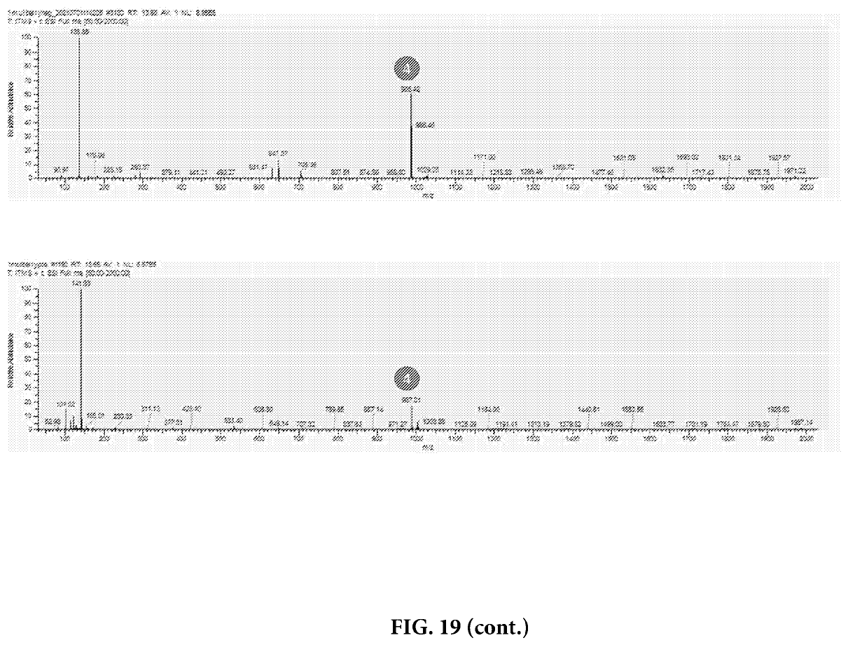

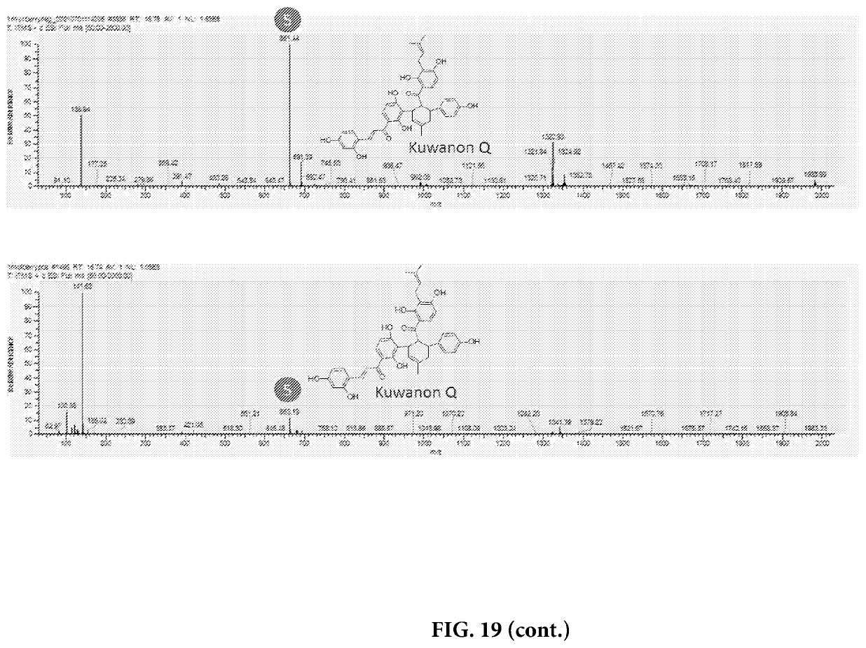

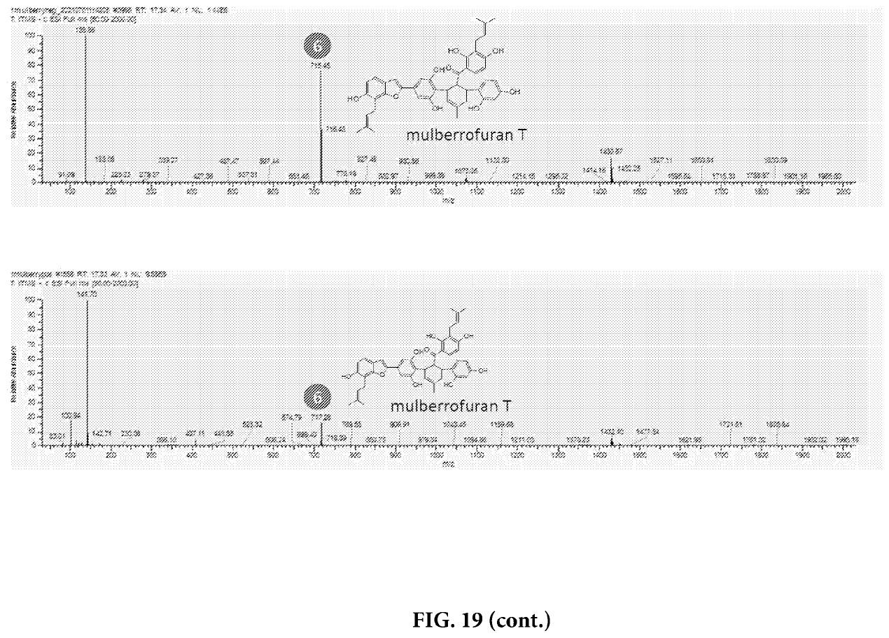

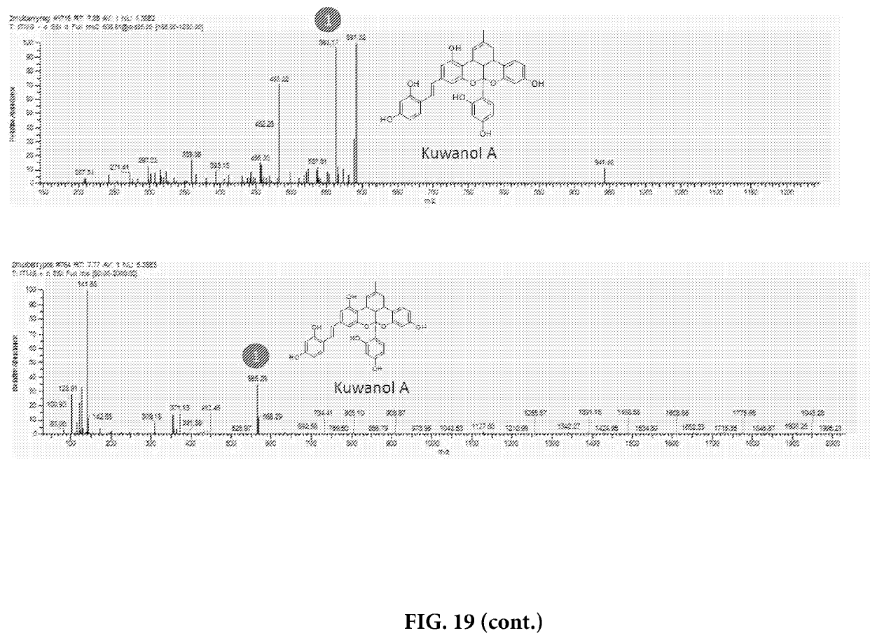

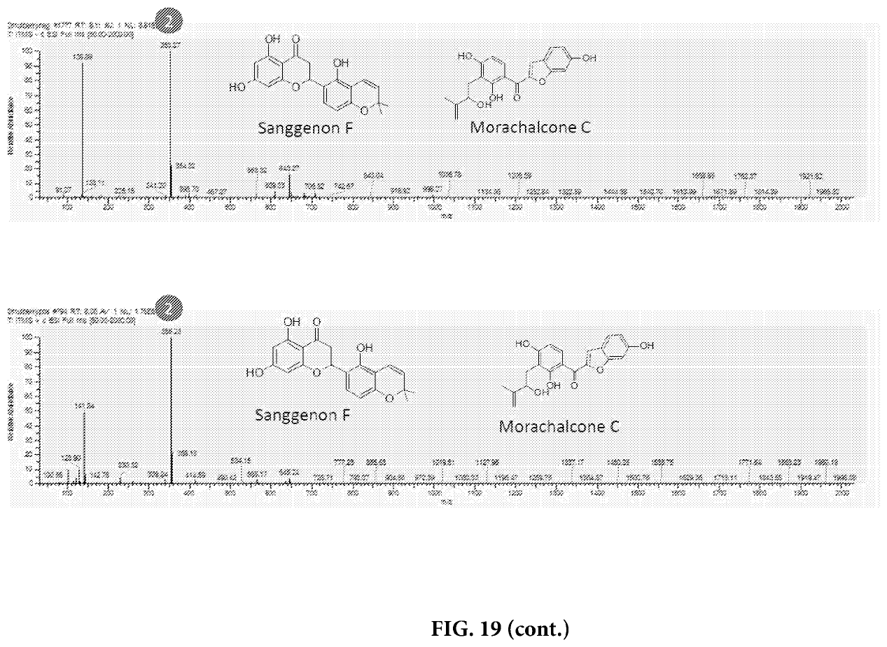

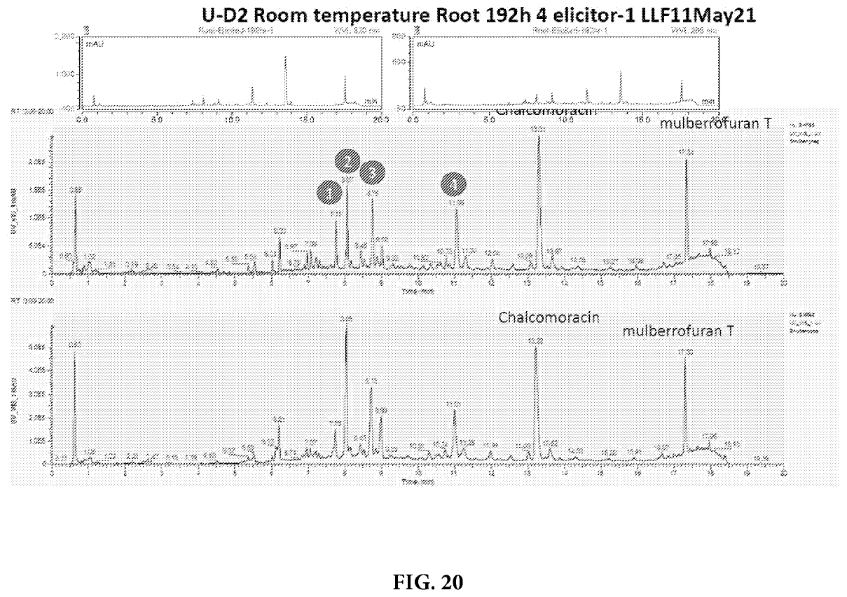

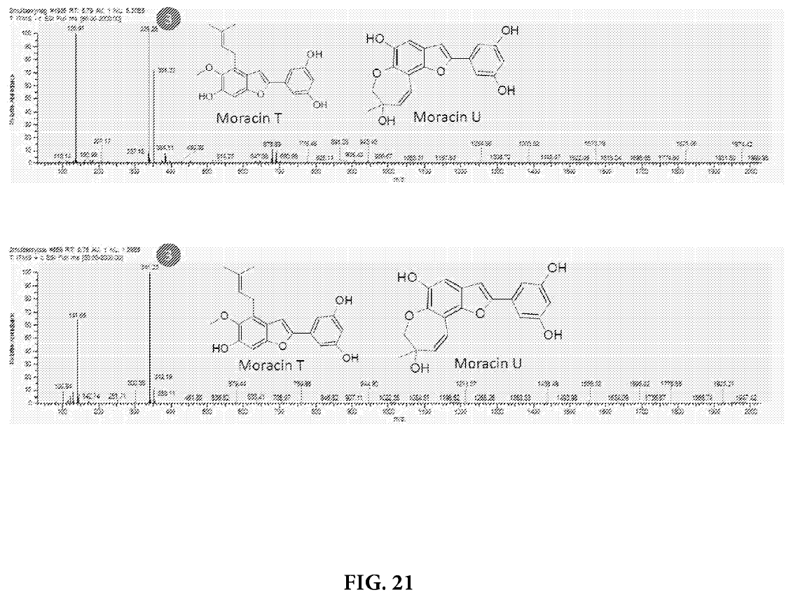

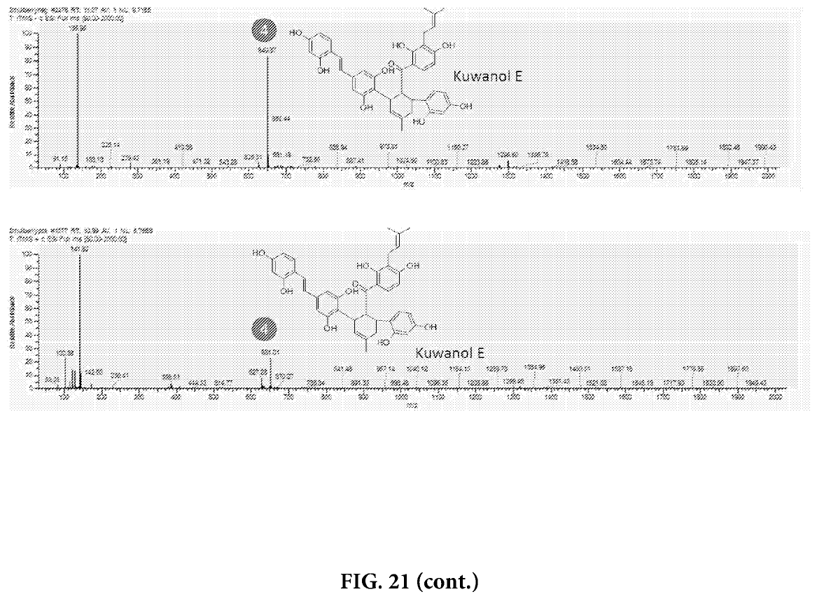

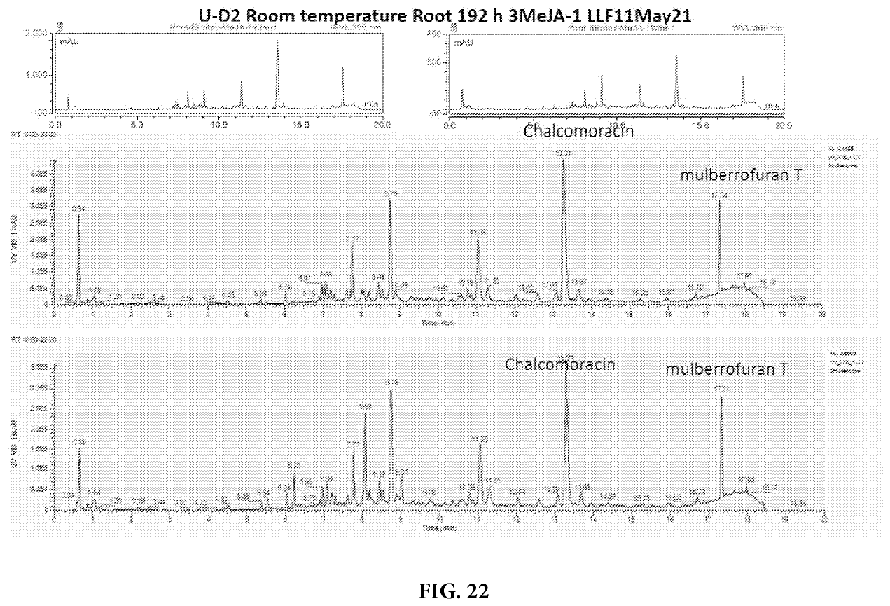

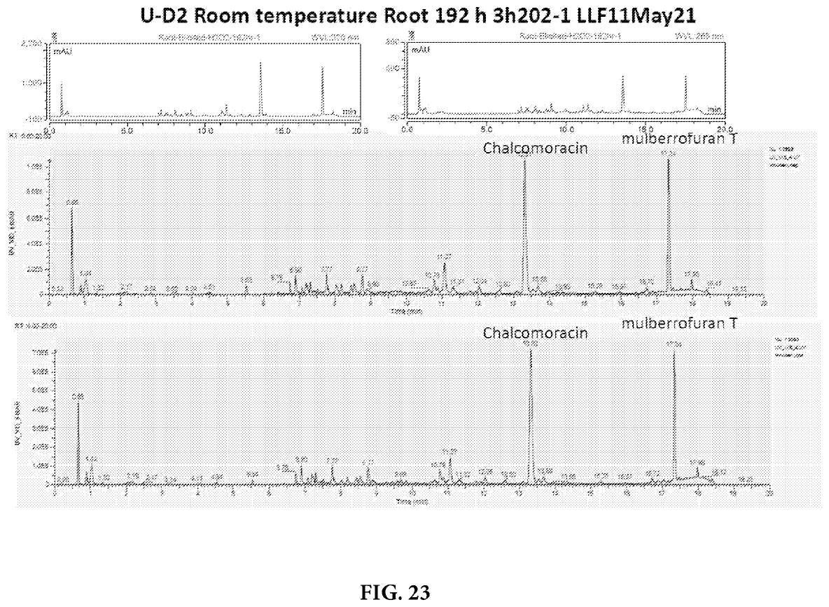

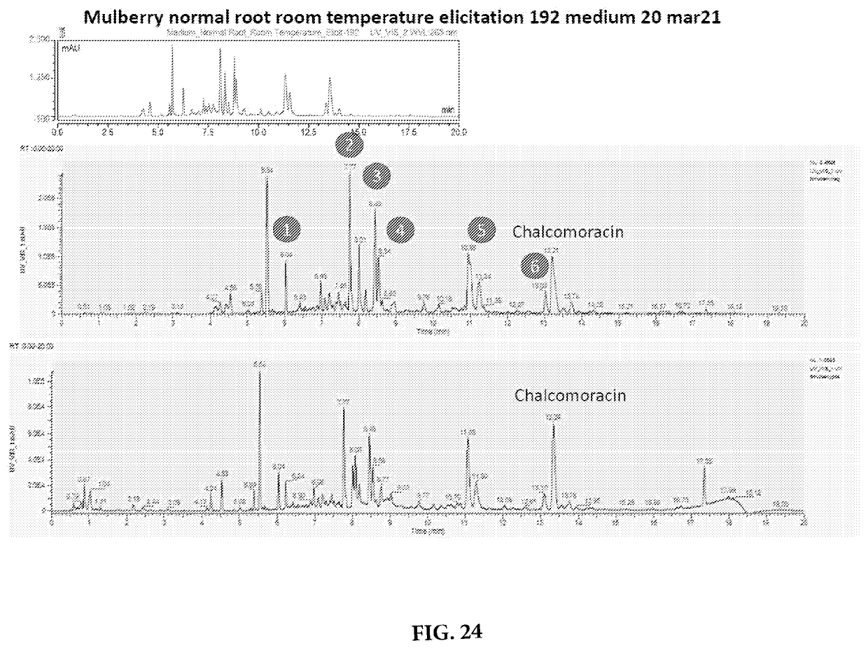

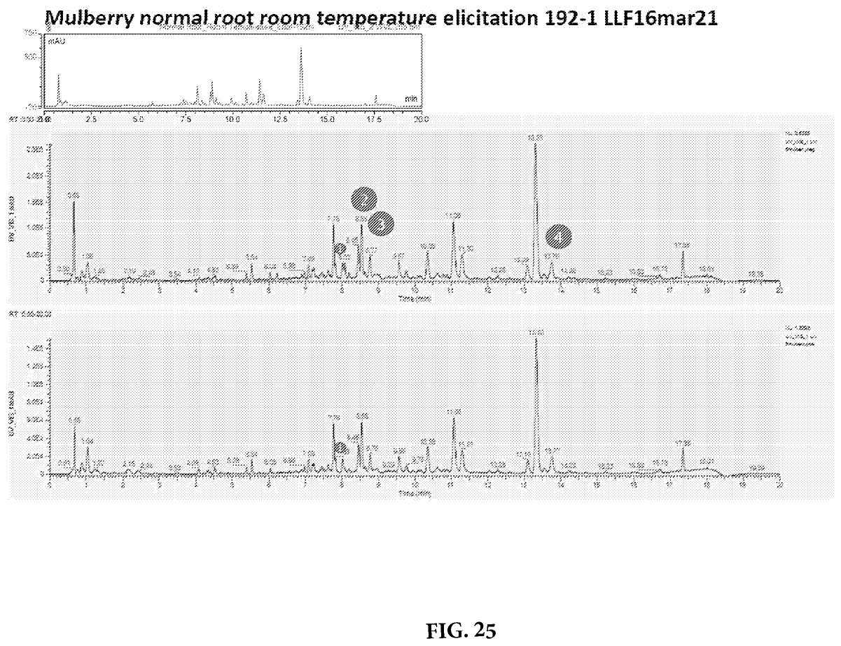

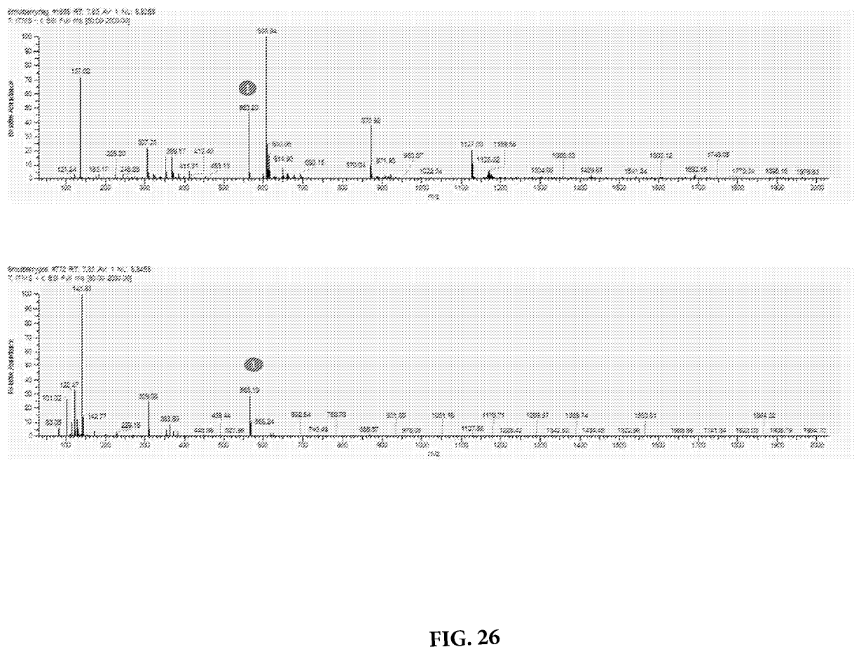

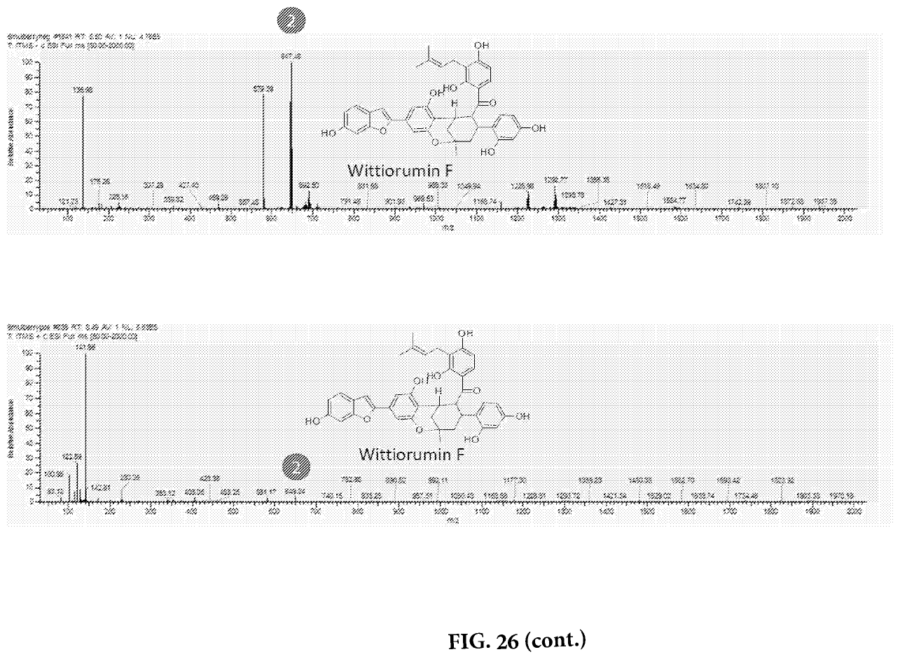

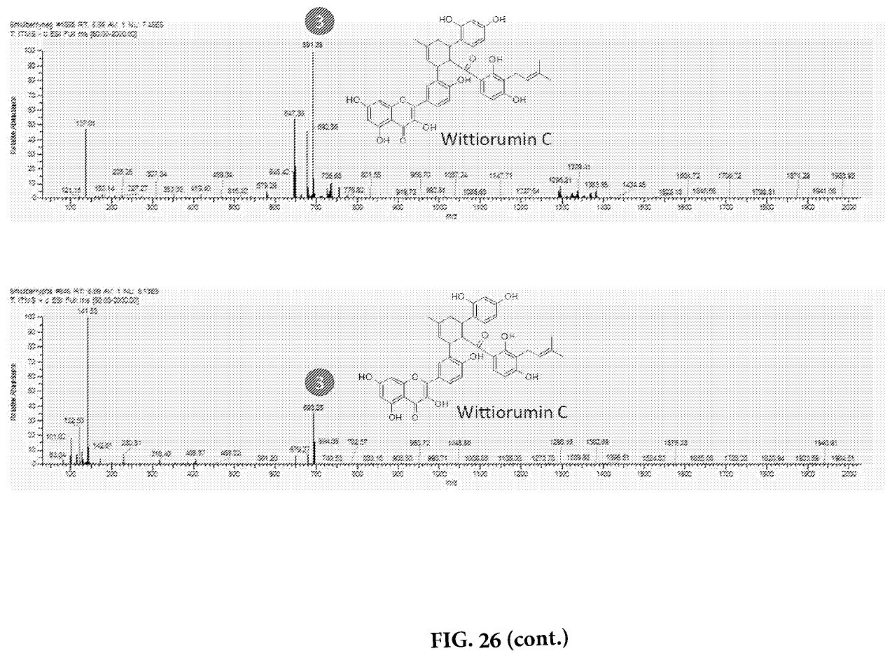

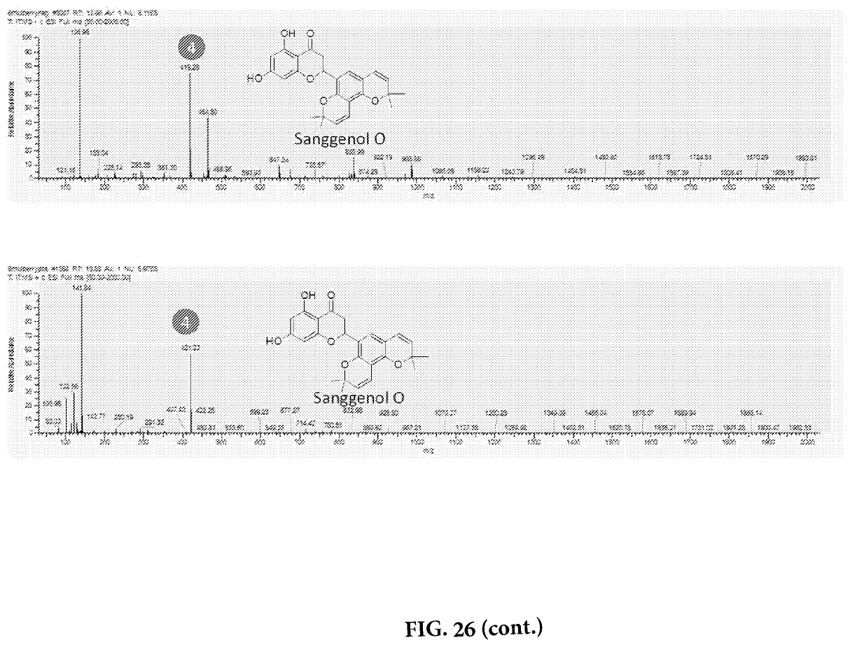

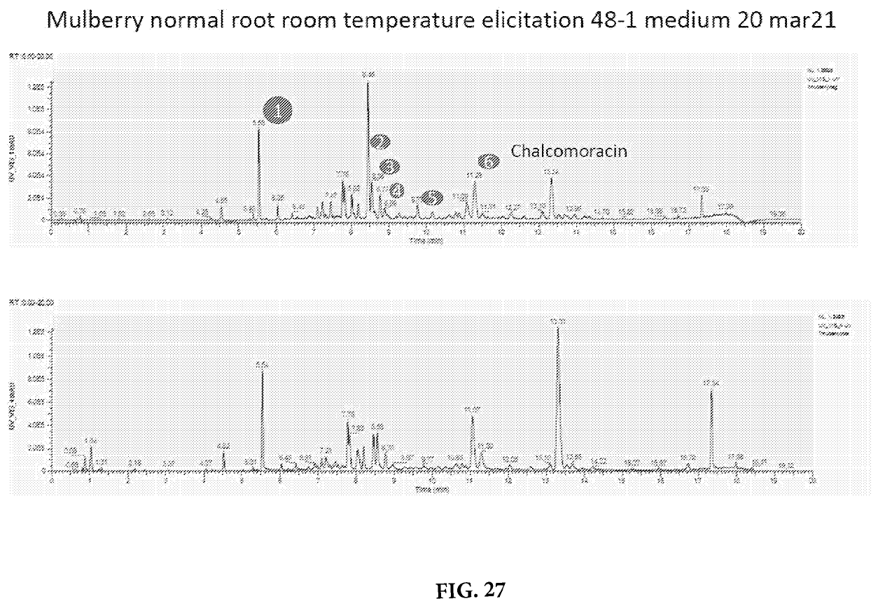

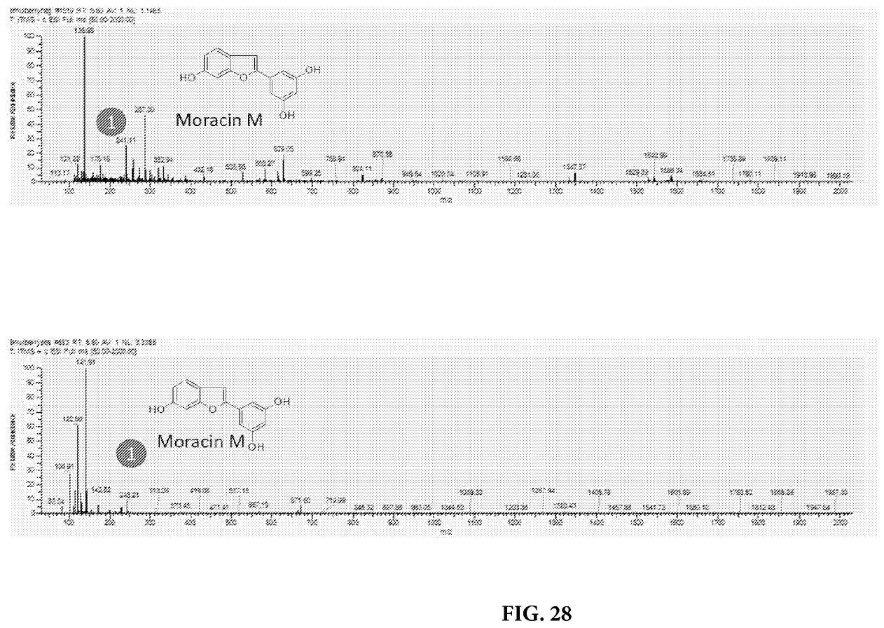

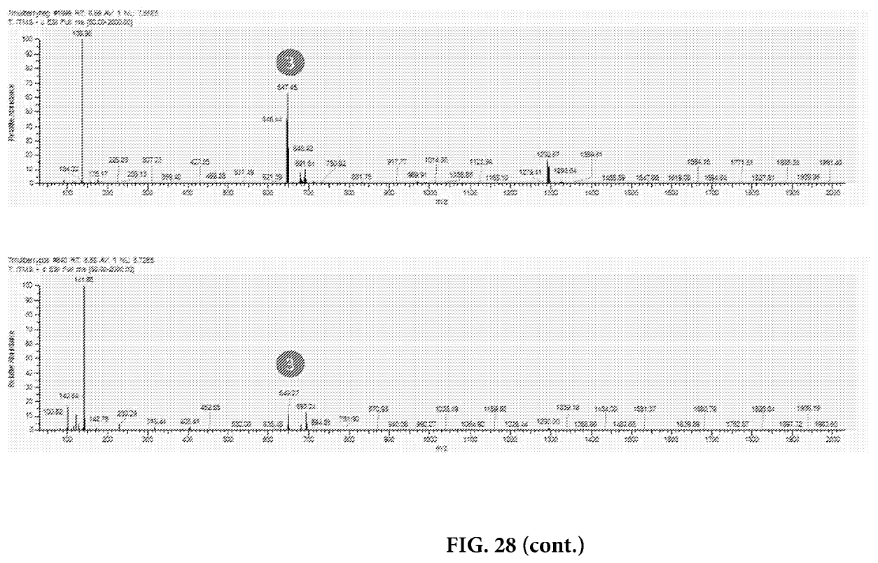

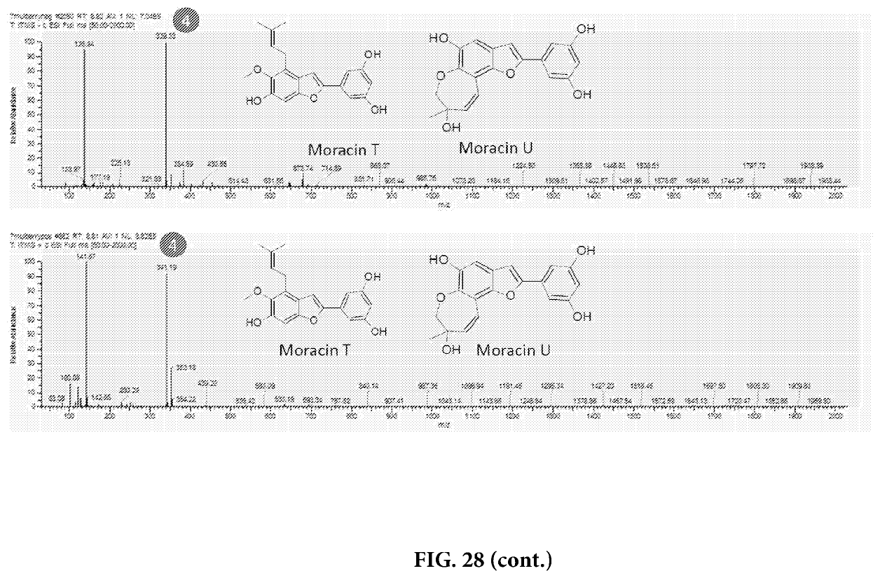

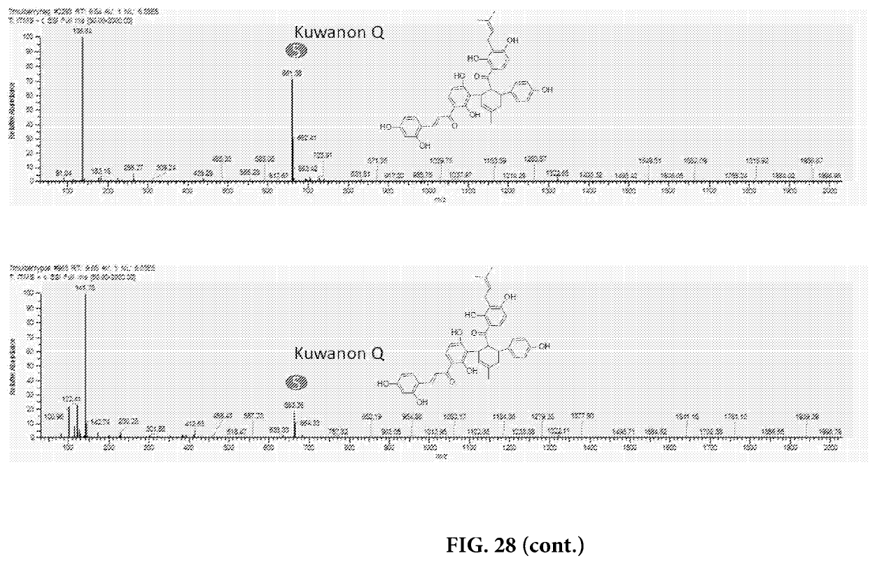

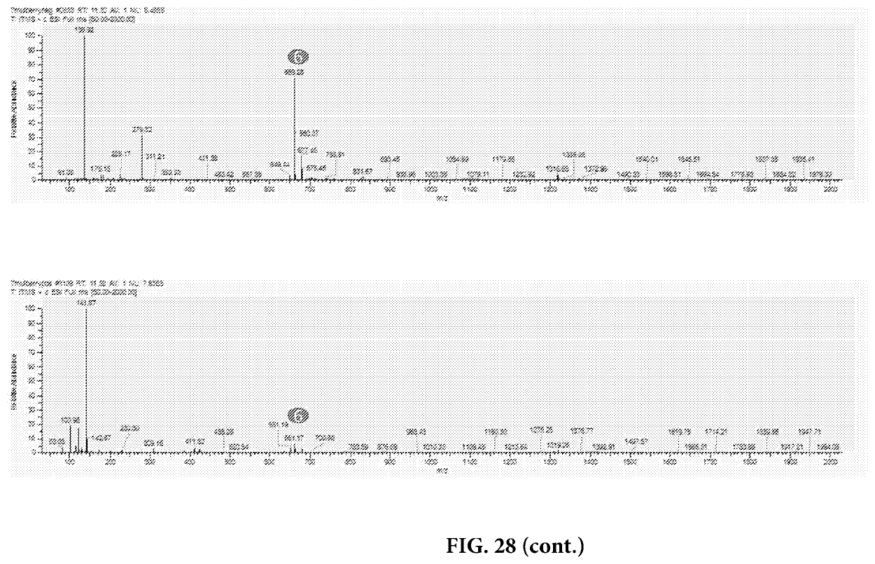

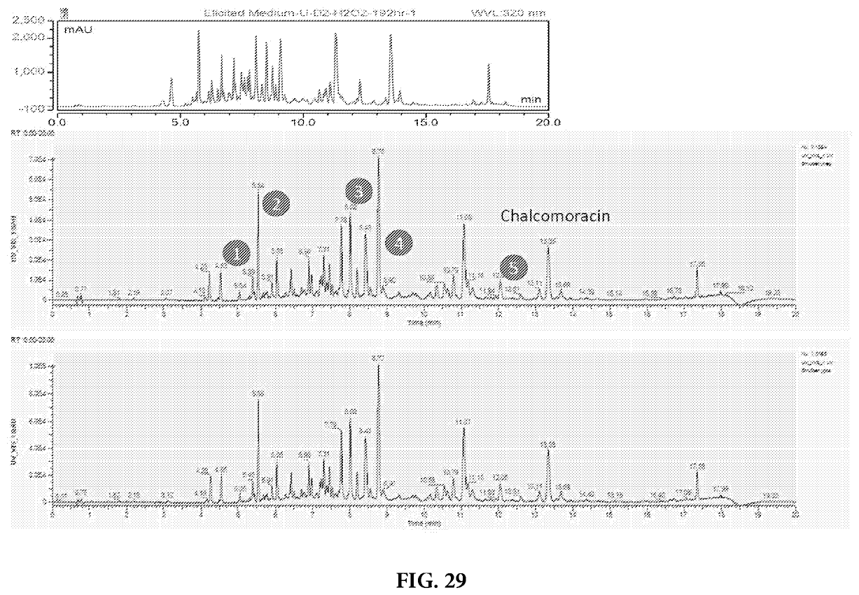

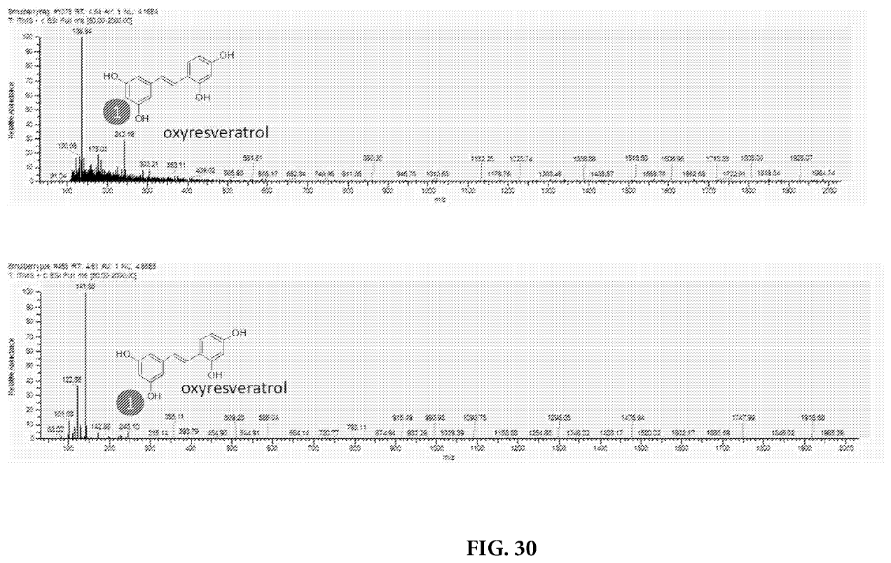

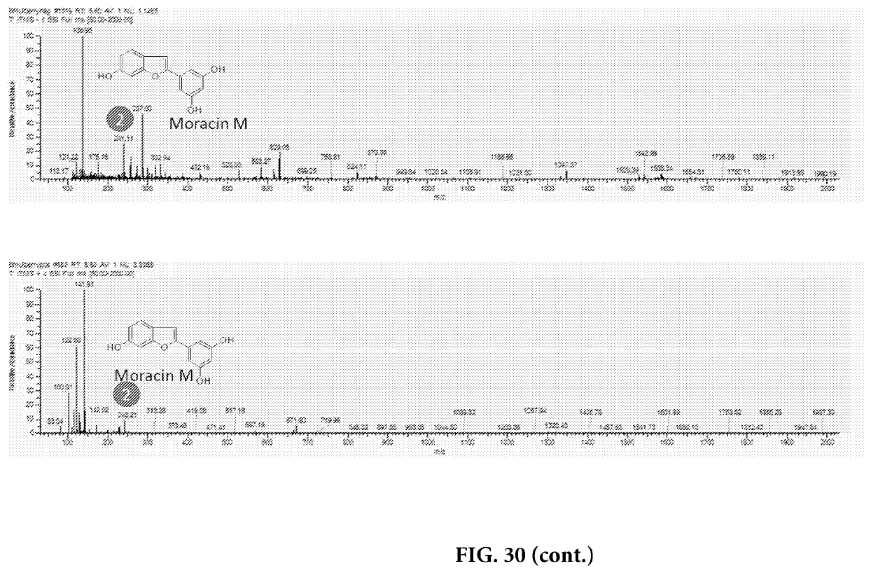

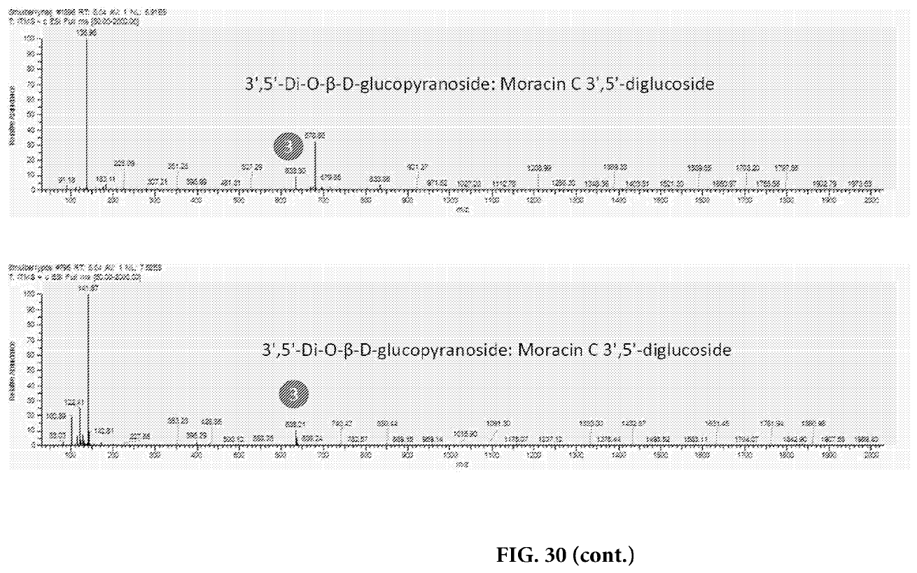

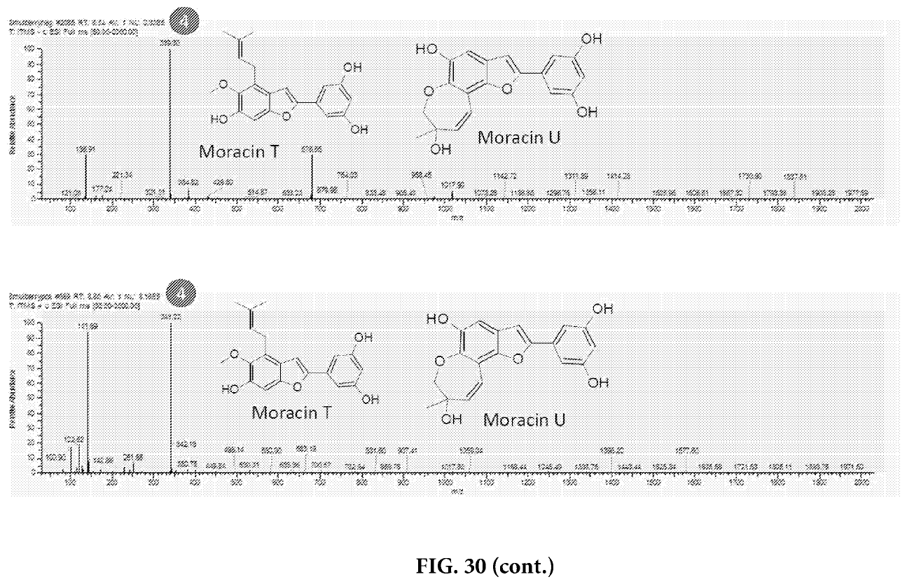

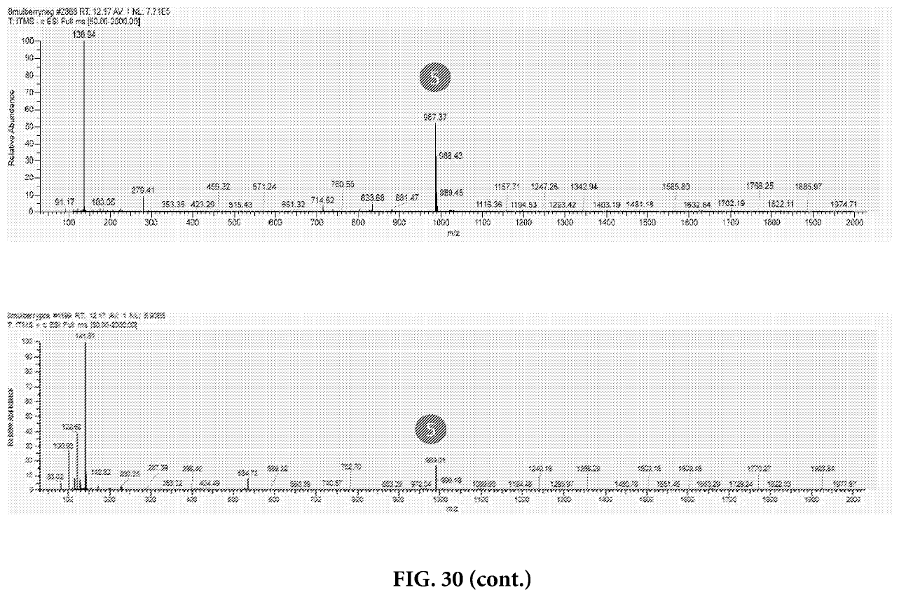

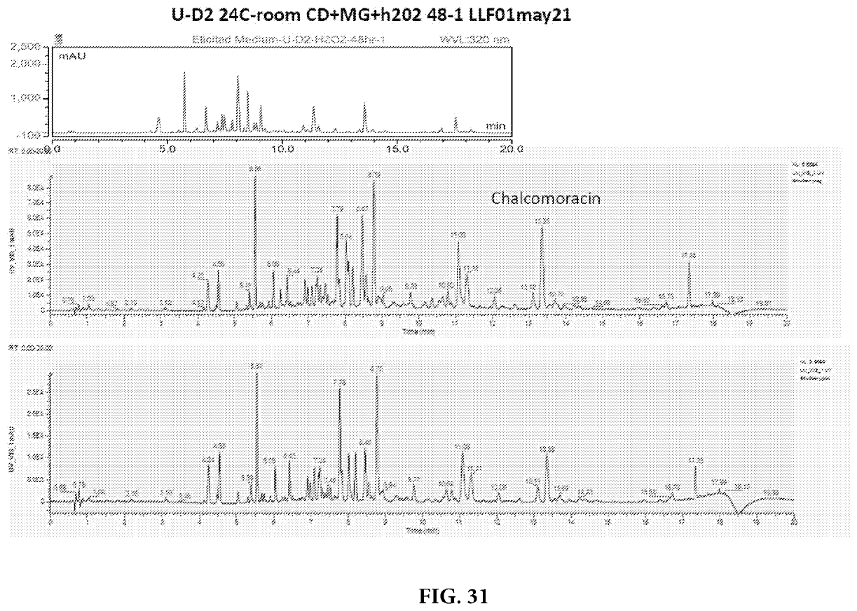

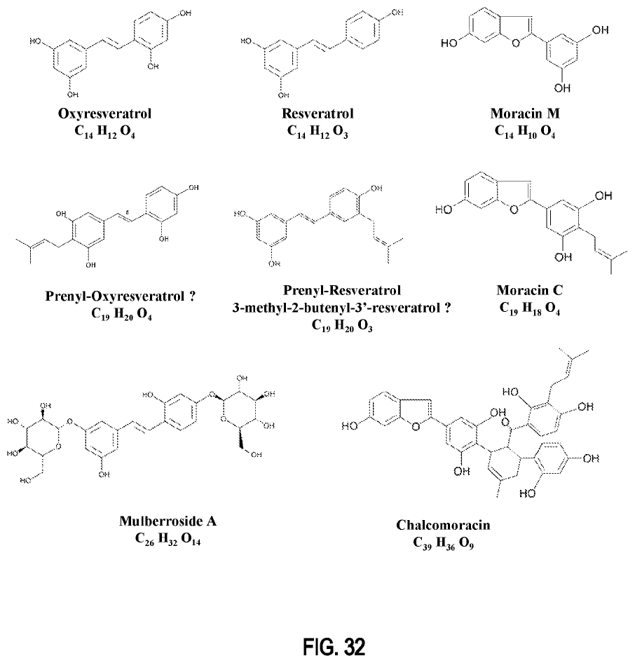

18. The method of claim 15, wherein the botanical composition is characterized by a spectrum that is substantially the same as the spectrum shown in FIGS. 19, 21, 26, 28, 30, and/or 43.

19. The method of claim 15, wherein the botanical composition further comprises a stilbenoid, flavone, or derivative thereof.

20. The method of claim 19, wherein the stilbenoid or derivative thereof comprises resveratrol, oxyresveratrol, mulberroside, prenyl-resveratrol, prenyloxyresveratrol, or combination thereof.

21. The method of claim 20, wherein oxyresveratrol comprises about 0.1 mg/g to about 10.0 mg/g per dry weight of root tissue.

22. The method of claim 20, wherein resveratrol comprises about 0.1 mg/g to about 10.0 mg/g per dry weight of root tissue.

23. The method of claim 20, wherein prenyl-resveratrol comprises about 0.1 mg/g to about 5.0 mg/g per dry weight of root tissue.

24. The method of claim 20, wherein mulberroside A comprises about 0.1 mg/g to about 20.0 mg/g per dry weight of root tissue.

25. The method of claim 20, wherein prenyloxyresveratrol comprises about 0.1 mg/g to about 20 mg/g per dry weight of root tissue.

26. The method of claim 19, wherein the flavone or derivative thereof comprises kuwanon or a derivative thereof.

27. The method of claim 26, wherein the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E.

28. The method of claim 27, wherein kuwanon C comprises about 0.1 mg/g to about 10.0 mg/g per dry weight of root tissue.

29. The method of claim 27, wherein kuwanon H comprises about 0.1 mg/g to about 10.0 mg/g per dry weight of root tissue.

30. The method of claim 27, wherein the kuwanon G analog comprises about 0.1 mg/g to about 10.0 mg/g per dry weight of root tissue.

31. The method of claim 1, wherein the plant tissue comprises the root and/or the aerial parts of Morus alba.

32. The method of claim 1, wherein the root culture is a not a hairy root culture.

33. The method of claim 1, wherein the root culture is a hairy root culture.

34. A method of producing moracin in a botanical composition, the method comprising
obtaining plant tissue from Morus alba;
producing a root culture from the plant tissue; and
stimulating production of a botanical composition from said root culture using a combination of elicitors.

35. The method of claim 34, further comprising culturing the plant tissue for a period of time in a culture medium, such that the plant tissue produces a root culture.

36. The method of claim 35, wherein the time in the culture medium comprises less than 1 day, about 1 day, about 5 days, about 10 days, about 20 days, about 25 days, about 30 days, or more than 30 days.

37. The method of claim 34, further comprising incubating the root culture in the elicitation medium for about 4 hours, about 12 hours, about 24 hours, about 48 hours, about 96 hours, or about 192 hours.

38. The method of claim 34, wherein the root culture comprises root tissue, medium of said root culture, or a combination thereof.

39. The method of claim 38, further comprising the step of isolating the botanical composition from the root culture, thereby providing a botanical extract.

40. The method of claim 39, comprising contacting the root culture with a non-polar solvent and extracting the organic layer from the aqueous layer.

41. The method of claim 40, wherein the non-polar solvent comprises ethyl acetate.

42. The method of claim 39, comprising contacting the root culture with a polar solvent and extracting the organic layer from the aqueous layer.

43. The method of claim 42, wherein the polar solvent comprises water, an alcohol, a glycol, or a combination thereof.

44. The method of claim 43, wherein the alcohol comprises methanol, ethanol, propanol, butanol, pentanol, and hexanol.

45. The method of claim 43, wherein the glycol comprises ethylene glycol or propylene glycol.

46. The method of either claim 34 or claim 37, wherein the elicitors are selected from the group consisting of cyclodextrin, magnesium chloride, hydrogen peroxide, or a combination thereof.

47. The method of any one of claim 34, 37, or 46, wherein the elicitors can be present in a concentration comprising about 18 g/L cyclodextrin, about 1 mM magnesium chloride, about 3 mM hydrogen peroxide, or any combination thereof.

48. The method of claim 39, wherein the botanical composition comprises about 0.01 mg/g to about 50 mg/g of an aryl benzofuran compound or derivative thereof per dry weight of root tissue.

49. The method of claim 48, wherein the aryl benzofuran compound is a compound comprising Formula (I): 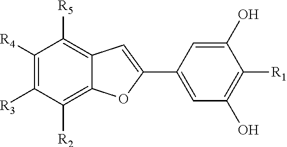
wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof.

50. The method of claim 49, wherein the compound comprising Formula (I) comprises: 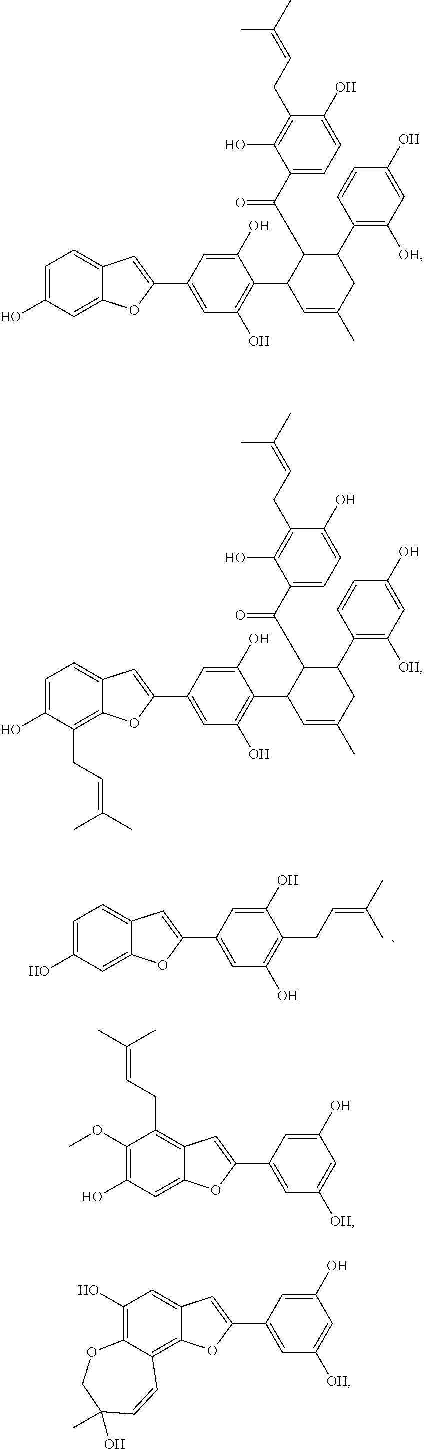 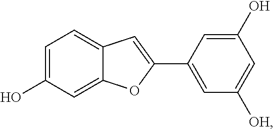 [SMILES: Oc1cc(O)cc(-c2cc3ccc(O)cc3o2)c1]
or
a glucosidyl derivative thereof.

51. The method of claim 48, wherein the botanical composition is characterized by a spectrum that is substantially the same as the spectrum shown in FIGS. 19, 21, 26, 28, 30, and/or 43.

52. The method of claim 48, wherein the botanical composition further comprises a stilbenoid, flavone, or derivative thereof.

53. The method of claim 52, wherein the stilbenoid or derivative thereof comprises resveratrol, oxyresveratrol, mulberroside, prenyl-resveratrol, prenyloxyresveratrol, or combination thereof.

54. The method of claim 53, wherein oxyresveratrol comprises about 0.1 mg/g to about 10.0 mg/g per dry weight of root tissue.

55. The method of claim 53, wherein resveratrol comprises about 0.1 mg/g to about 10.0 mg/g per dry weight of root tissue.

56. The method of claim 53, wherein prenyl-resveratrol comprises about 0.1 mg/g to about 5.0 mg/g per dry weight of root tissue.

57. The method of claim 53, wherein mulberroside A comprises about 0.1 mg/g to about 20.0 mg/g per dry weight of root tissue.

58. The method of claim 53, wherein prenyloxyresveratrol comprises about 0.1 mg/g to about 20 mg/g per dry weight of root tissue.

59. The method of claim 52, wherein the flavone or derivative thereof comprises kuwanon or a derivative thereof.

60. The method of claim 59, wherein the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E.

61. The method of claim 60, wherein kuwanon C comprises about 0.1 mg/g to about 10.0 mg/g per dry weight of root tissue.

62. The method of claim 60, wherein kuwanon H comprises about 0.1 mg/g to about 10.0 mg/g per dry weight of root tissue.

63. The method of claim 60, wherein the kuwanon G analog comprises about 0.1 mg/g to about 10.0 mg/g per dry weight of root tissue.

64. The method of claim 34, wherein the plant tissue comprises the root and/or the aerial parts of Morus alba.

65. The method of claim 34, wherein the root culture is a not a hairy root culture.

66. The method of claim 34, wherein the root culture is a hairy root culture.

67. A botanical composition produced by the method of claim 1 or claim 34.

68. The botanical composition of claim 67, wherein the botanical composition comprises about 0.01 mg/g to about 50 mg/g of an aryl benzofuran compound or derivative thereof per dry weight of root tissue.

69. The botanical composition of claim 68, wherein the aryl benzofuran compound is a compound comprising Formula (I): 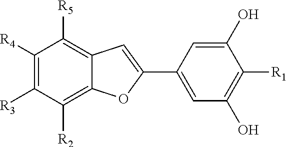
wherein, R₁, R₂, R₃, R₄, and R₅ are independently chosen from hydrogen, alkyl, aryl, alkoxy, aryloxy, alkenyl, esteryl, cycloalkyl, heterocyclyl, a polycyclic group, a prenyl group, glucosyloxy, glucosidyl, or derivative thereof.

70. The botanical composition of claim 69, wherein the compound comprising Formula (I) comprises: 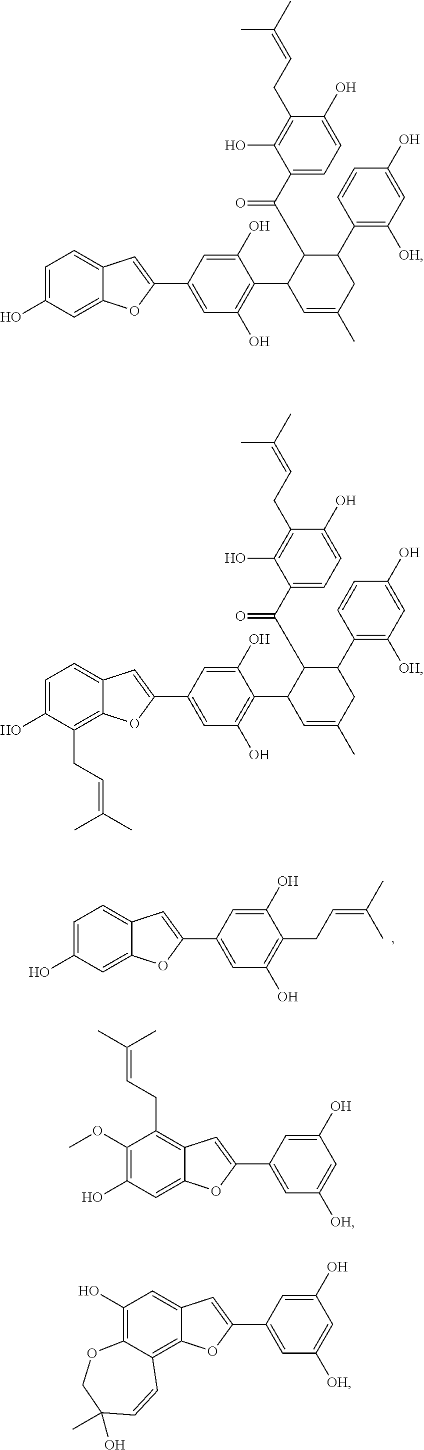 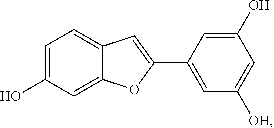 [SMILES: Oc1cc(O)cc(-c2cc3ccc(O)cc3o2)c1]
or
a glucosidyl derivative thereof.

71. The botanical composition of claim 68, wherein the botanical composition is characterized by a spectrum that is substantially the same as the spectrum shown in FIGS. 19, 21, 26, 28, 30, and/or 43.

72. The botanical composition of claim 68, wherein the botanical composition further comprises a stilbenoid, flavone, or derivative thereof.

73. The botanical composition of claim 72, wherein the stilbenoid or derivative thereof comprises resveratrol, oxyresveratrol, mulberroside, prenyl-resveratrol, prenyloxyresveratrol, or combination thereof.

74. The botanical composition of claim 73, wherein oxyresveratrol comprises about 0.1 mg/g to about 10.0 mg/g per dry weight of root tissue.

75. The botanical composition of claim 73, wherein resveratrol comprises about 0.1 mg/g to about 10.0 mg/g per dry weight of root tissue.

76. The botanical composition of claim 73, wherein prenyl-resveratrol comprises about 0.1 mg/g to about 5.0 mg/g per dry weight of root tissue.

77. The botanical composition of claim 73, wherein mulberroside A comprises about 0.1 mg/g to about 20.0 mg/g per dry weight of root tissue.

78. The botanical composition of claim 73, wherein prenyloxyresveratrol comprises about 0.1 mg/g to about 20 mg/g per dry weight of root tissue.

79. The botanical composition of claim 72, wherein the flavone or derivative thereof comprises kuwanon or a derivative thereof.

80. The botanical composition of claim 79, wherein the kuwanon compound or derivative thereof comprises kuwanon V, kuwanon I, kuwanon Q, kuwanol A, sanggenon F, kuwanol E, kuwanon C, kuwanon G (Moracenin B), kuwanon H (moracenin A), kuwanon-G analog, 2′,4′,7′-trihydroxyflavanone, australisin B, or wittiorumin E.

81. The botanical composition of claim 80, wherein kuwanon C comprises about 0.1 mg/g to about 10.0 mg/g per dry weight of root tissue.

82. The botanical composition of claim 80, wherein kuwanon H comprises about 0.1 mg/g to about 10.0 mg/g per dry weight of root tissue.

83. The botanical composition of claim 80, wherein the kuwanon G analog comprises about 0.1 mg/g to about 10.0 mg/g per dry weight of root tissue.

84. The botanical composition of claim 32, further comprising a carrier, excipient or dilutant.

85. A fraction or an isolated component of the botanical composition produced by the method of claim 1 or claim 30.

86. The fraction or isolated component of claim 29, further comprising a carrier, excipient, or diluent.
